(19) United States
(12) Patent Application Publication
(10) Pub. No.: US 2011/0136896 A1
(43) Pub. Date: Jun. 9, 2011

(54) VARIANT HCMV PP65, IE1, AND IE2 POLYNUCLEOTIDES AND USES THEREOF

(76) Inventors: Tong-Ming Fu, Maple Glen, PA (US); Danilo R. Casimiro, Harleysville, PA (US); Daniel C. Freed, Limerick, PA (US); Aimin Tang, Landsdale, PA (US)

(21) Appl. No.: 13/056,899

(22) PCT Filed: Jul. 28, 2009
(86) PCT No.: PCT/US09/51895

§ 371 Date: Jan. 31, 2011

Related U.S. Application Data
(60) Provisional application No. 61/137,685, filed on Aug. 1, 2008.

(51) Int. Cl. A61K 31/7088; C07H 21/04; C07K 14/005; C12N 15/63; C12P 21/06

(52) U.S. Cl. 514/44 R; 536/23.72; 530/350; 435/320.1; 435/69.1

(57) ABSTRACT

The present invention relates to compositions and methods to elicit or enhance cell-mediated immunity against HCMV infection by providing polynucleotides encoding variant HCMV pp65, IE1, and IE2 proteins, and fusion proteins thereof. The present invention also provides recombinant vectors including, but not limited to, adenovirus and plasmid vectors comprising said polynucleotides and host cells comprising said recombinant vectors. Also provided herein are purified forms of the variant HCMV pp65, IE1, and IE2 proteins described herein, and fusion proteins. The variant HCMV proteins, and fusion proteins thereof, are useful as vaccines for the protection from and/or treatment of HCMV infection. Said vaccines are useful as a monotherapy or a part of a therapeutic regime, said regime comprising administration of a second vaccine such as a polynucleotide, cell-based, protein or peptide-based vaccine.

### FIELD OF THE INVENTION

The present invention relates generally to pharmaceutical products (e.g., vaccines) for eliciting cellular immune responses against human cytomegalovirus (HCMV). More specifically, the present invention relates to polynucleotide compositions which, when directly introduced into mammalian tissue, express modified forms of the HCMV proteins, pp65, IE1 and/or IE2. The present invention also provides recombinant vectors and host cells comprising said polynucleotides, purified proteins, and methods for eliciting or enhancing a cellular immune response against cytomegalovirus infections using the compositions and molecules disclosed herein.

### BACKGROUND OF THE INVENTION

Human cytomegalovirus (HCMV) is a prototype β-herpes virus, with hallmarks of persistent infection in a host (Mocarski, Edward S. “Cytomegaloviruses and Their Replication.” Fields Virology, 3rd Edition. Ed. Bernard N. Fields. Lippincott Williams & Wilkins, 1996. 2447-2492). HCMV is a well-known pathogen in immune-suppressed patients, especially in organ and bone marrow transplantation patients. Infection or reactivation of HCMV in these patients causes serious HCMV diseases, associated with high morbidity and high incidence of graft rejection (Rozaonable and Paya, 2003, Herpes 10:60-65; Fishman, 2007, N. Engl. J. Med. 357:2601-2615). The congenital infection of HCMV can cause neurological damage in the fetus, manifested in infants as progressive neurological defects, including sensory hearing loss, mental retardation and cerebral palsy (reviewed in Dollard et al, 2007, Rev. Med. Virol. 17:355-363). It is estimated that 4000-8000 infants have health problems each year as a result of congenital HCMV infection in United States. Because of the high economic burden associated with long term care of infants suffering from neurological damages, an effective HCMV vaccine for prevention of congenital HCMV infection was assigned the highest priority by the Institute of Medicine in its report on assessment of targets for vaccine development (Committee to Study Priorities for Vaccine Development, Division of Health Promotion and Disease Prevention, & Institute of Medicine (1999). Vaccines for the 21^{st} Century: A Tool for Decision making. Washington D.C.: National Academy Press).

Both arms of adaptive immune responses, i.e., cellular immune response (e.g., helper T cell and cytotoxic T cell responses) and humoral immune response (e.g., neutralizing antibodies), are important for control of HCMV infection and prevention of congenital transmission (Revello and Gerna, 2002, Clin. Microbiol. Rev. 15:680-715; Schleiss and Heineman, 2005, Expert Rev. Vaccines 4:381-406). It is recognized that host immune responses are not sufficient to clear HCMV infection but are effective both to suppress active viral replication and dissemination and to maintain control over intermittent reactivations. Extensive analysis of immune responses in organ and bone marrow transplantation patients has indicated the importance of T cells in control of HCMV infection and HCMV diseases. Recent publications also demonstrate an inverse correlation in the development of CMV T cells during primary infection and congenital transmission in pregnant women (Lilleri et al, 2007, J. Infect. Dis. 195:1062-1070). These lines of evidence, along with animal studies with murine cytomegalovirus infection, suggest that an effective HCMV vaccine should have the ability to elicit T cell responses.

HCMV is a double stranded DNA virus with a genome size greater than 235 Kb and encodes more than 200 ORFs (Murphy et al, 2003, Proc. Natl. Acad. Sci. U.S.A. 100:14976-14981). The expression of HCMV viral genes follows distinct kinetic phases, i.e., immediately early, early and late phases. The present invention relates to HCMV vaccines for eliciting T cell responses targeting antigens early in the viral life cycle.

### SUMMARY OF THE INVENTION

The present invention relates to compositions and methods to elicit or enhance cell-mediated immunity against HCMV infection by providing polynucleotides encoding variant HCMV pp65, IE2, and IE2 proteins, and fusion proteins thereof. The variant protein comprises mutations relative to a wild-type amino acid sequence reducing nuclear localization of the protein and may contain additional alterations removing other undesirable activity.

The present invention also provides recombinant vectors including, but not limited to, adenovirus and plasmid vectors comprising said polynucleotides and host cells comprising said recombinant vectors. Also provided herein are purified forms of the variant HCMV pp65, IE2, and IE2 proteins described herein, and fusion proteins. The variant HCMV proteins, and fusion proteins thereof, are useful as vaccines for the protection from and/or treatment of HCMV infection. Said vaccines are useful as a monotherapy or a part of a therapeutic regime, said regime comprising administration of a second vaccine such as a polynucleotide, cell-based, protein or peptide-based vaccine.

In one embodiment of the present invention, the sequence of nucleotides encoding the variant HCMV pp65, IE1, and/or IE2 proteins, and fusion proteins thereof, comprises codons that have been optimized for expression in a human host cell. The transcripts of this artificial codon usage differ from native viral transcripts, preferably are not subject to regulations by viral micro RNAs, or a pose a risk of recombination with native viral genomes if used in patients with latent HCMV infection. In certain embodiments of the invention, the codon usage pattern of the polynucleotide sequence resembles that of highly expressed mammalian and/or human genes and is independent of native viral sequences of HCMV.

Another aspect of this invention is expression constructs comprising nucleotides encoding the variant HCMV pp65, IE1, and/or IE2 proteins, and fusion proteins thereof, described herein. In an embodiment, the expression construct is an adenoviral or plasmid vector comprising a nucleotide sequence that encodes a variant HCMV pp65, IE1, or IE2 protein, and fusion proteins thereof, as described herein. The expression constructs can be used in immunogenic, pharmaceutical compositions and vaccines for the protection from and/or treatment of HCMV infection.

The present invention further provides methods for both protecting against HCMV infection in a patient or treating a patient with HCMV infection, by eliciting an immune response to the variant HCMV pp65, IE1, or IE2 proteins described herein, and/or fusion proteins thereof, through administration of a vaccine or pharmaceutical composition comprising the vectors described herein.

As used throughout the specification and appended claims, the following definitions and abbreviations apply:

The term “promoter” refers to a recognition site on a DNA strand to which an RNA polymerase binds. The promoter forms an initiation complex with RNA polymerase to initiate and drive transcriptional activity. The complex can be modified by activating sequences termed “enhancers” or inhibiting sequences termed “silencers.”

The term “cassette” refers to a nucleotide or gene sequence that is to be expressed from a vector. In general, a cassette comprises a gene coding sequence that can be inserted into a vector, which in some embodiments, provides regulatory sequences for expressing the nucleotide or gene sequence. In other embodiments, the nucleotide or gene sequence provides the regulatory sequences for its expression. In further embodiments, the vector provides some regulatory sequences and the nucleotide or gene sequence provides other regulatory sequences. For example, the vector can provide a promoter for transcribing the nucleotide or gene sequence and the nucleotide or gene sequence provides a transcription termination sequence. The regulatory sequences that can be provided by the vector include, but are not limited to, enhancers, transcription termination sequences, splice acceptor and donor sequences, introns, ribosome binding sequences, and poly(A) addition sequences.

The term “vector” refers to some means by which a DNA sequence can be introduced into a host organism or host tissue. Various types of vectors include, but are not limited to, plasmid, virus (including adenovirus), bacteriophages and cosmids.

The term “first generation,” as used in reference to adenoviral vectors, describes adenoviral vectors that are replication-defective. First generation adenovirus vectors typically have a deleted or inactivated E1 gene region, and preferably have a deleted or inactivated E3 gene region.

The term “protein” or “polypeptide,” used interchangeably herein, indicates a contiguous amino acid sequence and does not provide a minimum or maximum size limitation. One or more amino acids present in the protein may contain a post-translational modification, such as glycosylation or disulfide bond formation.

As used herein, a “fusion protein” refers to a protein having at least two heterologous polypeptides covalently linked in which one polypeptide is derived from one protein sequence and the other polypeptide is derived from a second protein sequence. The fusion proteins of the present invention comprise a first polypeptide sequence of a variant HCMV protein described herein fused to a second polypeptide sequence of a second variant HCMV protein described herein. It is understood that HCMV polypeptides included within said fusion proteins include fragments, homologs, and functional equivalents of the variant HCMV proteins described herein, such as those in which one or more amino acids is inserted, deleted or replaced by other amino acid(s).

The term “treatment” refers to both therapeutic treatment and prophylactic or preventative measures. Those in need of treatment include those already with a disorder as well as those prone to have a disorder or those in which a disorder is to be prevented.

A “disorder” is any condition resulting in whole or in part from cytomegalovirus infection. Encompassed by the term “disorder” are chronic and acute disorders or diseases including those pathological conditions which predispose the mammal to the disorder in question.

The term “protect” or “protection,” when used in the context of a treatment method of the present invention, means reducing the likelihood of cytomegalovirus infection or of obtaining a disorder(s) resulting from cytomegalovirus infection, as well as reducing the severity of the infection and/or a disorder(s) resulting from such infection.

The term “effective amount” means sufficient vaccine composition that, when introduced to a mammalian host, produces an adequate level of the intended polypeptide, resulting in a protective immune response. One skilled in the art recognizes that this level may vary.

“mpp65” refers to a protein variant of wild-type HCMV pp65 disclosed in SEQ ID NO:3.

“mIE1” refers to a protein variant of wild-type HCMV IE1 disclosed in SEQ ID NO:9.

“IE2(H2A)” refers to a protein variant of wild-type HCMV IE2 disclosed in SEQ ID NO:14.

“mIE2” refers to a protein variant of wild-type HCMV IE2 disclosed in SEQ ID NO:16.

“mIE2(H2A)” refers to a protein variant of wild-type HCMV IE2 disclosed in SEQ ID NO:18.

“P12,” P21,” 2P1” and “21P” refer to fusion proteins comprising mpp65, mIE1 and mIE2 and disclosed in SEQ ID NOs: 20, 22, 24 and 26, respectively.

“Substantially similar” means that a given nucleic acid or amino acid sequence shares at least 75% sequence identity to a reference sequence. In different embodiments sequence identity is at least 85%, at least 90%, at least 95%, or at least 99%; for nucleotides, differ by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides; and/or for amino acids differ by 0, 1, 2, 3, 4, 5, 6, 7, 8, 9, 10, 11, 12, 13, 14, 15, 16, 17, 18, 19, or 20 amino acids alterations. Sequence identity to a reference sequence is determined by aligning a sequence with the reference sequence and determining the number of identical nucleotides or amino acids in the corresponding regions. This number is divided by the total number of amino acids or nucleotides in the reference sequence, multiplied by 100, and then rounded to the nearest whole number. Sequence identity can be determined by a number of art-recognized sequence comparison algorithms or by visual inspection (see generally Ausubel, F M, et al., Current Protocols in Molecular Biology, 4, John Wiley & Sons, Inc., Brooklyn, N.Y., A.1E.1-A.1F.11, 1996-2004).

A “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A “recombinant gene,” by virtue of its sequence and/or form, does not occur in nature. Examples of recombinant nucleic acid include purified nucleic acid, two or more nucleic acid regions combined together providing a different nucleic acid than found in nature, and the absence of one or more nucleic acid regions (e.g., upstream or downstream regions) that are naturally associated with each other.

The term “nucleic acid” or “nucleic acid molecule” refers to ribonucleic acid (RNA) or deoxyribonucleic acid (DNA) and can exist in various sizes (e.g., probes, oligonucleotides, fragments or portions thereof, and primers).

A “wild-type” or “wt,” in reference to a protein or gene sequence, refers to a protein or gene sequence comprising a naturally occurring sequence of amino acids. The amino acid and nucleotide sequences of wild-type HCMV pp65 are set forth in SEQ ID NO:1 and SEQ ID NO:2, respectively. The amino acid and nucleotide sequences of wild-type HCMV IE1 are set forth in SEQ ID NO:6 and SEQ ID NO:7, respectively. The amino acid and nucleotide sequences of wild-type HCMV IE2 are set forth in SEQ ID NO:11 and SEQ ID NO:12, respectively.

Reference to “isolated” indicates a different form than found in nature. The different form can be, for example, a different purity than found in nature and/or a structure that is not found in nature. An isolated protein, for example, is preferably substantially free of serum proteins. A protein substantially free of serum proteins is present in an environment lacking most or all serum proteins.

Reference to open-ended terms such as “comprises” allows for additional elements or steps. Occasionally, phrases such as “one or more” are used with or without open-ended terms to highlight the possibility of additional elements or steps.

Unless explicitly stated, reference to terms such as “a,” “an,” and “the” is not limited to one and include the plural reference unless the context clearly dictates otherwise. For example, “a cell” does not exclude “cells.” Occasionally, phrases such as one or more are used to highlight the possible presence of a plurality.

The term “mammalian” refers to any mammal, including a human being.

The abbreviation “Kb” refers to kilobases.

The abbreviation “ORF” refers to the open reading frame of a gene.

The abbreviation “Ad6” refers to adenovirus serotype 6. The abbreviation “Ad5” refers to adenovirus serotype 5.

The abbreviation “CMV” refers to cytomegalovirus. The abbreviation “HCMV” refers to human cytomegalovirus.

### BRIEF DESCRIPTION OF THE DRAWINGS

FIG. 1 shows a Western immunoblot of the expression of pp65 and mpp65 from adenoviral vectors. Lane 1, lysate from PerC.6 cells mock transfected; lane 2, lysate from PerC.6 cells transfected with Ad6-pp65; lane 3, lysate from PerC.6 cells transfected with Ad6-mpp65, and lane 4, lysate from PerC.6 cells transfected with Ad5-pp65.

FIG. 2 shows a Western immunoblot of the expression of IE1- and IE2-related proteins from plasmid DNA vectors. The individual lanes are marked.

FIG. 3 shows a Western immunoblot of the expression of IE1- and IE2-related proteins from adenoviral 6 (Ad6) vectors. The individual lanes are marked.

FIG. 4 shows results of flow cytometry analysis of splenocytes from mice vaccinated with either Ad6-pp65 (expressing wild-type pp65) or Ad-mpp65 (expressing a modified form of pp65 called mpp65). The splenocytes were stimulated with either DMSO control or a pp65 peptide pool of 15-mers overlapping by 11 amino acids.

FIGS. 5A and 5B shows result of ELISPOT assays of splenocytes from mice vaccinated with either Ad6-pp65 (A) or Ad-mpp65 (B). The splenocytes were stimulated with either DMSO control or a pp65 peptide pool of 15-mers overlapping by 11 amino acids.

FIG. 6 shows results of ELISA assay of sera collected at three weeks post immunization with either Ad6-pp65 (squares) or Ad-mpp65 (circles).

FIG. 7 shows result of ELISPOT assays of splenocytes from mice vaccinated with either Ad6-IE1 or Ad-mIE1. The splenocytes were stimulated with either DMSO control or a IE1 peptide pool of 15-mers overlapping by 11 amino acids.

FIG. 8 shows result of ELISPOT assays of splenocytes from mice vaccinated with either Ad6-IE2 or Ad-mIE2. The splenocytes were stimulated with either DMSO control or a IE2 peptide pool of 15-mers overlapping by 11 amino acids.

### DETAILED DESCRIPTION OF THE INVENTION

The present invention includes nucleic acid molecules (also referred to herein as “polynucleotides”) comprising a sequence encoding any one, any two, or all three variant HCMV pp65, IE1, and IE2 proteins described herein. The variant protein comprises mutations relative to a wild-type amino acid sequence reducing nuclear localization of the protein and may contain additional mutations removing other undesirable activity. The provided mutations facilitate the use of nucleic acid encoding the protein as a therapeutic agent.

The nucleic acid molecules and associated vectors can be used to elicit cell-mediated responses upon administration to a host, such as primate, and preferably a human. The vaccines of the present invention should lower transmission rate of HCMV infection to previously uninfected individuals, reduce levels of viral loads within a HCMV-infected individual, and/or reduce the likelihood of virus activation in the case of a latent infection. Overall, the present invention may include: (1) the administration and intracellular delivery of HCMV-based, polynucleotide vector vaccines, (2) the expression of variant HCMV proteins which are immunogenic in terms of eliciting a cell-mediated immune response, and (3) the inhibition or, at least, alteration of known, early viral functions shown to promote HCMV replication and/or reduce load within an infected host.

In one embodiment, the synthetic nucleic acid molecules of the present invention are codon-optimized polynucleotides that encode the HCMV pp65, IE1, or IE2 variants and fusion proteins comprising said variants. The variant HCMV proteins and fusion proteins disclosed within this specification may be nullified of undesired functions related to host cell cycles or transactivation while retaining the ability to be properly presented to the host major histocompatibility class I (MHC I) complex and, in turn, elicit a host T-cell response. Accordingly, the present invention provides polynucleotides, vectors, host cells, and encoded proteins comprising a variant HCMV sequence for use in vaccines and pharmaceutical compositions for the treatment of and/or protection from cytomegalovirus infection.

In order to generate a cell-mediated response, immunogens must be synthesized within (MHC I presentation) or introduced into (MHC II presentation) cells. For immunogens synthesized intracellularly, the protein is expressed and then processed into small peptides by the proteasome complex and translocated into the endoplasmic reticulum/Golgi complex secretory pathway for eventual association with MHC class I proteins. CD8⁺ T lymphocytes recognize antigens in association with class I MHC via the T-cell receptor (TCR). Activation of naive CD8⁺ T-cells into activated effector or memory cells generally requires both TCR engagement of the antigen as described above, as well as engagement of co-stimulatory proteins. Optimal induction of T-cell responses usually requires “help” in the form of cytokines from CD4⁺ T lymphocytes which recognize antigens associated with MHC class II molecules via TCRs.

The exemplified polynucleotides of the present invention encode variant HCMV proteins and include sequences synthetically manipulated using codons that are more optimal for human expression. Since the polynucleotide vaccines of the present invention may be administered to a patient with chronic, persistent infection of HCMV, this codon modification strategy ensures the following: (1) the expression of these polynucleotides is consistent and less likely to be influenced by any endogenous viral micro RNA transcript, reported as a mechanism to modulate viral gene expression (Grey and Nelson, 2008, J. Clin Virol, 41:186; Murphy et al, 2008, Proc. Nat'l Acad. Sci USA 105:5453); and, (2) there is a minimal chance of recombination between vaccine-introduced viral genes and latent HCMV viral genome. In one embodiment, the polynucleotides of the present invention comprise an open reading frame encoding a variant HCMV pp65, IE1, or IE2 protein, or fusion proteins thereof as described herein, wherein the codon usage has been optimized for expression in a mammal, especially a human. Codon optimization of the polynucleotides enhances both the immunogenic properties of the encoded proteins by enabling high level expression in a mammalian host cell and the safety of vaccines comprising said polynucleotides. In one embodiment, the following codon usage for mammalian optimization is used: Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), Ile (ATC), Asn (AAC), Cys (TGC), Ala (GCC), Gln (CAG), Phe (TTC), Asp (GAC) and Tyr (TAC). In another embodiment, the following codon usage for mammalian optimization is used: Met (ATG), Gly (GGC), Lys (AAG), Trp (TGG), Ser (TCT), Arg (AGG), Val (GTG), Pro (CCT), Thr (ACA), Glu (GAG); Len (CTG), His (CAT), Ile (ATT), Asn (AAT), Cys (TGT), Ala (GCT), Gln (CAG), Phe (TTT), Asp (GAT) and Tyr (TAT). For an additional discussion relating to mammalian (human) codon optimization, see U.S. Pat. No. 6,534,312, which is hereby incorporated by reference. Accordingly, the optimized polynucleotides may be used for the development of recombinant DNA vaccines, which provide effective protection against HCMV infection through cell-mediated immunity.

Viral protein pp65, also called UL83 protein, is a major tegument protein of 561 amino acids. The wild-type HCMV pp65 gene sequence is set forth in SEQ ID NO:2 and has been reported previously (see, e.g., NCBI Accession no. NC_{—}001347 (nucleotides 120283-121968), encoding the wild-type pp65 protein as set forth in SEQ ID NO:1 (see, NCBI Accession no P06725). The wild-type protein contains a putative kinase domain of ATP binding motifs with a highly conserved lysine residue at amino acid position 436. Wild-type pp65 also contains a bipartite nuclear localization signal (NLS). A modified HCMV pp65 protein disclosed herein as mpp65 is engineered to inactivate pp65 function by deleting or modifying portions of the bipartite NLS and substituting the conserved lysine residue at position 436 with an uncharged glycine residue. The modified protein, mpp65, expresses as a 535 amino acid protein (SEQ ID NO:3; see Example 3, infra) and is shown to be immunogenic in mice (see Example 4, infra). The sequence encoding pp65 is highly conserved among reported HCMV isolates, and modifications outlined here should apply to pp65 homologs that may exist among different strains of HCMV.

In one embodiment, the sequence of nucleotides is codon-optimized for expression in a mammalian system such as human. In a further embodiment, the wild-type pp65 amino acid sequence that is mutated is set forth in SEQ ID NO:1. Mutations may encompass amino acid additions, deletions (e.g., truncations, internal deletions) or substitutions. In one embodiment, a variant HCMV pp65 protein encoded by a polynucleotide of the present invention comprises mutations that eliminate or substantially reduce the activity of nuclear localization of wild-type pp65 by modifying known bipartite NLS (e.g., located within approximately amino acids 415-438 and 536-561 of SEQ ID NO:1, respectively). Thus, in this embodiment, a variant HCMV pp65 protein which contains mutations that eliminate or substantially reduce bipartite NLS activity can have additional amino acid mutations. For example, said variant can contain additional mutation(s) that eliminate or substantially reduce the protein kinase activity mediated by a conserved lysine residue at amino wild-type pp65 (e.g., located at amino acid position 436 of SEQ ID NO:1). Thus, in a further embodiment, a variant HCMV pp65 protein comprises the following mutations: R415G, K416G and R419G to eliminate NLS1 activity; K436G to eliminate/substantially reduce protein kinase activity; and a deletion of approximately amino acids 536-561 to eliminate/substantially reduce NLS2 activity.

Polynucleotides comprising a nucleotide sequence encoding a variant HCMV pp65 protein referred to herein as mpp65 and having an amino acid sequence as set forth in SEQ ID NO:3 (see Example 2, infra, for details) are included as part of the present invention. The present invention also includes polynucleotides comprising a nucleotide sequence encoding a variant human CMV pp65 protein that is substantially similar to SEQ ID NO:3. In one embodiment, said nucleotide sequence is codon-optimized for expression in a mammalian system such as human. A nucleotide sequence encoding the variant pp65 protein sequence as set forth in SEQ ID NO:3 is disclosed in SEQ ID NO:5. The nucleotide sequence disclosed in SEQ ID NO:5 represents a codon-optimized nucleic acid sequence that encodes mpp65. In another embodiment, the present invention includes polynucleotides that are substantially similar to SEQ ID NO:5. The modified pp65 protein exemplified herein, mpp65, is a derivative of HCMV pp65 wherein both the bipartite nuclear localization signal and putative kinase domain of the protein have been rendered substantially non-functional.

Viral proteins IE1 (491 amino acids), also called UL123, and IE2 (579 amino acids), also called UL122, are nuclear proteins important for HCMV viral gene regulation. IE1 augments major immediate early promoter (MIEP) activity, and IE2 down-regulates MIEP activity. Both proteins have been shown to modulate host cell cycles, possibly through their interactions with Rb family proteins. Expression of both IE1 and IE2 is driven by the MIEP promoter through alternative splicing. Exemplified variants of wild-type IE1 and IE2 disclosed herein are generated by the following mutations: 1) modification or removal of the well-defined, bipartite nuclear localization signals (NLSs) to reduce interaction with host proteins important for cell cycle regulation and cellular transcriptional activation factors; and, 2) removal of exon 3 to eliminate probability of activating latent HCMV. The wild-type HCMV IE1 gene sequence is set forth in SEQ ID NO:7 and has been reported previously. (See, e.g., NCBI Accession no. NC_{—}001347.2 (joining nucleotides 171937-173156, 173327-173511, and 173626-173696), encoding the wild-type IE1 protein as set forth in SEQ ID NO:6 (see, NCBI Accession no NP_{—}040060).) The wild-type HCMV IE2 gene sequence is set forth in SEQ ID NO:12 and has been reported previously (see, e.g., NCBI Accession no. NC_{—}001347.2 (joining nucleotides 170295-171781, 173327-173511, and 173626-173696), encoding the wild-type IE2 protein as set forth in SEQ ID NO:11 (see, NCBI Accession no P19893). The protein sequences for IE1 and IE2 are highly conserved among studied human CMV isolates, and modifications outlined here apply to IE1 and IE2 homologs that may exist among different strains of HCMV.

Accordingly, the present invention relates to nucleic acid molecules comprising a sequence of nucleotides that encodes a variant HCMV IE1 protein, wherein said variant comprises mutations relative to a wild-type IE1 amino acid sequence that eliminates or substantially reduces NLS activity and, optionally, exon 3 activity. The variant encoded by said polynucleotide is capable of producing an immune response in a mammal, especially a human.

In one embodiment, the sequence of nucleotides is codon-optimized for expression in a mammalian system such as human. In a further embodiment, the wild-type IE1 amino acid sequence that is mutated is set forth in SEQ ID NO:6. Mutations may encompass amino acid additions, deletions (e.g., truncations, internal deletions) or substitutions. In one embodiment, a variant HCMV IE1 protein encoded by a polynucleotide of the present invention comprises mutations that eliminate or substantially reduce the activity of NLS1 and NLS2 of wild-type IE1 (e.g., located between approximately amino acids, 2-25 and 326-342 of SEQ ID NO:6, respectively). Thus, in this embodiment, a variant HCMV IE1 protein which contains mutations that eliminate or substantially reduce bipartite NLS activity can have additional amino acid mutations. For example, said variant can contain additional mutations that eliminate or substantially reduce exon 3 activity (e.g., located between approximately amino acids 25-85 of SEQ ID NO:6). Thus, in one embodiment, a variant HCMV IE1 protein comprises the following mutations: a deletion of approximately amino acids 2-76 to eliminate/substantially reduce NLS1 activity and to remove a majority of IE1 encoded by exon 3 to eliminate/substantially reduce exon 3 activity; and, K340G, R341G and R342G to eliminate/substantially reduce NLS2 activity.

The present invention further relates to polynucleotides comprising a nucleotide sequence encoding a variant HCMV IE1 protein referred to herein as mIE1 and having an amino acid sequence as set forth in SEQ ID NO:9 (see Example 2, infra, for details). The present invention also includes polynucleotides comprising a nucleotide sequence encoding a variant HCMV IE1 protein that is substantially similar to SEQ ID NO:9. In one embodiment, said nucleotide sequence is codon-optimized for expression in a mammalian system such as human. A nucleotide sequence encoding the variant IE1 sequence as set forth in SEQ ID NO:9 is disclosed in SEQ ID NO:10. The nucleotide sequence disclosed in SEQ ID NO:10 represents a codon-optimized nucleic acid sequence that encodes mIE1. In another embodiment, the present invention includes polynucleotides that are substantially similar to SEQ ID NO:10. The modified IE1 protein exemplified herein, mIE1, is a derivative of wild-type HCMV IE1 wherein the bipartite nuclear localization signal has been rendered substantially non-functional and exon 3 has been removed to eliminate the probability of activating latent HCMV.

The present invention further relates to nucleic acid molecules comprising a nucleotide sequence encoding a variant HCMV IE2 protein. In one embodiment, said nucleotide sequence is codon-optimized for expression in a mammalian system such as human. In a further embodiment, the present invention relates to nucleic acid molecules comprising a sequence of nucleotides that encodes a variant HCMV IE2 protein, wherein said variant comprises mutations relative to a wild-type IE2 amino acid sequence that eliminate or substantially reduce NLS activity. Thus, in this embodiment, a variant HCMV IE2 protein which contains mutations that eliminate or substantially reduce bipartite NLS activity can have additional amino acid mutations. For example, said variant can contain additional mutations that eliminate or substantially reduce exon 3 activity and/or mutations that nullify the ability of the variant IE2 protein to negatively regulate WIMP activity. In another embodiment, a variant HCMV IE2 protein comprises mutations that nullify the ability of the protein to negatively regulate MIEP activity. In a further embodiment, the wild-type IE2 amino acid sequence that is mutated is set forth in SEQ ID NO:11. Mutations may encompass amino acid additions, deletions (e.g., truncations, internal deletions) or substitutions.

In one embodiment, a variant HCMV IE2 protein encoded by a polynucleotide of the present invention comprises mutations that both eliminate or substantially reduce the activity of NLS1 and NLS2 of wild-type IE2 (e.g., located between approximately amino acids 145-154 and 322-329 of SEQ ID NO:11) and exon 3 activity (e.g., located between approximately amino acids 25-85 of SEQ ID NO:11). Thus, in a further embodiment, a variant HCMV IE2 protein comprises the following mutations: R146S, K147S and K148G to eliminate/substantially reduce NLS1 activity; K324S, K325S and K326G to eliminate/substantially reduce NLS2 activity; and, a deletion of approximately amino acids 2-85 to remove exon 3 of IE2. In a still further embodiment, this variant HCMV IE2 protein further comprises H447A and H453A mutations to nullify the ability of variant IE2 to negatively regulate MIEP activity. In a still further embodiment, a variant HCMV IE2 protein comprises H447A and H453A mutations to nullify the ability of variant IE2 to negatively regulate MIEP activity.

Accordingly, the present invention relates to polynucleotides comprising a nucleotide sequence encoding a variant HCMV IE2 protein referred to herein as mIE2 having an amino acid sequence as set forth in SEQ ID NO:16 (see Example 2, infra, for details). The present invention also includes polynucleotides comprising a nucleotide sequence encoding a variant HCMV IE2 protein that is substantially similar to SEQ ID NO:16. A nucleotide sequence encoding the modified IE2 sequence set forth in SEQ ID NO:16 is disclosed in SEQ ID NO:17. The nucleotide sequence disclosed in SEQ ID NO:17 represents a codon-optimized nucleic acid sequence that encodes mIE2. In another embodiment, the present invention includes polynucleotides that are substantially similar to SEQ ID NO:17. The modified IE2 protein referred to herein as mIE2 is a derivative of wild-type HCMV IE2 wherein the removal of bipartite nuclear localization signal has rendered it substantially non-functional and exon 3 has been removed to eliminate the probability of activating latent HCMV.

In a further embodiment, the present invention relates to polynucleotides comprising a nucleotide sequence encoding a variant HCMV protein referred to herein as IE2(H2A) having an amino acid sequence as set forth in SEQ ID NO:14 (see Example 2, infra, for details). The present invention also includes polynucleotides comprising a nucleotide sequence encoding a variant HCMV IE2 protein that is substantially similar to SEQ ID NO:14. A nucleotide sequence encoding the modified IE2 sequence set forth in SEQ ID NO:14 is disclosed in SEQ ID NO:15. The nucleotide sequence disclosed in SEQ ID NO:15 represents a codon-optimized nucleic acid sequence that encodes IE2(H2A). In another embodiment, the present invention includes polynucleotides that are substantially similar to SEQ ID NO:15. IE2(H2A) has two amino acid mutations in comparison to the wild-type IE2 protein located at residue positions 446 and 452, each converting a histidine to an alanine. This has previously been shown to nullify the ability of IE2 to negatively regulate MIEP activity and abrogate viral replication.

In a still further embodiment, the present invention relates to polynucleotides comprising a nucleotide sequence encoding a variant HCMV IE2 protein referred to herein as mIE2(H2A) having an amino acid sequence as set forth in SEQ ID NO:18 (see Example 2, infra, for details). The present invention also includes polynucleotides comprising a nucleotide sequence encoding a variant HCMV IE2 protein that is substantially similar to SEQ ID NO:18. A nucleotide sequence encoding the modified IE2 sequence set forth in SEQ ID NO:18 is disclosed in SEQ ID NO:19. The nucleotide sequence disclosed in SEQ ID NO:19 represents a codon-optimized nucleic acid sequence that encodes mIE2(H2A). In another embodiment, the present invention includes polynucleotides that are substantially similar to SEQ ID NO:19. mIE2(H2A) has a combination of the modifications present in mIE2 and IE2(H2A).

The present invention also relates to a nucleic acid molecule comprising a sequence of nucleotides encoding a fusion protein comprising at least one of the variant HCMV proteins described herein (e.g., mpp65) fused with at least one of a different variant HCMV protein derivative described herein (e.g., mIE1). Such polynucleotides comprise a nucleotide sequence encoding one variant HCMV protein fused (directly or indirectly) in reading frame to a nucleotide sequence encoding at least a second variant HCMV protein. In one embodiment, each of the nucleotide sequences encoding said variant HCMV proteins contained within a fusion protein of the present invention is codon-optimized for expression in a mammalian system such as human.

Accordingly, in one embodiment, a nucleic acid molecule of the present invention comprises a sequence of nucleotides that encodes a fusion protein, wherein the fusion protein comprises at least one variant HCMV protein fused to a second variant HCMV protein, wherein the variant HCMV proteins are selected from the group consisting of: (i) a pp65 variant comprising mutations relative to the wild-type pp65 amino acid sequence that eliminate or substantially reduce bipartite nuclear localization signal (NLS) activity of the encoded pp65 variant; (ii) a IE1 variant comprising mutations relative to the wild-type IE1 amino acid sequence that eliminate or substantially reduce bipartite nuclear localization signal (NLS) activity of the encoded IE1 variant; and, (iii) a IE2 variant comprising mutations relative to the wild-type IE2 amino acid sequence that eliminate or substantially reduce bipartite nuclear localization signal (NLS) activity of the encoded IE2 variant; and wherein the fusion protein is capable of producing an immune response in a mammal. Thus, a variant HCMV protein comprised within a fusion protein of this embodiment and which contains mutations that eliminate or substantially reduce bipartite NLS activity and can contain additional amino acid mutations, as described herein in detail for the pp65, IE1 and IE2 variants. For example, a variant mpp65 protein contained within a fusion protein of this embodiment can contain additional mutations that eliminate or substantially reduce protein kinase activity. In a further embodiment, said fusion protein comprises all three variant HCMV proteins (i.e., a pp65 variant, a IE1 variant, and a IE2 variant). In a still further embodiment, the wild-type pp65, IE1, and IE2 amino acid sequences that are mutated are set forth in SEQ ID NO:1, SEQ ID NO:6, and SEQ ID NO:11, respectively. The nucleotide sequences encoding said variant HCMV proteins comprised within the fusion protein may be codon-optimized for expression in a mammalian system such as human. The variant HCMV pp65, IE1 and IE2 proteins that may be comprised with the fusion protein are described further herein.

In one embodiment, the present invention relates to a nucleic acid molecule comprising a sequence of nucleotides encoding a fusion protein comprising at least two of the variant HCMV proteins described herein as mpp65 (SEQ ID NO:3) or a substantially similar sequence, mIE1 (SEQ ID NO:9) or a substantially similar sequence, and mIE2 (SEQ ID NO:16) or a substantially similar sequence. In a further embodiment, the fusion protein comprises all three of said variant HCMV proteins. The order of nucleotide sequences encoding the individual, variant HCMV proteins can vary. For example, a fusion protein comprising all three of the variant HCMV proteins can be encoded by a polynucleotide which comprises three nucleotide sequences fused (directly or indirectly) together in proper reading frame in one of the following orders: mpp65-mIE1-mIE2; mpp65-mIE2-mIE1; mIE2-mpp65-mIE1; and, mIE2-mIE1-mpp65. In a further embodiment, to reduce the probability of generating undesired and/or auto-immunogenic T-cell epitopes due to the direct fusion of two open reading frames (ORFs), a DNA fusion linker encoding a small number of inert amino acids can be inserted between the encoding nucleotide sequences. In one embodiment, said fusion linker encodes a peptide comprising the following five inert amino acids: glycine-glycine-serine-glycine-glycine (GGSGG; SEQ ID NO:29).

Accordingly, the present invention relates to polynucleotides comprising a nucleotide sequence encoding a fusion protein referred to herein as P12 having an amino acid sequence as set forth in SEQ ID NO:20 (see Example 6, infra, for details). The present invention also includes polynucleotides comprising a nucleotide sequence encoding a fusion protein that is substantially similar to SEQ ID NO:20. P12 is a fusion protein comprising the amino acid sequences of mpp65, mIE1, and mIE2 fused together in the following order: mpp65-mIE1-mIE2. A GGSGG (SEQ ID NO:29) peptide links the mpp65 and mIE1 amino acid sequences, as well as the mIE1 and mIE2 amino acid sequences. In one embodiment, one, two, or all three of the nucleotide sequences encoding the variant HCMV antigens within P12 is codon-optimized for expression in a mammalian system such as human. A nucleotide sequence encoding the P12 fusion protein is disclosed in SEQ ID NO:21 (see Example 6, infra, for details). In another embodiment, the present invention includes polynucleotides that are substantially similar to SEQ ID NO:21.

The present invention further relates to polynucleotides comprising a nucleotide sequence encoding a fusion protein referred to herein as P21 having an amino acid sequence as set forth in SEQ ID NO:22 (see Example 6, infra, for details). The present invention also includes polynucleotides comprising a nucleotide sequence encoding a fusion protein that is substantially similar to SEQ ID NO:22. P21 is a fusion protein comprising the amino acid sequences of mpp65, mIE1, and mIE2 fused together in the following order: mpp65-mIE2-mIE1. A GGSGG (SEQ ID NO:29) peptide links the mpp65 and mIE2 amino acid sequences, as well as the mIE2 and mIE1 amino acid sequences. In one embodiment, one, two, or all three of the nucleotide sequences encoding the variant HCMV antigens within P21 is codon-optimized for expression in a mammalian system such as human. A nucleotide sequence encoding the P21 fusion protein is disclosed in SEQ ID NO:23 (see Example 6, infra, for details). In another embodiment, the present invention includes polynucleotides that are substantially similar to SEQ ID NO:23.

The present invention further relates to polynucleotides comprising a nucleotide sequence encoding a fusion protein referred to herein as 2P1 having an amino acid sequence as set forth in SEQ ID NO:24 (see Example 6, infra, for details). The present invention also includes polynucleotides comprising a nucleotide sequence encoding a fusion protein that is substantially similar to SEQ ID NO:24. 2P1 is a fusion protein comprising the amino acid sequences of mpp65, mIE1, and mIE2 fused together in the following order: mIE2-mpp65-mIE1. A GGSGG (SEQ ID NO:29) peptide links the mIE2 and mpp65 amino acid sequences, as well as the pp65 and mIE1 amino acid sequences. In one embodiment, one, two, or all three of the nucleotide sequences encoding the variant HCMV antigens within 2P1 is codon-optimized for expression in a mammalian system such as human. A nucleotide sequence encoding the 2P1 fusion protein is disclosed in SEQ ID NO:25 (see Example 6, infra, for details). In another embodiment, the present invention includes polynucleotides that are substantially similar to SEQ ID NO:25.

The present invention further relates to polynucleotides comprising a nucleotide sequence encoding a fusion protein referred to herein as 21P having an amino acid sequence as set forth in SEQ ID NO:26 (see Example 6, infra, for details). The present invention also includes polynucleotides comprising a nucleotide sequence encoding a fusion protein that is substantially similar to SEQ ID NO:26. 21P is a fusion protein comprising the amino acid sequences of mpp65, mIE1, and mIE2 fused together in the following order: mIE2-mIE1-mpp65. A GGSGG (SEQ ID NO:29) peptide links the mIE2 and mIE1 amino acid sequences, as well as the mIE1 and mpp65 amino acid sequences. In one embodiment, one, two, or all three of the nucleotide sequences encoding the variant HCMV antigens within 21P is codon-optimized for expression in a mammalian system such as human. A nucleotide sequence encoding the 21P fusion protein is disclosed in SEQ ID NO:27. In another embodiment, the present invention includes polynucleotides that are substantially similar to SEQ ID NO:27.

Exemplary polynucleotides of the present invention comprise a sequence of nucleotides as set forth in SEQ ID NOs: 5, 10, 15, 17, 19, 21, 23, 25, and 27, which encode exemplary variant HCMV pp65, IE1, or IE2 proteins, and fusion proteins thereof, of the present invention. Each of the exemplified polynucleotides comprise codons optimized for expression in a mammalian host, especially a human host.

A “triplet” codon of four possible nucleotide bases can exist in over 60 variant forms. Because these codons provide the message for only 20 different amino acids (as well as transcription initiation and termination), some amino acids can be coded for by more than one codon, a phenomenon known as codon redundancy. Thus, due to this degeneracy of the genetic code, a large number of different encoding nucleic acid sequences can be used to code for a particular protein. Amino acids are encoded by the following RNA codons:

A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

For reasons not completely understood, alternative codons are not uniformly present in the endogenous DNA of differing types of cells. Indeed, there appears to exist a variable natural hierarchy or “preference” for certain codons in certain types of cells. The implications of codon preference phenomena on recombinant DNA techniques are evident, and the phenomenon may serve to explain many prior failures to achieve high expression levels of exogenous genes in successfully transformed host organisms. This phenomenon suggests that synthetic genes which have been designed to include a projected host cell's preferred codons provide an optimal form of foreign genetic material for practice of recombinant DNA techniques.

Thus, one aspect of this invention is polynucleotides encoding variant HCMV proteins that are codon-optimized for expression in a human cell. The use of alternative codons encoding the same protein sequence may remove the constraints on expression of exogenous protein in human cells. Additionally, using codons that are more optimal for human expression reduces both the possibility of endogenous viral micro RNA transcripts from influencing expression and the possibility of the vaccine-induced gene from recombining with latent HCMV viral genome.

In accordance with some embodiments of the present invention, the nucleic acid molecules which encode the variant HCMV proteins disclosed throughout this specification are converted to polynucleotide sequences having an identical translated sequence but with alternative codon usage as described by Lathe, “Synthetic Oligonucleotide Probes Deduced from Amino Acid Sequence Data: Theoretical and Practical Considerations” J. Molec. Biol. 183:1-12 (1985), which is hereby incorporated by reference. The methodology generally consists of identifying codons in the wild-type sequence that are not commonly associated with highly expressed human genes and replacing them with more optimal codons for expression in human cells. The new gene sequence is then inspected for undesired sequences generated by these codon replacements (e.g., “ATTTA” sequences, inadvertent creation of intron splice recognition sites, unwanted restriction enzyme sites, etc.). Undesirable sequences are eliminated by substitution of the existing codons with different codons coding for the same amino acid.

It is understood that this procedure will not necessarily result in a polynucleotide sequence in which all of the codons are optimal codons according to the codon usage of highly expressed human and/or mammalian cells. However, in embodiments of the invention wherein codon-optimized polynucleotides of the variant HCMV proteins described herein are contemplated, a substantial portion of the resulting codons resemble the codon usage of highly expressed human and/or mammalian genes. Thus, in one embodiment, a “codon-optimized” polynucleotide disclosed herein comprises at least 50% of its codons that are preferred for expression in human and/or mammalian cells. In a further embodiment at least 60%, at least 70%, at least 80%, or at least 90% of the codons are preferred for expression in human and/or mammalian cells. In another embodiment, those codons preferred for expression in human and/or mammalian cells are as follows: Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), Ile (ATC), Asn (AAC), Cys (TGC), Ala (GCC), Gln (CAG), Phe (TTC), Asp (GAC) and Tyr (TAC).

As an example to illustrate a codon-optimization process used herein, the non codon-optimized nucleic acid sequence that encodes mpp65, mpp65 (nuc), is set forth in SEQ ID NO: 4 and consists of 535 codons. The codon-optimized version of this nucleic acid sequence, mpp65.syn, set forth in SEQ ID NO: 5, contains approximately 334 codons that are preferred for expression in human and/or mammalian cells, wherein the preferred codons are Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), Ile (ATC), Asn (AAC), Cys (TGC), Ala (GCC), Gln (CAG), Phe (TTC), Asp (GAC) and Tyr (TAC). This represents approximately 62% of the codons encoding the mpp65 polypeptide. It is important to note that not all of the preferred codons within mpp65.syn are generated as a result of mutating the mpp65 (nuc) sequence (i.e., some of the viral codons fall within the list of preferred codons recited above). Furthermore, there are instances where a non-preferred codon present within the viral gene sequence is mutated to another non-preferred codon. There are also instances when a viral codon that falls within the list of preferred codons recited above is mutated to a non-preferred codon.

The methods described above were used to create synthetic gene sequences which encode variant HCMV pp65, IE1, and IE2 proteins, resulting in a gene comprising codons optimized for expression in human cells. While the above procedure provides a summary of a representative methodology for designing codon-optimized genes for use in HCMV polynucleotide vaccines, it is understood by one skilled in the art that similar vaccine efficacy or expression levels of genes may be achieved by minor variations in the procedure or by minor variations in the nucleotide sequence. Thus, one of skill in the art will also recognize that additional nucleic acid molecules may be constructed that provide for more optimal expression of the disclosed, variant HCMV proteins in human cells, wherein only a portion of the codons of the DNA molecules are codon-optimized.

The present invention also relates to an isolated nucleic acid molecule, regardless of codon usage, which expresses the variant HCMV proteins described herein. Thus, it is within the scope of the present invention to utilize “non-codon optimized” version of the constructs disclosed herein, especially versions which are shown to promote a substantial cellular immune response subsequent to host administration.

Polynucleotides encoding variants of the modified HCMV pp65, IE1 and IE2 proteins described herein, or fusion proteins thereof, are also included in the present invention, including but not limited to variants generated by conservative amino acid substitutions, amino-terminal truncations, carboxyl-terminal truncations, deletions, or additions. Preferred variants, fragments and/or mutants encoded by said polynucleotides at least substantially mimic the immunological properties of the variant HCMV pp65, IE1 or IE2 proteins, or fusion proteins thereof, as set forth in the amino acid sequences disclosed herein (e.g., SEQ ID NOs: 3, 9, 14, 16, 18, 20, 22, 24, 26). For example, substitution of valine for leucine, arginine for lysine, or asparagine for glutamine may not cause a change in the desired functionality of the polypeptide, such as the ability to elicit an immune response. Thus, a “conservative amino acid substitution” refers to the replacement of one amino acid residue by another, chemically similar, amino acid residue. Examples of such conservative substitutions are: substitution of one hydrophobic residue for another; and substitution of one polar residue for another polar residue of the same charge. Table 1 provides a list of groups of amino acids, wherein one member of the group is a conservative substitution for another member.

TABLE 1
Conservative Substitutions

Ala, Val, Ile, Leu, Met
Ser, Thr
Tyr, Trp
Asn, Gln
Asp, Glu
Lys, Arg, His

Accordingly, also included within the scope of this invention are polynucleotides comprising nucleotide sequences that encode further variants of the variant HCMV pp65, IE1, or IE2 proteins, or fusion proteins thereof, disclosed herein (e.g., SEQ ID NOs: 3, 9, 14, 16, 18, 20, 22, 24, and 26) able to induce an immune response and preferably having physical properties that are substantially the same as those of the expressed protein derivatives. In one embodiment, polynucleotides encoding further variants of the variant HCMV CMV pp65, IE1, and IE2 proteins, and fusion proteins thereof, described supra comprise a nucleotide sequence that encodes an amino acid sequence that differs by 1, 2, 3, 4, 5, 6, 7, 8, 9, 10, 11, 12, 13, 14, 15, 16, 17, 18, 19, or 20 amino acid alterations from SEQ ID NOs: 3, 9, 14, 16, 18, 20, 22, 24, or 26. Each amino acid alteration is independently an addition, deletion or substitution. In another embodiment, polynucleotides encoding further variants of the variant HCMV pp65, IE1, and IE2 proteins, and fusion proteins thereof, disclosed herein comprise a nucleotide sequence that encodes an amino acid sequence that is at least 90%, at least 95% or at least 99% identical to the amino acid sequences of SEQ ID NOs: 3, 9, 14, 16, 18, 20, 22, 24, or 26. In a further embodiment, the exemplified nucleotide sequences disclosed herein (e.g., SEQ ID NOs: 5, 10, 15, 17, 19, 21, 23, 25, and 27) that encode the variant HCMV proteins and fusion proteins of the present invention are modified to encode said further variants.

The present invention also includes variants of the exemplified polynucleotides described herein (e.g., SEQ ID NOs: 5, 10, 15, 17, 19, 21, 23, 25, and 27), wherein said polynucleotide variants encode the exemplified HCMV protein variants (e.g., SEQ ID NOs: 3, 9, 14, 16, 18, 20, 22, 24, or 26). In one embodiment, said variant polynucleotides comprise a nucleotide sequence that differs by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides from SEQ ID NOs: 5, 10, 15, 17, 19, 21, 23, 25, and 27. In another embodiment, the variant polynucleotides comprise a nucleotide sequence that is at least 80%, at least 85%, at least 90%, at least 95%, or at least 99% identical to the nucleotide sequence of SEQ ID NOs: 5, 10, 15, 17, 19, 21, 23, 25, and 27.

Also included within the scope of the present invention are DNA sequences that hybridize to the complement of SEQ ID NOs: 5, 10, 15, 17, 19, 21, 23, 25, and 27 under stringent conditions. By way of example, and not limitation, a procedure using conditions of high stringency is described. Prehybridization of filters containing DNA is carried out for about 2 hours to overnight at about 65° C. in buffer composed of 6×SSC, 5×Denhardt's solution, and 100 μg/ml denatured salmon sperm DNA. Filters are hybridized for about 12 to 48 hrs at 65° C. in prehybridization mixture containing 100 μg/ml denatured salmon sperm DNA and 5-20×10⁶ cpm of ³²P-labeled probe. Washing of filters is done at 37° C. for about 1 hour in a solution containing 2×SSC, 0.1% SDS. This is followed by a wash in 0.1×SSC, 0.1% SDS at 50° C. for 45 minutes before autoradiography. Other procedures using conditions of high stringency would include either a hybridization step carried out in 5×SSC, 5×Denhardt's solution, 50% formamide at about 42° C. for about 12 to 48 hours or a washing step carried out in 0.2×SSPE, 0.2% SDS at about 65° C. for about 30 to 60 minutes. Reagents mentioned in the foregoing procedures for carrying out high stringency hybridization are well known in the art. Details of the composition of these reagents can be found in Sambrook et al., Molecular Cloning: A Laboratory Manual 2^{nd} Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001). In addition to the foregoing, other conditions of high stringency which may be used are also well known in the art.

As stated above, in some embodiments of the present invention, the synthetic molecules comprise a sequence of nucleotides, wherein some of the nucleotides have been altered so as to use the codons preferred by a human cell, thus allowing for high-level protein expression in a human host cell. Expression vectors comprising the synthetic molecules may be used as a source of a variant HCMV protein, or fusion protein thereof, which may be used in a HCMV subunit vaccine to provide effective immunoprophylaxis against HCMV infection through cell-mediated immunity.

Also provided by the present invention are purified forms of the variant HCMV proteins as described throughout this specification, and fusion proteins thereof, encoded by the nucleic acids disclosed herein. In an exemplary embodiment of this aspect of the invention, a variant HCMV pp65 protein comprises a sequence of amino acids as disclosed in SEQ ID NO:3. In another exemplary embodiment, a variant HCMV IE1 protein comprises a sequence of amino acids as disclosed in SEQ ID NO:9. In a further exemplary embodiment, a variant HCMV IE2 protein comprises a sequence of amino acids selected from the group consisting of: SEQ ID NOs: 14, 16, and 18. In another exemplary embodiment, a fusion protein comprising variant HCMV pp65, mIE1, and mIE2 proteins comprises a sequence of amino acids selected from the group consisting of: SEQ ID NOs: 20, 22, 24, and 26.

Following expression of a variant HCMV protein, or fusion protein thereof, as described herein in a recombinant host cell, said polypeptide may be recovered to provide purified protein. Several protein purification procedures are available and suitable for use. Recombinant protein may be purified from cell lysates and extracts by various combinations of, or individual application of salt fractionation, ion exchange chromatography, size exclusion chromatography, hydroxylapatite adsorption chromatography and hydrophobic interaction chromatography. In addition, recombinant protein can be separated from other cellular proteins by use of an immunoaffinity column made with monoclonal or polyclonal antibodies that cross-react with the modified protein or fusion protein.

The present invention also relates to recombinant vectors and recombinant host cells, both prokaryotic and eukaryotic, which contain the nucleic acid molecules disclosed throughout this specification. The synthetic polynucleotides, associated vectors, and recombinant host, cells of the present invention are useful for the production of polynucleotide vaccines described herein. In a further embodiment, an expression vector containing a variant HCMV pp65-, IE1-, or IE2-encoding nucleic acid molecule, or a nucleic acid molecule encoding a fusion protein comprising one or more of these proteins, may be used for high-level expression of said proteins in a recombinant host cell. The recombinant vectors comprise the synthetic polynucleotides disclosed throughout this specification. These vectors may be comprised of DNA or RNA. For most cloning purposes, DNA vectors are preferred. Typical vectors include plasmids, modified viruses, baculovirus, bacteriophage, cosmids, yeast artificial chromosomes, and other forms of episomal or integrated DNA that can encode the variant HCMV pp65, IE1, and IE2 proteins, or fusion proteins thereof, disclosed herein. Preferably, the expression vector also contains an origin of replication for autonomous replication in a host cell, a selectable marker, a limited number of useful restriction enzyme sites and a potential for high copy number.

The present invention also relates to host cells transformed or transfected with vectors comprising the nucleic acid molecules of the present invention, in effect serving as a factory for the modified proteins disclosed herein. The recombinant expression vector provides a recombinant polynucleotide encoding the modified protein that exists autonomously from the host cell genome or as part of the host cell genome. Recombinant host cells may be prokaryotic or eukaryotic, including but not limited to, bacteria such as E. coli, fungal cells such as yeast, mammalian cells including, but not limited to, cell lines of bovine, porcine, monkey and rodent origin; and insect cells including but not limited to Drosophila and silkworm derived cell lines. Such recombinant host cells can be cultured under suitable conditions to produce a protein or a biologically equivalent form. In an embodiment of the present invention, the host cell is human. As defined herein, the term “host cell” is not intended to include a host cell in the body of a transgenic human being, human fetus, or human embryo.

Accordingly, the polynucleotides described herein can be assembled into an expression cassette which, in turn, is inserted into a vector to be used as vaccine. The expression cassette comprises sequences designed to provide for efficient expression of the protein in a human cell. The cassette preferably contains the encoding recombinant gene, with related transcriptional and translations control sequences operatively linked to it, such as a promoter for RNA polymerase transcription and a transcription termination sequence 3′ to the recombinant gene coding sequence. In one embodiment, the promoter is the cytomegalovirus promoter with intron A sequence (CMV), although those skilled in the art will recognize that any of a number of other known promoters such as a strong immunoglobulin or other eukaryotic gene promoter may be used. Additional examples of promoters include naturally occurring promoters such as the EF1 alpha promoter, Rous sarcoma virus promoter, and SV40 early/late promoters and the p-actin promoter; and artificial promoters such as a synthetic muscle specific promoter and a chimeric muscle-specific/CMV promoter (Li et al., Nat. Biotechnol. 17:241-245 (1999); Hagstrom et al., Blood 95:2536-2542 (2000)). The synthetic genes of the present invention would be linked to such a promoter. In one embodiment, the transcriptional terminator is the bovine growth hormone (BGH) terminator, although other known transcriptional terminators may also be used. A further embodiment uses a combination of the CMV promoter and BGH terminator.

In accordance with this invention, the expression cassette may be inserted into a vector. Examples of vectors include, but not limited to, adenovirus, DNA plasmid, linear DNA or RNA linked to a promoter, adeno-associated virus, a viral vector based on herpes simplex virus, a poxvirus vector such as modified vaccinia virus Ankara, retroviral or lentiviral vector, and alphavirus vector.

In one embodiment of the invention, the vaccine vector is a DNA expression vector. DNA expression vectors are known in the art, as exemplified in US Publication No. US 2004/0087521, hereby incorporated by reference. An embodiment regarding DNA vector backbones relates to plasmid V1J (see US Publication No. US 2004/0087521). The backbone of V1J is provided by pUC18, known to produce high yields of plasmid, is well-characterized by sequence and function, and is of minimum size. V1J contains the CMVintA promoter and BGH transcription termination elements which control the expression of the recombinant genes enclosed therein. An example of a suitable plasmid would be the mammalian expression plasmid V1Jns (SEQ ID NO:28), as described in J. Shiver et. al. in DNA Vaccines, M. Liu et al. eds., N.Y. Acad. Sci., N.Y., 772:198-208 (1996), which is herein incorporated by reference. V1Jns is the same as V1J except that a unique Sfi1 restriction site has been engineered into the single Kpn1 site of V1J. The incidence of Sfi1 sites in human genomic DNA is very low (approximately 1 site per 100,000 bases). Thus, this vector allows careful monitoring for expression vector integration into host DNA, simply by Sfi1 digestion of extracted genomic DNA. It will be evidence to one of skill in the art that numerous plasmid vector constructs may be generated.

Accordingly, the present invention relates to a vaccine plasmid comprising a plasmid portion and an expression cassette portion, the expression cassette portion comprising: (a) a sequence of nucleotides (i.e., a polynucleotide) that encodes a variant HCMV pp65, IE1, or IE2 protein, or fusion protein thereof, as described herein, wherein the fusion protein is capable of producing an immune response in a mammal; and, (b) a promoter operably linked to the polynucleotide.

In another embodiment of the invention, the vector is an adenovirus vector (used interchangeably herein with “adenovector”). Adenovectors can be based on different adenovirus serotypes such as those found in humans or animals. Examples of animal adenoviruses include bovine, porcine, chimp, murine, canine and avian (CELO). In one embodiment, adenovectors are based on human serotypes, including Group 13, C, or D serotypes. Examples of human adenovirus Group B, C, D, or E serotypes include serotypes 2 (“Ad2”), 4 (“Ad4”), 5 (“Ad5”), 6 (“Ad6”), 24 (“Ad24”), 26 (“Ad26”), 34 (“Ad34”) and 35 (“Ad35”). In another embodiment, the expression vector is a human adenovirus serotype 6 (Ad6) vector.

If the vector chosen is an adenovirus, it is preferred that the vector be a so-called first-generation adenoviral vector. These adenoviral vectors are characterized by having a non-functional E1 gene region, and preferably a deleted adenoviral E1 gene region. In addition, first generation vectors may have a non-functional or deleted E3 gene region (Danthinne et al., 2000, Gene Therapy 7:1707-1714; Graham 2000, Immunology Today 21 (9):426-428). Adenovectors do not need to have their E1 and E3 regions completely removed. Rather, a sufficient amount of the E1 region is removed to render the vector replication incompetent in the absence of the E1 proteins being supplied in trans; and the E1 deletion, or the combination of the E1 and E3 deletions, is sufficiently large enough to accommodate a gene expression cassette.

In some embodiments, the expression cassette is inserted in the position where the adenoviral E1 gene is normally located. In addition, these vectors optionally have a non-functional or deleted E3 region. It is preferred that the adenovirus genome used be deleted of both the E1 and E3 regions (ΔE1ΔE3). The adenoviruses can be multiplied in known cell lines which express the viral E1 gene, such as 293 cells, or PER.C6 cells, or in cell lines derived from 293 or PER.C6 cell which are transiently or stably transformed to express an extra protein. For example, when using constructs that have a controlled gene expression, such as a tetracycline regulatable promoter system, the cell line may express components involved in the regulatory system. One example of such a cell line is T-Rex-293; others are known in the art.

For convenience in manipulating the adenoviral vector, the adenovirus may be in a shuttle plasmid form. This invention is also directed to a shuttle plasmid vector which comprises a plasmid portion and an adenovirus portion, the adenovirus portion comprising an adenoviral genome which has a deleted E1 and an optional E3 deletion, and has an inserted expression cassette comprising a recombinant HCMV gene of the present invention. In one embodiment, there is a restriction site flanking the adenoviral portion of the plasmid so that the adenoviral vector can easily be removed. The shuttle plasmid may be replicated in prokaryotic cells or eukaryotic cells.

In one embodiment of the invention exemplified in the present application, an expression cassette comprising a recombinant polynucleotide encoding a CMV protein derivative described herein is inserted into an Ad6 (ΔE1 or ΔE1ΔE3) adenovirus plasmid (see Example 3, infra; and Emini et al., US20040247615, which is hereby incorporated by reference). This vector comprises an Ad6 adenoviral genome deleted of the E1 and E3 regions. In another embodiment of the invention exemplified herein, the expression cassette is inserted into the pMRKAd5-HV0 adenovirus plasmid (see Example 3, infra; and Emini et al., US20030044421, which is hereby incorporated by reference). This plasmid comprises an Ad5 adenoviral genome deleted of the E1 and E3 regions. The design of the pMRKAd5-HV0 plasmid was improved over prior adenovectors by extending the 5′ cis-acting packaging region further into the E1 gene to incorporate elements found to be important in optimizing viral packaging, resulting in enhanced virus amplification. Advantageously, these enhanced adenoviral vectors are capable of maintaining genetic stability following high passage propagation.

Accordingly, the present invention relates to an adenoviral vaccine comprising a adenoviral portion and an expression cassette portion, the expression cassette portion comprising: (a) a sequence of nucleotides (i.e., a polynucleotide) that encodes a variant HCMV pp65, IE1, or IE2 protein, or fusion protein thereof, as described herein, wherein the fusion protein is capable of producing an immune response in a mammal; and, (b) a promoter operably linked to the polynucleotide.

Standard techniques of molecular biology for preparing and purifying DNA constructs enable the preparation of the adenoviruses, shuttle plasmids, and DNA immunogens of this invention.

One aspect of the instant invention is a method of protecting against or treating HCMV infection comprising administering to a mammal a vaccine vector which comprises a polynucleotide comprising a sequence of nucleotides that encodes a variant HCMV pp65, IE1, or IE2 protein, or fusion protein thereof, as described in the present application. In a preferred embodiment of the invention, the mammal is a human.

In one embodiment, the vector used in the methods described is an adenovirus vector or a plasmid vector. In another embodiment of the invention, the vector is an adenoviral vector comprising an adenoviral genome with a deletion in the adenovirus E1 region, and an insert in the adenovirus E1 region, wherein the insert comprises an expression cassette comprising: (a) a sequence of nucleotides (i.e., a polynucleotide) that encodes a variant HCMV pp65, IE1, or IE2 protein, or fusion protein thereof, as described herein, wherein the protein is capable of producing an immune response in a mammal; and, (b) a promoter operably linked to the polynucleotide.

In one embodiment of this aspect of the invention, the adenovirus vector is an Ad 6 vector. In another embodiment of the invention, the adenovirus vector is an Ad 5 vector. In yet another embodiment, the adenovirus vector is an Ad 24 vector. Also contemplated for use in the present invention is an adenovirus vaccine vector comprising an adenovirus genome that naturally infects a species other than human, including, but not limited to, chimpanzee adenoviral vectors. One embodiment of this aspect of the invention is a chimp Ad 3 vaccine vector.

In some embodiments of this invention, the recombinant adenovirus and plasmid-based polynucleotide vaccines disclosed herein are used in various prime/boost combinations in order to induce an enhanced immune response. In this case, the two vectors are administered in a “prime and boost” regimen. For example the first type of vector is administered one or more times, then after a predetermined amount of time, for example, 2 weeks, 1 month, 2 months, six months, or other appropriate interval, a second type of vector is administered one or more times. In one embodiment, the vectors carry expression cassettes encoding the same polynucleotide or combination of polynucleotides.

An adenoviral vector vaccine and a plasmid vaccine may be administered to a mammal as part of a single therapeutic regime to induce an immune response. To this end, the present invention relates to a method of protecting a mammal from CMV infection comprising: (a) introducing into the mammal a first vector comprising: i) a sequence of nucleotides (i.e., a polynucleotide) that encodes a variant HCMV pp65, IE1, or IE2 protein, or fusion protein thereof, as described herein, wherein the protein is capable of producing an immune response in a mammal; and, ii) a promoter operably linked to the polynucleotide; (b) allowing a predetermined amount of time to pass; and, (c) introducing into the mammal a second vector comprising: i) a sequence of nucleotides (i.e., a polynucleotide) that encodes a variant HCMV pp65, IE1, or IE2 protein, or fusion protein thereof, as described herein, wherein the protein is capable of producing an immune response in a mammal; and, ii) a promoter operably linked to the polynucleotide.

In one embodiment of the method of protection described above, the first vector is a plasmid and the second vector is an adenovirus vector. In an alternative embodiment, the first vector is an adenovirus vector and the second vector is a plasmid. In some embodiments of the present invention, the first vector is administered to the patient more than one time before the second vector is administered. In another embodiment, both the first and second vector is an adenovirus vector, wherein the first and second adenovirus vectors are derived from different serotypes.

In the method described above, the first type of vector may be administered more than once, with each administration of the vector separated by a predetermined amount of time. Such a series of administration of the first type of vector may be followed by administration of a second type of vector one or more times, after a predetermined amount of time has passed. Similar to treatment with the first type of vector, the second type of vector may also be given one time or more than once, following predetermined intervals of time.

The instant invention further relates to a method of treating a mammal (i.e., a mammalian patient) suffering from a HCMV infection comprising: (a) introducing into the mammal a first vector comprising: i) a sequence of nucleotides (i.e., a polynucleotide) that encodes a variant HCMV pp65, IE1, or IE2 protein, or fusion protein thereof, as described herein, wherein the protein is capable of producing an immune response in a mammal; and, ii) a promoter operably linked to the polynucleotide; (b) allowing a predetermined amount of time to pass; and (c) introducing into the patient a second vector comprising: i) a sequence of nucleotides (i.e., a polynucleotide) that encodes a variant HCMV pp65, IE1, or IE2 protein, or fusion protein thereof, as described herein, wherein the protein is capable of producing an immune response in a mammal; and, ii) a promoter operably linked to the polynucleotide.

In one embodiment of the method of treatment described above, the first vector is a plasmid and the second vector is an adenovirus vector. In an alternative embodiment, the first vector is an adenovirus vector and the second vector is a plasmid. In further preferred embodiments of the method described above, the first vector is administered to the patient more than one time before the second vector is administered to the patient. In another embodiment, both the first and second vector is an adenovirus vector, wherein the first and second adenovirus vectors are derived from different serotypes.

The amount of expressible DNA or transcribed RNA to be introduced into a vaccine recipient will depend partially on the strength of the promoters used and on the immunogenicity of the expressed gene product. In general, an immunologically or prophylactically effective dose of about 1 ng to 100 mg, and preferably about 10 μg to 300 μg of a plasmid vaccine vector is administered directly into muscle tissue. An effective dose for recombinant adenovirus is approximately 10⁶-10¹² particles and preferably about 10⁷-10¹¹ particles. Subcutaneous injection, intradermal introduction, impression through the skin, and other modes of administration such as intraperitoneal, intravenous, intramuscular or inhalation delivery are also contemplated. In one embodiment of the present invention, the vaccine vectors are introduced to the recipient through intramuscular injection.

The vaccine vectors of the present invention may be formulated in a pharmaceutically effective formulation for host administration. The vaccine vectors of this invention may be naked, i.e., unassociated with any proteins, or other agents which impact on the recipient's immune system. In this case, it is desirable for the vaccine vectors to be comprised within a pharmaceutical composition further comprising a physiologically acceptable solution, such as, but not limited to, sterile saline or sterile buffered saline (e.g., PBS).

It will be useful to utilize pharmaceutically acceptable formulations which also provide long-term stability of the vaccine vectors of the present invention. For example, during storage as a pharmaceutical entity, plasmid vaccines undergo a physiochemical change in which the supercoiled plasmid converts to the open circular and linear form. A variety of storage conditions (e.g., low pH, high temperature, low ionic strength) can accelerate this process. Therefore, the removal and/or chelation of trace metal ions (with succinic or malic acid, or with chelators containing multiple phosphate ligands) from the plasmid solution, from the formulation buffers or from the vials and closures, stabilizes the DNA plasmid from this degradation pathway during storage. In addition, inclusion of non-reducing free radical scavengers, such as ethanol or glycerol, is useful to prevent damage of the DNA plasmid from free radical production that may still occur. Furthermore, the buffer type, pH, salt concentration, light exposure, as well as the type of sterilization process used to prepare the vials, may be controlled in the formulation to optimize the stability of the DNA vaccine. Therefore, formulations that will provide the highest stability of the plasmid vaccine will be one that includes a demetalated solution containing a buffer (phosphate or bicarbonate) with a pH in the range of 7-8, a salt (NaCl, KCl, or LiCl) in the range of 100-200 mM, a metal ion chelator (e.g., EDTA, diethylenetriaminepenta-acetic acid (DTPA), malate, inositol hexaphosphate, tripolyphosphate, or polyphosphoric acid), a non-reducing free radical scavenger (e.g., ethanol, glycerol, methionine, or dimethyl sulfoxide) and the highest appropriate DNA concentration in a sterile glass vial, packaged to protect the highly purified, nuclease free DNA from light. The use of stabilized plasmid vector vaccines and formulations thereof is described in US Publication No. US 2002/0156037, which is hereby incorporated by reference.

Alternatively, it may be advantageous to administer an agent which assists in the cellular uptake of DNA, such as, but not limited to calcium ion. These agents are generally referred to as transfection facilitating reagents and pharmaceutically acceptable carriers. Those of skill in the art will be able to determine the particular reagent or pharmaceutically acceptable carrier as well as the appropriate time and mode of administration.

The polynucleotide vector vaccines of the present invention may, in addition to generating a strong cell-mediated immune response, provide for a measurable humoral response subsequent to immunization. This response may occur with or without the addition of an adjuvant to the respective vaccine formulation. To this end, the polynucleotide vector vaccines of the present invention may also be formulated with an adjuvant or adjuvants which may increase immunogenicity of the vaccines. Adjuvants are particularly useful for DNA plasmid vaccines. Examples of adjuvants are toll-like receptor agonists, alum, AlPO4, alhydrogel, Lipid-A and derivatives or variants thereof, Freund's incomplete adjuvant, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers may be used as an adjuvant (Newman et al., 1998, Critical Reviews in Therapeutic Drug Carrier Systems 15:89-142). The immune response of a nucleic acid can be enhanced using a non-ionic block copolymer combined with an anionic surfactant.

Polynucleotides encoding variant HCMV pp65, IE1, IE2 proteins, fusion proteins thereof, and the encoded proteins, described herein can elicit an immune response against HCMV. A CMI immune response can be generated against one or more regions containing human MHC-restricted T-cell epitopes present in the wild-type HCMV sequence. Examples of known pp65 and IE1 T-cell epitopes are provided in Tables 2 and 3, and the references cited in these tables. Known T-cell epitopes can be used as a guide to produce different polypeptides maintaining most T-cell epitopes (e.g., at least 80%, at least 90, or at least 95%).

TABLE 2
Known human T cell Epitopes to HCMV pp65

| Amino | | HLA | |
|---|---|---|---|
| acids | Peptide | allele | Reference |
| 14-22 | VLGPISGHV | A2 | Solache et al, 1999, The Journal of Immunology, |
| | SEQ ID NO: 30 | | 163:5512 |
| 123-131 | IPSINVHHY | B35 | Hassan-Walker et al, 2001, Journal of Infectious |
| | SEQ ID NO: 31 | | Disease, 183:835 |
| 369-337 | FTSQYRIQGKL | A24 | Longmate et al, 2001, Immunogenetics, 52:165 |
| | SEQ ID NO: 32 | | |
| 490-498 | ILARNLVPM | A2 | Elkington et al, 2003, Journal of Virology 77:5226 |
| | SEQ ID NO: 33 | | |
| 495-503 | NLVPMVATV | A2 | Gillespieet al, 2000, Journal of Virology, 74:8140 |
| | SEQ ID NO: 34 | | |
| 512-521 | EFFWDANDIY | B44 | Wills et al, 2002, The Journal of Immunology, |
| | SEQ ID NO: 35 | | 168:5455 |
| 41-55 | LLQTGIHVRVSQPSL | DR15 | Kern et al, 2002, Journal of Infectious Disease, |
| | SEQ ID NO: 36 | | 185:1709 |
| 445-459 | ACTSGVMTRGRLKAE | DR1 | Li Pira et al, 2004, Int. Immunol., 16:635 |
| | SEQ ID NO: 37 | | |

The indicated amino acid regions are with respect to the wild-type sequence.

TABLE 3
Known human T Cell Epitopes to HCMV IE1

| Amino | | HLA | |
|---|---|---|---|
| acids | Peptide | allele | Reference |
| 81-89 | VLAELVKQI | A2 | Elkington et al, 2003, Journal of Virology |
| | SEQ ID NO: 38 | | 77:5226 |
| 88-96 | QIKVRVDMV | B8 | Elkington et al, 2003, Journal of Virology |
| | SEQ ID NO: 39 | | 77:5226 |
| 198-207 | DELRRKMMYM | B8 | Wills et al, 2002, The Journal of |
| | SEQ ID NO: 40 | | Immunology, 168:5455 |
| 279-287 | CVETMCNEY | B18 | Retiere et al, 2000, Journal of Virology, |
| | SEQ ID NO: 41 | | 74:3948 |
| 316-324 | VLEETSVML | A2 | Khan et al, 2002, Journal of Infectious |
| | SEQ ID NO: 42 | | Disease, 185:1025 |
| 91-110 | VRVDMVRHRIKEHMLKKYTQ | DR3 | Davignon et al, 1996, Journal of Virology, |
| | SEQ ID NO: 43 | | 70:2162 |
| 162-175 | DKREMWMACIKELH | DR8 | Gautier et al, 1996, Eur. J. Immunol., |
| | SEQ ID NO: 44 | | 26:1110 |

The indicated amino acid regions are with respect to the wild-type sequence.

In different embodiments described herein related to a variant pp65 encoding sequence or the polypeptide itself, the variant pp65 comprises or consists of a sequence substantially similar to SEQ ID NO: 1 or 3 containing one or modifications described herein and maintaining most T-cell epitopes provided in the wild-type sequence.

In further embodiments the variant pp65 sequence is substantially similar to SEQ ID NOs: 1 or 3 and contain at least 4, 5, 6, 7 or 8 T-cell epitopes provided in Table 2. Such sequences preferably also have an overall sequence identity to SEQ ID NO: 1 or 3 of at least 75%, at least 85%, at least 90%, at least 95%, or at least 99%; or contain 0, 1, 2, 3, 4, 5, 6, 7, 8, 9, 10, 11, 12, 13, 14, 15, 16, 17, 18, 19, or 20 amino acids alterations from SEQ ID NOs: 3; or contain 1, 2, 3, 4, 5, 6, 7, 8, 9, 10, 11, 12, 13, 14, 15, 16, 17, 18, 19, or 20 amino acids alterations from SEQ ID NO: 1. Possible changes to sequence identity or amino acid alterations do not occur in particular amino acids that are specifically recited as part of a variant pp65 sequence (e.g., amino acids recited to reduce NLS activity), or result in providing for activity specifically indicated to be decreased (e.g., reduced NLS activity).

The number of T-cells epitopes can vary independent of the sequence similarity or amino acid alterations. Thus, any combination of the number of T-cell epitopes can be combined with amino acid differences. Examples include 8 T-cell epitopes with a 95% sequence identity, 8 T-cell epitopes with 20 amino acid alterations, 7 T-cell epitopes with a 95% sequence identity, 7 T-cell epitopes with 20 amino acid alterations and so on, where the T-cell epitopes are proved in Table 2.

In different embodiments described herein related to a variant IE1 encoding sequence or the polypeptide itself, the variant IE1 comprises or consists of a sequence substantially similar to SEQ ID NOs: 6 or 9, containing one or modifications described herein, wherein most T-cell epitopes from the wild-type sequence are retained.

In further embodiments the variant IE1 is sequence is substantially similar to SEQ ID NOs: 6 or 9 and contain at least 4, 5, 6, or 7 T-cell epitopes provided in Table 3. Such sequences preferably also have an overall sequence identity to SEQ ID NO: 6 or 9 of at least 75%, at least 85%, at least 90%, at least 95%, or at least 99%; or contain 0, 1, 2, 3, 4, 5, 6, 7, 8, 9, 10, 11, 12, 13, 14, 15, 16, 17, 18, 19, or 20 amino acids alterations from SEQ ID NO: 9; or contain 1, 2, 3, 4, 5, 6, 7, 8, 9, 10, 11, 12, 13, 14, 15, 16, 17, 18, 19, or 20 amino acids alterations from SEQ ID NO: 6. Possible changes to sequence identity or amino acid alterations do not occur in particular amino acids that are specifically recited as part of a modified IE1 sequence (e.g., amino acids recited to reduce NLS activity), or result in variant providing for activity specifically indicated to be decreased (e.g., reduced NLS activity).

The number of T-cells epitopes can vary independent of the sequence similarity. Thus, any combination of the number of T-cell epitopes can be combined with amino acid differences. Examples include 7 T-cell epitopes with a 95% sequence identity, 7 T-cell epitopes with 20 amino acid alterations, 6 T-cell epitopes with a 95% sequence identity, 6 T-cell epitopes with 20 amino acid alterations and so on, where the T-cell epitopes are proved in Table 3.

The embodiment described above referencing T-cell epitopes also apply to descriptions of variant pp65 and/or IE1 present in a fusion protein, and the encoding nucleic acid.

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

The following examples further illustrate, but do not limit the invention.

### Example 1

### Selection of CMV Antigens

ELISPOT assay—The method for IFN-γ ELISPOT assay was published previously (Fu et al, 2007, AIDS Res Human Retrovirus. 23:67). Briefly, 96-well microtiter plates with PVDF membrane (Millipore, Bedford, Mass.) were coated with mouse anti-human IFN-γ mAb clone 1-D1K (MabTech, Stockholm, Sweden) at 10 μg/ml. Coated plates were washed and blocked 2 hours with complete RPMI-1640 medium supplemented with 10% fetal bovine serum (R-10, Gibco-BRL, Grand Island, N.Y.). Blocking buffer was removed and 100 μl/well of PBMC diluted in R10 were added to result in 2×10⁵ and 1×10⁵ cells/well. Antigen (peptide pools or viral lysate) was diluted in R10 and added at 25 μl/well, and the final concentration for each peptide in the pools was about 2 μg/ml. Peptide-free DMSO diluent matching the DMSO concentration in the peptide solutions was used as a negative control (mock antigen). Plates were incubated overnight in a humidified CO₂ incubator at 37° C. and washed with PBS containing 0.05% Tween 20. Biotinylated anti-human IFN-γ monoclonal antibody clone 7-B6-1 (MabTech) at 1 μg/ml was added to the plates and incubated 2-4 hours at room temperature. Plates were washed with PBS/Tween and 100 μl/well of alkaline phosphatase-conjugated anti-biotin monoclonal antibody (Vector Laboratories, Burlingame, Calif.) at 1:750 in assay diluent was added to each well. Plates were incubated 2 hours at room temperature and washed with PBS/Tween. To develop the spots, 100 μl/well of precipitating alkaline phosphatase substrate NBT/BCIP (Pierce, Rockford, Ill.) was added to each well and incubated at room temperature until spots became visible (usually 5-10 minutes). The number of spots per well was normalized to per 1×10⁶ cells and averaged for each sample and antigen.

Antigens selected for target were chosen based on one or more of the following criteria: (a) present in immediate early (IE) stages of the viral replication cycle; (b) considered either a major viral antigen, a major component in viral particles or abundantly expressed in the IE phase of viral life cycle; (c) essential or important for viral replication; and, (d) has the ability to elicit T-cell responses in CMV infected human subjects. Based on these criteria, pp65, IE1 and IE2 were selected as antigens for inclusion in a developmental CMV vaccine. Table 4 summarizes the criteria used to select pp65, IE1 and IE2.

TABLE 4
Properties of selected CMV antigens.

| | | Size | Essential | Content in | Responder |
|---|---|---|---|---|---|
| | Antigen | (amino | in viral | purified | frequency |
| | (gene name) | acids) | life cycle¹ | virions (%)² | (%)³ |
| Tegument/ | pp65 (UL83) | 561 | No | 15.4 | CD4: 75 |
| structural | | | | | CD8: 55 |
| protein | | | | | |
| Immediate | IE1 (UL123) | 491 | Augment | Minimal | CD4: 33 |
| early | | | | | CD8: 55 |
| antigen | IE2 (UL122) | 579 | Yes | Minimal | CD4: 49 |
| | | | | | CD8: 36 |

¹Yu et al, 2003, Proc. Nat'l Acad. Sci. USA 100: 12396-12401.
²Varnum et al, 2004, J. Virol. 78: 10960.
³Sylwester et al, 2005, J. Exp. Med. 202: 673.

To confirm that these antigens are indeed immunogenic in humans, both seropositive (n=40) and seronegative human (n=10) subjects were screened for T-cell responses against the CMV antigens. Samples of peripheral blood mononuclear cells (PBMCs) were collected and evaluated in human IFN-γ ELISPOT assays. The antigens evaluated include peptide pools of 15-mer peptides overlapping by 11 amino acids corresponding to the ORFS of pp65, IE1, IE2, and gB. CMV infected and mock-infected MRC-5 cell lysates were also included as controls. CMV-infected MRC-5 cell lysates contained a multitude of HCMV antigens. As expected, PBMCs from CMV seropositive donors responded to the CMV antigens, antigen peptide pools (IE1, IE2, pp65, and gB), and HCMV infected MRC-5 lysates, but not to the mock peptide pool or mock infected lysate. A positive ELISPOT response was scored as greater than 55 SFC/10⁶ PBMC and greater than 4 fold rise over mock antigen (Fu et al, 2007, supra). The responder rates to IE1, IE2, pp65, and gB were thus determined to be 55%, 28%, 90%, and 78%, respectively. There were no ELISPOT responses from CMV seronegative subjects. This result is in line with a previous study on 33 human subjects, summarized in Table 4, using intracellular staining method (Sylwester et al, 2005, J. Exp. Med. 202:673).

### Example 2

### Functional Inactivation Strategies for CMV pp65, IE1 and IE2

DNA sequences corresponding to HCMV antigens of interest were generated either by PCR amplification of viral genomic DNA (e.g., pp65 ORF) or by custom synthesis (e.g., IE1; IE2, mpp65).

pp65—Viral protein pp65 (UL83), also called lower matrix protein, is a major tegument protein of 561 amino acids. It accounts for over 15% of the total viral proteins by mass in purified CMV virions (Varnum et al, 2004, J. Virol. 78:10960-10966). It contains casein kinase II phosphorylation sites (residues 426-498) and displays serine/threonine kinase activity in vitro (Somogyi et al, 1990, Virol. 174:276-285). A carboxyl fragment of 173 amino acids contains a putative kinase domain of ATP binding motifs with a highly conserved lysine at residue 436. In addition, pp65 contains a bipartite nuclear localization signal (NLS) (Gallina et al, 1996, J. Gen. Virol. 77:1151-1157; Schmolke et al, 1995, J. Virol. 69:1071-1078).

The strategy to inactivate pp65 function includes deletion and/or modification of the bipartite NLS (Gallina et al, 1996, J. Gen. Virol. 77:1151-1157; Schmolke et al, 1995, J. Viral. 69:1071-1078). In addition, a substitution of the conserved lysine at position 436 with a glycine to nullify the protein kinase activity was incorporated into the sequence. A report has shown that the ability of pp65 to phosphorylate casein substrate in vitro can be abrogated with a single point mutation at residue 436 (Yao et al, 2001, Vaccine 19:1628-1635).

The wildtype amino acid sequence for human CMV pp65, designated herein as “pp65,” is set forth as SEQ ID NO:1:

| (SEQ ID NO: 1) | |
|---|---|
| 1 | MESRGRRCPE MISVLGPISG HVLKAVFSRG DTPVLPHETR LLQTGIHVRV |
| 51 | SQPSLILVSQ YTPDSTPCHR GDNQLQVQHT YFTGSEVENV SVNVHNPTGR |
| 101 | SICPSQEPMS IYVYALPLKM LNIPSINVHH YPSAAERKHR HLPVADAVIH |
| 151 | ASGKQMWQAR LTVSGLAWTR QQNQWKEPDV YYTSAFVFPT KDVALRHVVC |
| 201 | AHELVCSMEN TRATKMQVIG DQYVKVYLES FCEDVPSGKL FMHVTLGSDV |
| 251 | EEDLTMTRNP QPFMRPHERN GFTVLCPKNM IIKPGKISHI MLDVAFTSHE |
| 301 | HFGLLCPKSI PGLSISGNLL MNGQQIFLEV QAIRETVELR QYDPVAALFF |
| 351 | FDIDLLLQRG PQYSEHPTFT SQYRIQGKLE YRHTWDRHDE GAAQGDDDVW |
| 401 | TSGSDSDEEL VTTERKTPRV TGGGAMAGAS TSAGRKRKSA SSATACTSGV |
| 451 | MTRGRLKAES TVAPEEDTDE DSDNEIHNPA VFTWPPWQAG ILARNLVPMV |
| 501 | ATVQGQNLKY QEFFWDANDI YRIFAELEGV WQPAAQPKRR RHRQDALPGP |
| 551 | CIASTPKKHR G. |

The two nuclear localization sequences (NLSs) are underlined: NLS1 (amino acids 415-438) and NLS2 (amino acids 537-561). Wild-type pp65 is encoded by the nucleic acid sequence as set forth in SEQ ID NO:2 (“pp65 (nuc)”). The amino acid and encoding nucleotide sequence of wild-type pp65 are also disclosed in NCBI Accession nos. P06725 and NC_{—}001347 (nucleotides 120283-121968), respectively.

The amino acid sequence of a modified pp65 protein, designated herein as “mpp65,” is set forth as SEQ ID NO:3:

| (SEQ ID NO: 3) | |
|---|---|
| 1 | MESRGRRCPE MISVLGPISG HVLKAVFSRG DTPVLPHETR LLQTGIHVRV |
| 51 | SQPSLILVSQ YTPDSTPCHR GDNQLQVQHT YFTGSEVENV SVNVHNPTGR |
| 101 | SICPSQEPMS IYVYALPLKM LNIPSINVHH YPSAAERKHR HLPVADAVIH |
| 151 | ASGKQMWQAR LTVSGLAWTR QQNQWKEPDV YYTSAFVFPT KDVALRHVVC |
| 201 | AHELVCSMEN TRATKMQVIG DQYVKVYLES FCEDVPSGKL FMHVTLGSDV |
| 251 | EEDLTMTRNP QPFMRPHERN GFTVLCPKNM IIKPGKISHI MLDVAFTSHE |
| 301 | HFGLLCPKSI PGLSISGNLL MNGQQIFLEV QAIRETVELR QYDPVAALFF |
| 351 | FDIDLLLQRG PQYSEHPTFT SQYRIQGKLE YRHTWDRHDE GAAQGDDDVW |
| 401 | TSGSDSDEEL VTTEGGTPGV TGGGAMAGAS TSAGRGRKSA SSATACTSGV |
| 451 | MTRGRLKAES TVAPEEDTDE DSDNEIHNPA VFTWPPWQAG ILARNLVPMV |
| 501 | ATVQGQNLKY QEFFWDANDI YRIFAELEGV WQPAA. |

mpp65 has a modification in the NLS1 region consisting of the following amino acid substitutions: R415G, K416G and R419G (underlined above in SEQ ID NO:3). NLS2 has been removed by a COOH-terminal truncation of the wild-type protein, starting at amino acid residue 536 of pp65. The putative, protein kinase activity is also removed by a single amino acid substitution, K436G (underlined above).

The nucleic acid sequence that encodes mpp65, designated herein as “mpp65 (nuc),” is set forth as SEQ ID NO:4:

(SEQ ID NO: 4)
ATGGAGTCGCGCGGTCGCCGTTGTCCCGAAATGATATCCGTACTGGGT
CCCATTTCGGGGCACGTGCTGAAAGCCGTGTTTAGTGGCGGCGATACG
CCGGTGCTGCCGCACGAGACGCGACTCCTGCAGACGGGTATCCACGTA
CGCGTGAGCCAGCCCTCGCTGATCTTGGTATCGCAGTACACGCCCGAC
TCGACGCCATGCCACCGCGGCGACAATCAGCTGCAGGTGCAGCACACG
TACTTTACGGGCAGCGAGGTGGAGAACGTGTCGGTCAACGTGCACAAC
CCCACGGGCCGAAGCATCTGCCCCAGCCAGGAGCCCATGTCGATCTAT
GTGTACGCGCTGCCGCTCAAGATGCTGAACATCCCCAGCATCAACGTG
CACCACTACCCGTCGGCGGCCGAGCGCAAACACCGACACCTGCCCGTA
GCTGACGCTGTGATTCACGCGTCGGGCAAGCAGATGTGGCAGGCGCGT
CTCACGGTCTCGGGACTGGCCTGGACGCGTCAGCAGAACCAGTGGAAA
GAGCCCGACGTCTACTACACGTCAGCGTTCGTGTTTCCCACCAAGGAC
GTGGCACTGCGGCACGTGGTGTGCGCGCACGAGCTGGTTTGCTCCATG
GAGAACACGCGCGCAACCAAGATGCAGGTGATAGGTGACCAGTACGTC
AAGGTGTACCTGGAGTCCTTCTGCGAGGACGTGCCCTCCGGCAAGCTC
TTTATGCACGTCACGCTGGGCTCTGACGTGGAAGAGGACCTGACGATG
ACCCGCAACCCGCAACCCTTCATGCGCCCCCACGAGCGCAACGGCTTT
ACGGTGTTGTGTCCCAAAAATATGATAATCAAACCGGGCAAGATCTCG
CACATCATGCTGGATGTGGCTTTTACCTCACACGAGCATTTTGGGCTG
CTGTGTCCCAAGAGCATCCCGGGCCTGAGCATCTCAGGTAACCTGTTG
ATGAACGGGCAGCAGATCTTCCTGGAGGTACAAGCCATACGCGAGACC
GTGGAACTGCGTCAGTACGATCCCGTGGCTGCGCTCTTCTTTTTCGAT
ATCGACTTGCTGCTGCAGCGCGGGCCTCAGTACAGCGAGCACCCCACC
TTCACCAGCCAGTATCGCATCCAGGGCAAGCTTGAGTACCGACACACC
TGGGACCGGCACGACGAGGGTGCCGCCCAGGGCGACGACGACGTCTGG
ACCAGCGGATCGGACTCCGACGAAGAACTCGTAACCACCGAGGGCGGG
ACGCCCGGCGTCACCGGCGGCGGCGCCATGGCGGGCGCCTCCACTTCC
GCGGGCCGCGGACGCAAATCAGCATCCTCGGCGACGGCGTGCACGTCG
GGCGTTATGACACGCGGCCGCCTTAAGGCCGAGTCCACCGTCGCGCCC
GAAGAGGACACCGACGAGGATTCCGACAACGAAATCCACAATCCGGCC
GTGTTCACCTGGCCGCCCTGGCAGGCCGGCATCCTGGCCCGCAACCTG
GTGCCCATGGTGGCTACGGTTCAGGGTCAGAATCTGAAGTACCAGGAA
TTCTTCTGGGACGCCAACGACATCTACCGCATCTTCGCCGAATTGGAA
GGCGTATGGCAGCCCGCTGCG

A codon-optimized version of mpp65 (nuc), designated herein a “mpp65.syn,” is set forth in SEQ ID NO:5:

(SEQ ID NO: 5)
ATGGAGTCTCGTGGTCGTCGGTGCCCTGAGATGATCTCTGTGCTGGGA
CCCATCTCTGGCCATGTGCTGAAGGCTGTCTTCTCTCGGGGAGACACC
CCTGTGCTGCCTCATGAGACCCGGCTGCTTCAGACAGGCATCCATGTG
CGGGTCTCCCAGCCATCCCTGATCCTGGTCTCCCAGTACACCCCTGAC
TCTACCCCATGCCATCGGGGTGACAACCAGCTTCAGGTGCAGCACACC
TACTTCACAGGCTCTGAGGTGGAGAATGTCTCTGTGAATGTTCACAAC
CCTACAGGCCGGTCCATCTGCCCATCCCAGGAGCCCATGTCCATCTAT
GTCTATGCCCTGCCTCTGAAGATGCTGAACATCCCATCCATCAATGTG
CATCACTACCCATCTGCTGCTGAGCGGAAGCATCGGCATCTGCCTGTG
GCTGATGCTGTGATCCATGCCTCTGGCAAGCAGATGTGGCAGGCTCGG
CTGACAGTCTCTGGCCTGGCCTGGACTCGGCAGCAGAACCAGTGGAAG
GAGCCTGATGTCTACTACACCTCTGCCTTTGTCTTCCCCACCAAGGAT
GTGGCTCTGCGGCATGTGGTCTGTGCTCATGAGCTGGTCTGCTCTATG
GAGAACACTCGGGCCACCAAGATGCAGGTGATTGGTGACCAGTATGTG
AAGGTCTACCTGGAGTCCTTCTGTGAGGATGTGCCATCTGGCAAGCTG
TTCATGCATGTGACCCTGGGCTCTGATGTGGAGGAGGACCTGACCATG
ACTCGGAACCCTCAGCCATTCATGCGGCCTCATGAGCGGAATGGCTTC
ACAGTGCTGTGCCCTAAGAACATGATCATCAAGCCTGGCAAGATCAGC
CACATCATGCTGGATGTGGCCTTCACCTCCCATGAGCACTTTGGCCTG
CTGTGCCCCAAGTCCATCCCTGGCCTGTCCATCTCTGGCAACCTGCTG
ATGAATGGCCAGCAGATATTCCTGGAGGTGCAGGCCATCCGGGAGACA
GTGGAGCTGCGGCAGTATGACCCTGTGGCTGCTCTGTTCTTCTTTGAC
ATTGACCTGCTACTGCAGCGGGGCCCTCAGTACTCTGAGCATCCCACC
TTCACCTCCCAGTACCGTATCCAGGGCAAGCTGGAGTACCGGCACACC
TGGGACCGGCATGATGAGGGTGCTGCCCAGGGTGATGATGATGTCTGG
ACCTCTGGCTCTGACTCTGATGAGGAGCTGGTGACCACAGAGGGTGGC
ACCCCTGGTGTGACAGGTGGAGGTGCTATGGCTGGTGCCTCCACCTCT
GCTGGTCGGGGTCGGAAGTCTGCCTCCTCTGCCACAGCTTGCACCTCT
GGTGTGATGACTCGTGGTCGGCTGAAGGCTGAGTCCACAGTGGCTCCT
GAGGAGGACACAGATGAGGACTCTGACAATGAGATCCACAACCCTGCT
GTCTTCACCTGGCCTCCATGTCAGGCTGGCATCCTGGCTCGGAACCTG
GTGCCTATGGTGGCCACAGTGCAGGGTCAGAACCTGAAGTACCAGGAG
TTCTTCTGGGATGCCAATGACATCTACCGGATCTTTGCTGAGCTGGAG
GGTGTCTGTCAGCCTGCTGCC.

This sequence was constructed synthetically using Lathe codon optimization algorithms (Lathe, 1985, “Synthetic Oligonucleotide Probes Deduced from Amino Acid Sequence Data: Theoretical and Practical Considerations” J. Molec. Biol. 183:1-12).

IE1 and IE2—Expression of both viral major immediate early antigen 1 (IE1, UL123) and IE2 (UL122) is driven by the major immediate early promoter (MIEP) through alternative splicing. The IE1 transcript contains exons 1, 2, 3 and 4; and the IE2 transcript contains exons 1, 2, 3 and 5. Thus, the two proteins share the first 85 amino acids (encoded by exons 2 and 3). Both IE1 (491 amino acids) and IE2 (579 amino acids) are nuclear proteins with well-defined, bipartite NLSs (Wilkinson et al, 1998, J. Gen. Virol. 79:1233-1245; Delmas et al, 2005, J. Immunol. 175:6812-3819; Pizzorno et al, 1991, J. Virol. 65:3839-3852). They are important for viral gene regulation, with IE1 augmenting MIEP activity and IE2 inhibiting MIEP activity (Mocarski, Edward S. “Cytomegaloviruses and Their Replication.” Fields Virology, 3rd Edition. Ed. Bernard N. Fields. Lippincott Williams & Wilkins, 1996. 2447-22492; Petrik et al, 2006, J. Virol. 80:3872-3883). In addition, both proteins have been shown to modulate host cell cycles, possibly through their interactions with Rb family proteins: p107 for IE1, and p53 and Rb for IE2 (Johnson et al, 1999, J. Gen. Viral. 80:1293-1303; Hagemeier et al, 1994, EMBO J. 13:2897-2903; Hsu et al, 2004, EMBO J. 23:2269-2280; reviewed in Castillo and Kowalik, 2002, Gene 290:19-34).

The modification strategies for IE1 and IE2 include the following: 1) modification or removal of the NLSs to limit proteins to cytoplasm, thus reducing the chance of interaction with cell cycle modulation proteins, such as p53, Rb and p107, and with nuclear domain 10 (ND-10) and cellular transcriptional activation factors; and, 2) removal of exons 2 and 3 to eliminate probability of activating latent HCMV (White and Spector, 2005, J. Virol. 79:7438-7452) and interacting with cell cycle protein p107 (Johnson et al, 1999, J. Gen Virol, 80:1293). Exons 2 and 3 contain a structure that is important for binding to p107, and thus the deletion of exons 2 and 3 can remove suppression of p107 on cell proliferation (Johnson et al, 1999, supra). Furthermore, a mutant HCMV virus having a deletion in its genome corresponding to amino acids 30 to 77 of IE1 and IE2 showed severely impaired growth kinetics in fibroblast cells, even at high MOI (White and Spector, 2005, supra). The mutant virus failed to disrupt ND-10 structure, but maintained mutant IE2 accumulation. However, mutant IE2 was not fully functional in activating viral early gene expression (White and Spector, 2005, supra). In some of the mutant IE2 transcripts, two (2) point mutations were introduced at positions 446 and 452, converting histidine to alanine, which have been demonstrated to nullify ability of IE2 to negatively regulate MIEP activity and abrogate viral replication (Macias and Stinski, 1993, Proc. Nat'l Acad. Sci. USA 70:707-711; Petrik et al, 2007, J. Virol. 81:5807-5818).

The wildtype amino acid sequence for human CMV IE1, designated herein as “IE1,” is set forth as SEQ ID NO:6:

| (SEQ ID NO: 6) | |
|---|---|
| 1 | MESSAKRKMD PDNPDEGPSS KVPRPETPVT KATTFLQTML RKEVNSQLSL |
| 51 | GDPLFPELAE ESLKTFEQVT EDCNENPEKD VLAELVKQIK VRVDMVRHRI |
| 101 | KEHMLKKYTQ TEEKFTGAFN MMGGCLQNAL DILDKVHEPF EEMKCIGLTM |
| 151 | QSMYENYIVP EDKREMWMAC IKELHDVSKG AANKLGGALQ AKARAKKDEL |
| 201 | RRKMMYMCYR NIEFFTKNSA FPKTTNGCSQ AMAALQNLPQ CSPDEIMAYA |
| 251 | QKIFKILDEE RDKVLTHIDH IFMDILTTCV ETMCNEYKVT SDACMMTMYG |
| 301 | GISLLSEFCR VLCCYVLEET SVMLAKRPLI TKPEVISVMK RRIEEICMKV |
| 351 | FAQYILGADP LRVCSPSVDD LRAIAEESDE EEAIVAYTLA TAGVSSSDSL |
| 401 | VSPPESPVPA TIPLSSVIVA ENSDQEESEQ SDEEEEEGAQ EEREDTVSVK |
| 451 | SEPVSEIEEV APEEEEDGAE EPTASGGKST HPMVTRSKAD Q. |

The two NLSs of IE1 are underlined: NLS1 (amino acids 2-25) and NLS2 (amino acids 326-342). The portion of IE1 that is encoded by exon 3 spans amino acid 25-85 of SEQ ID NO:6. IE1 is encoded by the nucleic acid sequence as set forth in SEQ ID NO:7. These sequences are also disclosed in NCBI Accession nos. NP_{—}040060 (protein) and NC_{—}001347.2 (joining nucleotides 171937-173156, 173327-473511, and 173626-173696) (nucleic acid). A codon-optimized version of the nucleic acid sequence that encodes IE1, IE1.syn, and was generated using Lathe codon optimization algorithms (Lathe, 1985, supra) is set forth as SEQ ID NO:8.

(SEQ ID NO: 8)
ATGGAGTCCTCTGCCAAGCGGAAGATGGACCCTGACAACCCTGATGAG
GGCCCATCCTCCAAGGTGCCTCGGCCTGAGACCCCTGTGACCAAGGCC
ACCACCTTCCTGCAGACCATGCTGCGGAAGGAGGTGAACTCCCAGCTG
TCCCTGGGCGACCCTCTGTTCCCTGAGCTGGCTGAGGAGTCCCTGAAG
ACCTTTGAGCAGGTGACAGAGGACTGCAATGAGAACCCTGAGAAGGAT
GTGCTGGCTGAGCTGGTGAAGCAGATCAAGGTGCGGGTGGACATGGTG
CGGCATCGGATCAAGGAGCACATGCTGAAGAAGTACACCCAGACAGAG
GAGAAGTTCACAGGCGCCTTCAACATGATGGGTGGCTGCCTGCAGAAT
GCCCTGGACATCCTGGACAAGGTGCATGAGCCATTTGAGGAGATGAAG
TGCATTGGCCTGACCATGCAGTCCATGTATGAGAACTACATTGTGCCT
GAGGACAAGCGGGAGATGTGGATGGCCTGCATCAAGGAGCTGCATGAT
GTCTCCAAGGGCGCTGCCAACAAGCTGGGCGGTGCCCTGCAGGCCAAG
GCCCGGGCCAAGAAGGATGAGCTGCGGCGGAAGATGATGTACATGTGC
TACCGGAACATTGAGTTCTTCACCAAGAACTCTGCCTTCCCCAAGACC
ACCAATGGCTGCTCCCAGGCCATGGCTGCCCTGCAGAACCTGCCCCAG
TGCTCCCCTGATGAGATCATGGCCTATGCCCAGAAGATATTCAAGATC
CTGGATGAGGAGCGGGACAAGGTGCTGACCCACATTGACCACATCTTC
ATGGACATCCTGACCACCTGTGTGGAGACCATGTGCAATGAGTACAAG
GTGACCTCTGATGCCTGCATGATGACCATGTATGGCGGCATCTCCCTG
CTGTCTGAGTTCTGCCGGGTGCTGTGCTGCTATGTGCTGGAGGAGACC
TCTGTGATGCTGGCCAAGCGGCCCCTGATCACCAAGCCTGAGGTGATC
TCTGTGATGAAGCGGCGGATTGAGGAGATCAGCATGAAGGTCTTTGCC
CAGTACATCCTGGGCGCTGACCCTCTGCGGGTCTGCTCCCCATCTGTG
GATGACCTGCGGGCCATTGCTGAGGAGTCTGATGAGGAGGAGGCCATT
GTGGCCTACACCCTGGCCACAGCTGGCGTCTCCTCCTCTGACTCCCTG
GTCTCCCCCCCTGAGTCCCCTGTGCCTGCCACCATCCCCCTGTCCTCT
GTGATTGTGGCTGAGAACTCTGACCAGGAGGAGTCTGAGCAGTCTGAT
GAGGAGGAGGAGGAGGGTGCCCAGGAGGAGCGGGAGGACACAGTCTCT
GTGAAGTCTGAGCCTGTCTCTGAGATTGAGGAGGTGGCCCCTGAGGAG
GAGGAGGATGGCGCTGAGGAGCCCACAGCCTCTGGCGGCAAGTCCACC
CATCCCATGGTGACCCGGTCCAAGGCTGACCAG

The amino acid sequence of a modified IE1 protein, designated herein as “mIE1,” is set forth as SEQ ID NO:9:

| (SEQ ID NO: 9) | |
|---|---|
| 1 | MPEKDVLAEL VKQIKVRVDM VRHRIKEHML KKYTQTEEKF TGAFNMMGGC |
| 51 | LQNALDILDK VHEPFEEMKC IGLTMQSMYE NYIVPEDKRE MWMACIKELH |
| 101 | DVSKGAANKL GGALQAKARA KKDELRRKMM YMCYRNIEFF TKNSAFPKTT |
| 151 | NGCSQAMAAL QNLPQCSPDE IMAYAQKIFK ILDEERDKVL THIDHIFMDI |
| 201 | LTTCVETMCN EYKVTSDACM MTMYGGISLL SEFCRVLCCY VLEETSVMLA |
| 251 | KRPLITKPEV ISVMGGGIEE ICMKVFAQYI LGADPLRVCS PSVDDLRAIA |
| 301 | EESDEEEAIV AYTLATAGVS SSDSLVSPPE SPVPATIPLS SVIVAENSDQ |
| 351 | EESEQSDEEE EEGAQEERED TVSVKSEPVS EIEEVAPEEE EDGAEEPTAS |
| 401 | GGKSTHPMVT RSKADQ. |

NLS1 of wild-type IE1 is removed in mIE1 due to a NH₂-terminal truncation from amino acids 2-76 of the wild-type IE1 sequence. This truncation also removes the majority of IE1 encoded by exon 3. mIE1 also has three amino acid substitutions that eliminate function of NLS2: K340G, R341G and R342G of SEQ ID NO:6. Due to the NH₂-terminal truncation, the three mutated amino acid residues are located at residue numbers 265, 266 and 267 of mIE1 (underlined above in SEQ ID NO:9).

The nucleic acid sequence that encodes mIE1, designated here in as “mIE1 (nuc),” is set forth in SEQ ID NO:10:

(SEQ ID NO: 10)
ATGCCTGAGAAGGATGTGCTGGCTGAGCTGGTGAAGCAGATCAAGGTG
CGGGTGGACATGGTGCGGCATCGGATCAAGGAGCACATGCTGAAGAAG
TACACCCAGACAGAGGAGAAGTTCACAGGCGCCTTCAACATGATGGGT
GGCTGCCTGCAGAATGCCCTGGACATCCTGGACAAGGTGCATGAGCCA
TTTGAGGAGATGAAGTGCATTGGCCTGACCATGCAGTCCATGTATGAG
AACTACATTGTGCCTGAGGACAAGCGGGAGATGTGGATGGCCTGCATC
AAGGAGCTGCATGATGTCTCCAAGGGCGCTGCCAACAAGCTGGGCGGT
GCCCTGCAGGCCAAGGCCCGGGCCAAGAAGGATGAGCTGCGGCGGAAG
ATGATGTACATGTGCTACCGGAACATTGAGTTCTTCACCAAGAACTCT
GCCTTCCCCAAGACCACCAATGGCTGCTCCCAGGCCATGGCTGCCCTG
CAGAACCTGCCCCAGTGCTCCCCTGATGAGATCATGGCCTATGCCCAG
AAGATATTCAAGATCCTGGATGAGGAGCGGGACAAGGTGCTGACCCAC
ATTGACCACATCTTCATGGACATCCTGACCACCTGTGTGGAGACCATG
TGCAATGAGTACAAGGTGACCTCTGATGCCTGCATGATGACCATGTAT
GGCGGCATCTCCCTGCTGTCTGAGTTCTGCCGGGTGCTGTGCTGCTAT
GTGCTGGAGGAGACCTCTGTGATGCTGGCCAAGCGGCCCCTGATCACC
AAGCCTGAGGTGATCTCTGTGATGGGTGGCGGTATTGAGGAGATCAGC
ATGAAGGTCTTTGCCCAGTACATCCTGGGCGCTGACCCTCTGCGGGTC
TGCTCCCCATCTGTGGATGACCTGCGGGCCATTGCTGAGGAGTCTGAT
GAGGAGGAGGCCATTGTGGCCTACACCCTGGCCACAGCTGGCGTCTCC
TCCTCTGACTCCCTGGTCTCCCCCCCTGAGTCCCCTGTGCCTGCCACC
ATCCCCCTGTCCTCTGTGATTGTGGCTGAGAACTCTGACCAGGAGGAG
TCTGAGCAGTCTGATGAGGAGGAGGAGGAGGGTGCCCAGGAGGAGCGG
GAGGACACAGTCTCTGTGAAGTCTGAGCCTGTCTCTGAGATTGAGGAG
GTGGCCCCTGAGGAGGAGGAGGATGGCGCTGAGGAGCCCACAGCCTCT
GGCGGCAAGTCCACCCATCCCATGGTGACCCGGTCCAAGGCTGACCAG.

This sequence was constructed synthetically using Lathe codon optimization algorithms (Lathe, 1985, supra).

The wildtype amino acid sequence for human CMV IE2, designated herein as “IE2,” is set forth as SEQ ID NO:11:

| (SEQ ID NO: 11) | |
|---|---|
| 1 | MESSAKRKMD PDNPDEGPSS KVPRPETPVT KATTFLQTML RKEVNSQLSL |
| 51 | GDPLFPELAE ESLKTFEQVT EDCNENPEKD VLAELGDILA QAVNHAGIDS |
| 101 | SSTGPTLTTH SCSVSSAPLN KPTPTSVAVT NTPLPGASAT PELSPRKKPR |
| 151 | KTTRPFKVII KPPVPPAPIM LPLIKQEDIK PEPDFTIQYR NKIIDTAGCI |
| 201 | VISDSEEEQG EEVETRGATA SSPSTGSGTP RVTSPTHPLS QMNHPPLPDP |
| 251 | LGRPDEDSSS SSSSSCSSAS DSESESEEMK CSSGGGASVT SSHHGRGGFG |
| 301 | GAASSSLLSC GHQSSGGAST GPRKKKSKRI SELDNEKVRN IMKDKNTPFC |
| 351 | TPNVQTRRGR VKIDEVSRMF RNTNRSLEYK NLPFTIPSMH QVLDEAIKAC |
| 401 | KTMQVNNKGI QIIYTRNHEV KSEVDAVRCR LGTMCNLALS TPFLMEHTMP |
| 451 | VTHPPEVAQR TADACNEGVK AAWSLKELHT HQLCPRSSDY RNMIIHAATP |
| 501 | VDLLGALNLC LPLMQKFPKQ VMVRIFSTNQ GGFMLPIYET AAKAYAVGQF |
| 551 | EQPTETPPED LDTLSLAIEA AIQDLRNKSQ. |

The two NLSs of IE2 are underlined above: NLS1 (amino acids 145-154) and NLS2 (amino acids 322-329). The portion of IE2 that is encoded by exon 3 spans amino acid 25-85 of SEQ ID NO:11. The two amino acid residues at position 447 and 453, each histidines, are thought to participate in DNA binding activity and are also underlined above. IE2 is encoded by the nucleic acid sequence as set forth in SEQ ID NO:12. These sequences are also represented by NCBI Accession nos. P19893 (protein) and NC_{—}001347.2 (joining nucleotides 170295-171781, 173327-173511, and 173626-173696) (nucleic acid). A codon-optimized nucleic acid sequence that encodes wild-type HCMV IE2, IE2.syn, and was generated using Lathe codon optimization algorithms (Lathe, 1985, supra) is set forth as SEQ ID NO: 13.

(SEQ ID NO: 13)
ATGGAGTCCTCTGCCAAGCGGAAGATGGACCCTGACAACCCTGATGAG
GGCCCATCCTCCAAGGTGCCCCGGCCTGAGACCCCTGTGACCAAGGCC
ACCACCTTCCTGCAGACCATGCTGCGGAAGGAGGTGAACTCCCAGCTG
TCCCTGGGCGACCCCCTGTTCCCTGAGCTGGCTGAGGAGTCCCTGAAG
ACCTTTGAGCAGGTGACAGAGGACTGCAATGAGAACCCTGAGAAGGAT
GTGCTGGCTGAGCTGGGCGACATCCTGGCCCAGGCTGTGAACCATGCT
GGCATTGACTCCTCCTCCACAGGCCCCACCCTGACCACCCACTCCTGC
TCTGTCTCCTCTGCCCCCCTGAACAAGCCCACCCCCACCTCTGTGGCT
GTGACCAACACCCCCCTGCCTGGCGCCTCTGCCACCCCTGAGCTGTCC
CCCCGGAAGAAGCCCCGGAAGACCACCCGGCCATTCAAGGTGATCATC
AAGCCCCCTGTGCCCCCTGCCCCCATCATGCTGCCCCTGATCAAGCAG
GAGGACATCAAGCCTGAGCCTGACTTCACCATCCAGTACCGGAACAAG
ATCATTGACACAGCTGGCTGCATTGTGATCTCTGACTCTGAGGAGGAG
CAGGGCGAGGAGGTGGAGACCCGGGGCGCCACAGCCTCCTCCCCATCC
ACAGGCTCTGGCACCCCCCGGGTGACCTCCCCCACCCATCCCCTGTCC
CAGATGAACCATCCCCCCCTGCCTGACCCCCTGGGCCGGCCTGATGAG
GACTCCTCCTCCTCCTCCTCCTCCTCCTGCTCCTCTGCCTCTGACTCT
GAGTCTGAGTCTGAGGAGATGAAGTGCTCCTCTGGCGGCGGCGCCTCT
GTGACCTCCTCCCATCATGGCCGGGGCGGCTTTGGCGGCGCTGCCTCC
TCCTCCCTGCTGTCCTGTGGCCATCAGTCCTCTGGCGGCGCCTCCACA
GGCCCCCGGAAGAAGAAGTCCAAGCGGATCTCTGAGCTGGACAATGAG
AAGGTGCGGAACATCATGAAGGACAAGAACACCCCATTCTGCACCCCC
AATGTGCAGACCCGGCGGGGCCGGGTGAAGATTGATGAGGTCTCCCGG
ATGTTCCGGAACACCAACCGGTCCCTGGAGTACAAGAACCTGCCATTC
ACCATCCCATCCATGCATCAGGTGCTGGATGAGGCCATCAAGGCCTGC
AAGACCATGCAGGTGAACAACAAGGGCATCCAGATCATCTACACCCGG
AACCATGAGGTGAAGTCTGAGGTGGATGCTGTGCGGTGCCGGCTGGGC
ACCATGTGCAACCTGGCCCTGTCCACCCCATTCCTGATGGAGCACACC
ATGCCTGTGACCCATCCCCCTGAGGTGGCCCAGCGGACAGCTGATGCC
TGCAATGAGGGCGTGAAGGCTGCCTGGTCCCTGAAGGAGCTGCACACC
CATCAGCTGTGCCCCCGGTCCTCTGACTACCGGAACATGATCATCCAT
GCTGCCACCCCTGTGGACCTGCTGGGCGCCCTGAACCTGTGCCTGCCC
CTGATGCAGAAGTTCCCCAAGCAGGTGATGGTGCGGATCTTCTCCACC
AACCAGGGCGGCTTCATGCTGCCCATCTATGAGACAGCTGCCAAGGCC
TATGCTGTGGGCCAGTTTGAGCAGCCCACAGAGACCCCCCCTGAGGAC
CTGGACACCCTGTCCCTGGCCATTGAGGCTGCCATCCAGGACCTGCGG
AACAAGTCCCAG

The amino acid sequence of a modified IE2 protein, designated herein as “IE2(H2A),” is set forth as SEQ ID NO:14:

(SEQ ID NO:14)

| | |
|---|---|
| 1 | MESSAKRKMD PDNPDEGPSS KVPRPETPVT KATTFLQTML |
| | RKEVNSQLSL |
| 51 | GDPLFPELAE ESLKTFEQVT EDCNENPEKD VLAELGDILA |
| | QAVNHAGIDS |
| 101 | SSTGPTLTTH SCSVSSAPLN KPTPTSVAVT NTPLPGASAT |
| | PELSPRKKPR |
| 151 | KTTRPFKVII KPPVPPAPIM LPLIKQEDIK PEPDFTIQYR |
| | NKIIDTAGCI |
| 201 | VISDSEEEQG EEVETRGATA SSPSTGSGTP RVTSPTHPLS |
| | QMNHPPLPDP |
| 251 | LGRPDEDSSS SSSSSCSSAS DSESESEEMK CSSGGGASVT |
| | SSHHGRGGFG |
| 301 | GAASSSLLSC GHQSSGGAST GPRKKKSKRI SELDNEKVRN |
| | IMKDKNTPFC |
| 351 | TPNVQTRRGR VKIDEVSRMF RNTNRSLEYK NLPFTIPSMH |
| | QVLDEAIKAC |
| 401 | KTMQVNNKGI QIIYTRNHEV KSEVDAVRCR LGTMCNLALS |
| | TPFLMEATMP |
| 451 | VTAPPEVAQR TADACNEGVK AAWSLKELHT HQLCPRSSDY |
| | RNMIIHAATP |
| 501 | VDLLGALNLC LPLMQKFPKQ VMVRIFSTNQ GGFMLPIYET |
| | AAKAYAVGQF |
| 551 | EQPTETPPED LDTLSLAIEA AIQDLRNKSQ. |

IE2 (H2A) as two amino acid substitutions (underlined in SEQ ID NO:14) in comparison to the wild-type IE2 protein: H447A and H453A. The mutations were introduced to nullify the ability of IE2 to negatively regulate MIEP activity.

A codon-optimized, nucleic acid sequence that encodes IE2(H2A), designated herein as “IE2(H2A) (nuc),” is set forth in SEQ ID NO:15:

(SEQ ID NO: 15)
ATGGAGTCCTCTGCCAAGCGGAAGATGGACCCTGACAACCCTGATGA
GGGCCCATCCTCCAAGGTGCCCCGGCCTGAGACCCCTGTGACCAAGG
CCACCACCTTCCTGCAGACCATGCTGCGGAAGGAGGTGAACTCCCAG
CTGTCCCTGGGCGACCCCCTGTTCCCTGAGCTGGCTGAGGAGTCCCT
GAAGACCTTTGAGCAGGTGACAGAGGACTGCAATGAGAACCCTGAGA
AGGATGTGCTGGCTGAGCTGGGCGACATCCTGGCCCAGGCTGTGAAC
CATGCTGGCATTGACTCCTCCTCCACAGGCCCCACCCTGACCACCCA
CTCCTGCTCTGTCTCCTCTGCCCCCCTGAACAAGCCCACCCCCACCT
CTGTGGCTGTGACCAACACCCCCCTGCCTGGCGCCTCTGCCACCCCT
GAGCTGTCCCCCCGGAAGAAGCCCCGGAAGACCACCCGGCCATTCAA
GGTGATCATCAAGCCCCCTGTGCCCCCTGCCCCCATCATGCTGCCCC
TGATCAAGCAGGAGGACATCAAGCCTGAGCCTGACTTCACCATCCAG
TACCGGAACAAGATCATTGACACAGCTGGCTGCATTGTGATCTCTGA
CTCTGAGGAGGAGCAGGGCGAGGAGGTGGAGACCCGGGGCGCCACAG
CCTCCTCCCCATCCACAGGCTCTGGCACCCCCCGGGTGACCTCCCCC
ACCCATCCCCTGTCCCAGATGAACCATCCCCCCCTGCCTGACCCCCT
GGGCCGGCCTGATGAGGACTCCTCCTCCTCCTCCTCCTCCTCCTGCT
CCTCTGCCTCTGACTCTGAGTCTGAGTCTGAGGAGATGAAGTGCTCC
TCTGGCGGCGGCGCCTCTGTGACCTCCTCCCATCATGGCCGGGGCGG
CTTTGGCGGCGCTGCCTCCTCCTCCCTGCTGTCCTGTGGCCATCAGT
CCTCTGGCGGCGCCTCCACAGGCCCCCGGAAGAAGAAGTCCAAGCGG
ATCTCTGAGCTGGACAATGAGAAGGTGCGGAACATCATGAAGGACAA
GAACACCCCATTCTGCACCCCCAATGTGCAGACCCGGCGGGGCCGGG
TGAAGATTGATGAGGTCTCCCGGATGTTCCGGAACACCAACCGGTCC
CTGGAGTACAAGAACCTGCCATTCACCATCCCATCCATGCATCAGGT
GCTGGATGAGGCCATCAAGGCCTGCAAGACCATGCAGGTGAACAACA
AGGGCATCCAGATCATCTACACCCGGAACCATGAGGTGAAGTCTGAG
GTGGATGCTGTGCGGTGCCGGCTGGGCACCATGTGCAACCTGGCCCT
GTCCACCCCATTCCTGATGGAGGCCACCATGCCTGTGACAGCCCCCC
CTGAGGTGGCCCAGCGGACAGCTGATGCCTGCAATGAGGGCGTGAAG
GCTGCCTGGTCCCTGAAGGAGCTGCACACCCATCAGCTGTGCCCCCG
GTCCTCTGACTACCGGAACATGATCATCCATGCTGCCACCCCTGTGG
ACCTGCTGGGCGCCCTGAACCTGTGCCTGCCCCTGATGCAGAAGTTC
CCCAAGCAGGTGATGGTGCGGATCTTCTCCACCAACCAGGGCGGCTT
CATGCTGCCCATCTATGAGACAGCTGCCAAGGCCTATGCTGTGGGCC
AGTTTGAGCAGCCCACAGAGACCCCCCCTGAGGACCTGGACACCCTG
TCCCTGGCCATTGAGGCTGCCATCCAGGACCTGCGGAACAAGTCCCA
G.

The codon-optimization of this sequence was generated using Lathe codon optimization algorithms (Lathe, 1985, supra).

The amino acid sequence of a modified IE2 protein, designated herein as “mIE2,” is set forth as SEQ ID NO:16:

(SEQ ID NO: 16)

| | |
|---|---|
| 1 | MGDILAQAVN HAGIDSSSTG PTLTTHSCSV SSAPLNKPTP |
| | TSVAVTNTPL |
| 51 | PGASATPELS PSSGPRKTTR PFKVIIKPPV PPAPIMLPLI |
| | KQEDIKPEPD |
| 101 | FTIQYRNKII DTAGCIVISD SEEEQGEEVE TRGATASSPS |
| | TGSGTPRVTS |
| 151 | PTHPLSQMNH PPLPDPLGRP DEDSSSSSSS SCSSASDSES |
| | ESEEMKCSSG |
| 201 | GGASVTSSHH GRGGFGGAAS SSLLSCGHQS SGGASTGPRS |
| | SGSKRISELD |
| 251 | NEKVRNIMKD KNTPFCTPNV QTRRGRVKID EVSRMFRNTN |
| | RSLEYKNLPF |
| 301 | TIPSMHQVLD EAIKACKTMQ VNNKGIQIIY TRNHEVKSEV |
| | DAVRCRLGTM |
| 351 | CNLALSTPFL MEHTMPVTHP PEVAQRTADA CNEGVKAAWS |
| | LKELHTHQLC |
| 401 | PRSSDYRNMI IHAATPVDLL GALNLCLPLM QKFPKQVMVR |
| | IFSTNQGGFM |
| 451 | LPIYETAAKA YAVGQFEQPT ETPPEDLDTL SLAIEAAIQD |
| | LRNKSQ. |

mIE2 has three amino acid substitutions in comparison to the wild-type sequence that eliminates the function of NLS1: R146S, K147S and K148G of SEQ ID NO:11. Due to an NH₂-terminal truncation, these three mutated amino acid residues are located at positions 62, 63 and 64 of mIE2 (underlined in SEQ ID NO:16). mIE2 also has three amino acid substitutions in comparison to the wild-type sequence to eliminate function of NLS2: K324S, K325S and K326G of SEQ ID NO:11. Again, due to an NH₂-terminal truncation, these mutated amino acid residues are located at positions 240, 241 and 242 (underlined in SEQ ID NO:16). mIE2 also has an NH₂-terminal truncation corresponding to amino acids 2-85 of the wild-type IE2 sequence that removes an additional, putative NLS within exon 2, as well as the majority of the amino acid sequence encoded by exon 3.

A codon-optimized, nucleic acid sequence that encodes mIE2, designated herein as “mIE2 (nuc),” is set forth in SEQ ID NO:17:

(SEQ ID NO: 17)
ATGGGCGACATCCTGGCCCAGGCTGTGAACCATGCTGGCATTGACT
CCTCCTCCACAGGCCCCACCCTGACCACCCACTCCTGCTCTGTCTC
CTCTGCCCCCCTGAACAAGCCCACCCCCACCTCTGTGGCTGTGACC
AACACCCCCCTGCCTGGCGCCTCTGCCACCCCTGAGCTGTCCCCCT
CTTCTGGTCCCCGGAAGACCACCCGGCCATTCAAGGTGATCATCAA
GCCCCCTGTGCCCCCTGCCCCCATCATGCTGCCCCTGATCAAGCAG
GAGGACATCAAGCCTGAGCCTGACTTCACCATCCAGTACCGGAACA
AGATCATTGACACAGCTGGCTGCATTGTGATCTCTGACTCTGAGGA
GGAGCAGGGCGAGGAGGTGGAGACCCGGGGCGCCACAGCCTCCTCC
CCATCCACAGGCTCTGGCACCCCCCGGGTGACCTCCCCCACCCATC
CCCTGTCCCAGATGAACCATCCCCCCCTGCCTGACCCCCTGGGCCG
GCCTGATGAGGACTCCTCCTCCTCCTCCTCCTCCTCCTGCTCCTCT
GCCTCTGACTCTGAGTCTGAGTCTGAGGAGATGAAGTGCTCCTCTG
GCGGCGGCGCCTCTGTGACCTCCTCCCATCATGGCCGGGGCGGCTT
TGGCGGCGCTGCCTCCTCCTCCCTGCTGTCCTGTGGCCATCAGTCC
TCTGGCGGCGCCTCCACAGGCCCCCGGTCTTCTGGTTCCAAGCGGA
TCTCTGAGCTGGACAATGAGAAGGTGCGGAACATCATGAAGGACAA
GAACACCCCATTCTGCACCCCCAATGTGCAGACCCGGCGGGGCCGG
GTGAAGATTGATGAGGTCTCCCGGATGTTCCGGAACACCAACCGGT
CCCTGGAGTACAAGAACCTGCCATTCACCATCCCATCCATGCATCA
GGTGCTGGATGAGGCCATCAAGGCCTGCAAGACCATGCAGGTGAAC
AACAAGGGCATCCAGATCATCTACACCCGGAACCATGAGGTGAAGT
CTGAGGTGGATGCTGTGCGGTGCCGGCTGGGCACCATGTGCAACCT
GGCCCTGTCCACCCCATTCCTGATGGAGCACACCATGCCTGTGACC
CATCCCCCTGAGGTGGCCCAGCGGACAGCTGATGCCTGCAATGAGG
GCGTGAAGGCTGCCTGGTCCCTGAAGGAGCTGCACACCCATCAGCT
GTGCCCCCGGTCCTCTGACTACCGGAACATGATCATCCATGCTGCC
ACCCCTGTGGACCTGCTGGGCGCCCTGAACCTGTGCCTGCCCCTGA
TGCAGAAGTTCCCCAAGCAGGTGATGGTGCGGATCTTCTCCACCAA
CCAGGGCGGCTTCATGCTGCCCATCTATGAGACAGCTGCCAAGGCC
TATGCTGTGGGCCAGTTTGAGCAGCCCACAGAGACCCCCCCTGAGG
ACCTGGACACCCTGTCCCTGGCCATTGAGGCTGCCATCCAGGACCT
GCGGAACAAGTCCCAG.

The codon-optimization of this sequence was generated using Lathe codon optimization algorithms (Lathe, 1985, supra).

The amino acid sequence of a modified IE2 protein, designated herein as “mIE2(H2A),” is set forth as SEQ ID NO:18:

(SEQ ID NO: 18)

| | |
|---|---|
| 1 | MGDILAQAVN HAGIDSSSTG PTLTTHSCSV SSAPLNKPTP |
| | TSVAVTNTPL |
| 51 | PGASATPELS PSSGPRKTTR PFKVIIKPPV PPAPIMLPLI |
| | KQEDIKPEPD |
| 101 | FTIQYRNKII DTAGCIVISD SEEEQGEEVE TRGATASSPS |
| | TGSGTPRVTS |
| 151 | PTHPLSQMNH PPLPDPLGRP DEDSSSSSSS SCSSASDSES |
| | ESEEMKCSSG |
| 201 | GGASVTSSHH GRGGFGGAAS SSLLSCGHQS SGGASTGPRS |
| | SGSKRISELD |
| 251 | NEKVRNIMKD KNTPFCTPNV QTRRGRVKID EVSRMFRNTN |
| | RSLEYKNLPF |
| 301 | TIPSMHQVLD EAIKACKTMQ VNNKGIQIIY TRNHEVKSEV |
| | DAVRCRLGTM |
| 351 | CNLALSTPFL MEATMPVTAP PEVAQRTADA CNEGVKAAWS |
| | LKELHTHQLC |
| 401 | PRSSDYRNMI IHAATPVDLL GALNLCLPLM QKFPKQVMVR |
| | IFSTNQGGFM |
| 451 | LPIYETAAKA YAVGQFEQPT ETPPEDLDTL SLAIEAAIQD |
| | LRNKSQ. |

mIE2(H2A) has a combination of the mutations present in IE2(H2A) and mIE2. There are two amino acid substitutions to nullify the ability of the protein to negatively regulate MIEP activity. These mutations are located at H363A and H369A of SEQ ID NO:18, corresponding to H447A and H453A of the wild-type IE2 amino acid sequence. mIE2(H2A) has an NH₂-terminal truncation corresponding to amino acids 2-85 of the wild-type IE2 sequence that removes a putative NLS within exon 1, as well as the majority of the amino acid sequence encoded by exon 3. There are also three amino acid substitutions in comparison to the wild-type IE2 sequence that eliminate function of NLS1: R146S, K147S and K148G of SEQ ID NO:11. These three mutated amino acid residues are located at positions 62, 63 and 64 of mIE2 (underlined in SEQ ID NO:18). There are also three amino acid substitutions in comparison to the wild-type sequence to eliminate function of NLS2: K324S, K325S and K326G of SEQ ID NO:11. Due to the NH₂-terminal truncation, these mutated amino acid residues are located at positions 240, 241 and 242 (underlined in SEQ ID NO:18).

A codon-optimized, nucleic acid sequence that encodes mIE2(H2A), designated herein as “mIE2(H2A) (nuc),” is set forth in SEQ ID NO:19:

(SEQ ID NO: 19)
ATGGGCGACATCCTGGCCCAGGCTGTGAACCATGCTGGCATTGACT
CCTCCTCCACAGGCCCCACCCTGACCACCCACTCCTGCTCTGTCTC
CTCTGCCCCCCTGAACAAGCCCACCCCCACCTCTGTGGCTGTGACC
AACACCCCCCTGCCTGGCGCCTCTGCCACCCCTGAGCTGTCCCCCT
CTTCTGGTCCCCGGAAGACCACCCGGCCATTCAAGGTGATCATCAA
GCCCCCTGTGCCCCCTGCCCCCATCATGCTGCCCCTGATCAAGCAG
GAGGACATCAAGCCTGAGCCTGACTTCACCATCCAGTACCGGAACA
AGATCATTGACACAGCTGGCTGCATTGTGATCTCTGACTCTGAGGA
GGAGCAGGGCGAGGAGGTGGAGACCCGGGGCGCCACAGCCTCCTCC
CCATCCACAGGCTCTGGCACCCCCCGGGTGACCTCCCCCACCCATC
CCCTGTCCCAGATGAACCATCCCCCCCTGCCTGACCCCCTGGGCCG
GCCTGATGAGGACTCCTCCTCCTCCTCCTCCTCCTCCTGCTCCTCT
GCCTCTGACTCTGAGTCTGAGTCTGAGGAGATGAAGTGCTCCTCTG
GCGGCGGCGCCTCTGTGACCTCCTCCCATCATGGCCGGGGCGGCTT
TGGCGGCGCTGCCTCCTCCTCCCTGCTGTCCTGTGGCCATCAGTCC
TCTGGCGGCGCCTCCACAGGCCCCCGGTCTTCTGGTTCCAAGCGGA
TCTCTGAGCTGGACAATGAGAAGGTGOGGAACATCATGAAGGACAA
GAACACCCCATTCTGCACCCCCAATGTGCAGACCCGGCGGGGCCGG
GTGAAGATTGATGAGGTCTCCCGGATGTTCCGGAACACCAACCGGT
CCCTGGAGTACAAGAACCTGCCATTCACCATCCCATCCATGCATCA
GGTGCTGGATGAGGCCATCAAGGCCTGCAAGACCATGCAGGTGAAC
AACAAGGGCATCCAGATCATCTACACCCGGAACCATGAGGTGAAGT
CTGAGGTGGATGCTGTGCGGTGCCGGCTGGGCACCATGTGCAACCT
GGCCCTGTCCACCCCATTCCTGATGGAGGCCACCATGCCTGTGACA
GCCCCCCCTGAGGTGGCCCAGCGGACAGCTGATGCCTGCAATGAGG
GCGTGAAGGCTGCCTGGTCCCTGAAGGAGCTGCACACCCATCAGCT
GTGCCCCCGGTCCTCTGACTACCGGAACATGATCATCCATGCTGCC
ACCCCTGTGGACCTGCTGGGCGCCCTGAACCTGTGCCTGCCCCTGA
TGCAGAAGTTCCCCAAGCAGGTGATGGTGCGGATCTTCTCCACCAA
CCAGGGCGGCTTCATGCTGCCCATCTATGAGACAGCTGCCAAGGCC
TATGCTGTGGGCCAGTTTGAGCAGCCCACAGAGACCCCCCCTGAGG
ACCTGGACACCCTGTCCCTGGCCATTGAGGCTGCCATCCAGGACCT
GCGGAACAAGTCCCAG.

The codon-optimization of this sequence was generated using Lathe codon optimization algorithms (Lathe, 1985, supra).

### Example 3

### Expression of Inactivated pp65, IE1 and IE2

Plasmid vector construction—DNA sequence corresponding to pp65 open reading frame (ORF) was PCR amplified from AD169 viral genome DNA. The fragment was cloned into pV1Jns vector (SEQ ID NO:28), as described in J. Shiver et. al. in DNA Vaccines, M. Liu et al. eds., N.Y. Acad. Sci., N.Y., 772:198-208 (1996), and authenticity of the fragment was confirmed by restriction digestion and DNA sequencing. The mpp65 ORF and full-length, codon optimized wild type IE1 and IE2 genes were synthetically generated. Mutagenesis primers were designed for deletions or substitution mutations for IE1- and IE2-related constructs and used in sewing PCR method using high fidelity polymerase (Stratagene). Fragments were purified through electrophoresis on 1% agarose gel and cloned into pV1Jns expression vector using In-Fusion cloning kit (Clontech). The constructs were confirmed by restriction enzyme digestion and DNA sequencing.

Adenoviral vector construction—The methods for construction and characterization of Ad vectors have been published (Curiel, D. T & Douglas, J. T. (Eds.). (2002). Adenoviral Vectors for Gene Therapy. San Diego: Academic Press). Briefly, the selected DNA constructs were cloned into psNEBAd6 shuttle vector using In-Fusion cloning kit (Clontech), and the inserts were confirmed through restriction digest and DNA sequencing. The confirmed shuttle vectors underwent homologous recombination with pMRKAd6DE1 (ΔE1) or pMRKAd6DE1DE3 (ΔE1ΔE3) (see Emini et al., US20040247615) in E. coli BJ5183 cells. The pre-Ad6 plasmid was verified by a Hind III restriction enzyme analysis, and transfected into PerC.6 cells. The supernatant was harvested when confirmed CPE, and the virus was passaged in PerC.6 cells.

Western blot analysis—Cell lysates were prepared from HEK293 cells transfected with 2 μg of pV1Jns containing CMV antigens using GeneJammer (Stratagene) transfection reagent or Per.C6 cells infected with Adenovirus vectors. The cell lysates were denatured and separated on a 4-20% SDS-PAGE (Novex). The proteins were transferred to nitrocellulose membrane (Invitrogen) and blotted with mouse mAb specific to CMV antigens. For pp65, a mouse mAb was purchased from US Biologicals (Swampscott, Mass.). For IE1 and IE2, two mAbs were purchased from Vancouver LTD which specifically recognize exon 4 (IE1) and exon 5(IE2), respectively. The blot was developed using the WesternBreeze Chromogenic Kit (Invitrogen).

Results—Plasmid-based and/or adenoviral based expression vectors were generated, expressing either wild-type HCMV pp65, IE1 or IE2 proteins or their modified derivatives described in Example 2. A summary of the CMV antigen constructs that were generated are listed in Table 5.

TABLE 5
Summary of CMV antigen constructs

| Antigen | Size | Modification | | | |
|---|---|---|---|---|---|
| (ID) | (amino acids) | (mutation & deletion) | DNA vector | Ad5 vector | Ad6 vector |
| pp65 | 561 | — | — | Ad5-pp65 | Ad6-pp65 |
| mpp65 | 535 | Δ 2 NLS, K436G | — | — | Ad6-mpp65 |
| mpp65 | 535 | Δ 2 NLS, K436G | — | Ad5-mpp65.syn | Ad6-mpp65.syn |
| (mpp65.syn | | | | | |
| nuc. seq.) | | | | | |
| IE1 | 491 | — | V1Jns-IE1 | — | Ad6-IE1 |
| mIE1 | 416 | Δ 2 NLS, | V1Jns-mIE1 | — | Ad6-mIE1 |
| | | Δ exon 2 & 3 | | | |
| IE2 | 580 | — | V1Jns-IE2 | — | — |
| IE2(H2A) | 580 | H447A, H453A | V1Jns-IE2(H2A) | — | Ad6-IE2(H2A) |
| mIE2 | 496 | Δ exon 2 & 3, | V1Jns-mIE2 | — | Ad6-mIE2 |
| | | Δ 2 NLS | | | |
| mIE2(H2A) | 496 | H447A, H453A, | V1Jns-mIE2(H2A) | — | — |
| | | Δ exon 2 &3, | | | |
| | | Δ 2 NLS | | | |

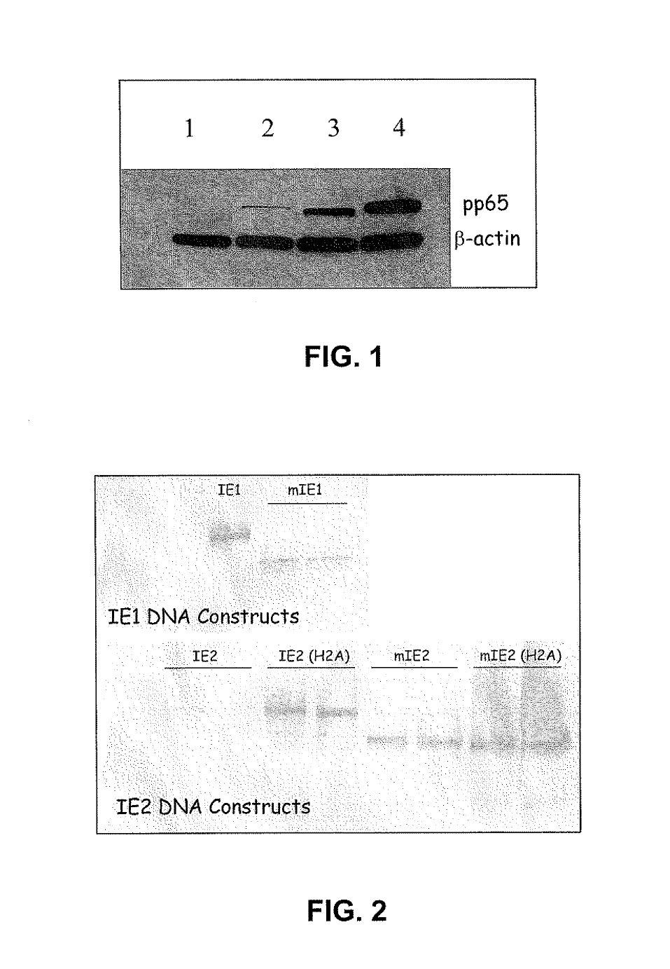

The expression of pp65 and mpp65 from three adenovirus constructs (Ad6-pp65, Ad6-mpp65, and Ad5-pp65) in transfected Per.C6 cells was confirmed by Western blot using a monoclonal antibody to pp65 (see FIG. 1). In FIG. 1, lane 1 is a lysate from Per.C6 cells that have been mock transfected; lane 2 is a lysate from Per.C6 cells transfected with Ad6-pp65; lane 3 is a lysate from Per.C6 cells transfected with Ad6-mpp65; and, lane 4 is a lysate from Per.C6 cells transfected with Ad5-pp65. These constructs were expanded and evaluated in mice for immunogenicity (see Example 4, infra).

Expression of the IE1- and IE2-related DNA constructs (V1Jns-IE1 and V1Jns-IE2) was confirmed in transiently transfected HEK293 cells (FIG. 2). All constructs were evaluated in duplicate cultures to ensure the transfection efficiency. Differential expression levels of wild-type IE1 (“IE1”) versus modified IE1 (“mIE1”) are noted, confirming the ability of the IE1 protein to augment the MIEP activity within the V1Jns vector (Mocarski, Fields Virology, 1996, supra). This ability to enhance MIEP activity was abrogated by the modifications introduced to the mIE1 protein that result in restricting the protein from trafficking to the nucleus. This is noted by the reduced mIE1 expression in comparison to wild-type IE1 expression as shown in FIG. 2. For the IE2-related constructs, differential expression levels between wild-type IE2 (IE2) and its modified forms are also seen. Expression of wild-type IE2 is limited, confirming reports that IE2 down-regulates MIEP activity (Mocarski, Fields Virology, 1996, supra; Petrik et al, 2006, supra). Expression is restored in each of the various modified IE2 constructs. These data suggest that removing the nuclear localization sequences effectively abrogates the protein's negative regulatory function on MIEP.

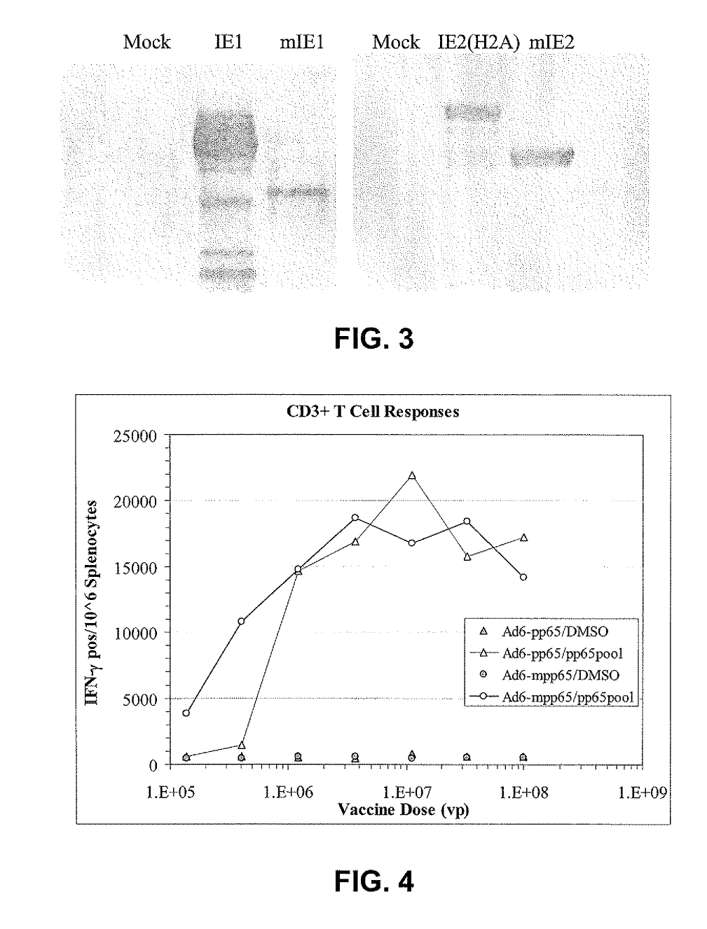

Based on the IE1 and IE2 plasmid expression results, IE1- and IE2-related Ad6 vectors were constructed, e.g., Ad6-IE1, Ad6-mIE1, Ad6-IE2(H2A) and Ad6-mIE2. 1E2(H2A) was selected in place of wild-type IE2 for construction of Ad6 vector to minimize the down regulation of wtIE2 on CMV promoter in Ad6 vector. FIG. 3 shows expression levels for the Ad6 constructs in transfected Per.C6 cells, comparing IE1 versus mIE1 expression and IE2(H2A) versus mIE2 expression. As shown in FIG. 3, there is no enhancement of mIE1 expression (in comparison to IE1 expression) as a result of the restriction of the modified protein from the nucleus. FIG. 3 also confirms the plasmid vector expression data for IE2, showing that a modified IE2 protein (mIE2) that does not contain histidine mutations at position 447 and 453 does not impact protein expression.

### Example 4

### Immunogenicity Analysis in Mice

Vaccination protocol—4-10 weeks old female C57Bl/6×Balb/c F1 mice were immunized with Ad6 constructs i.m. (intramuscular) at week 0. The vaccines were administrated in 100 μL volume with 50 μL injected in each quadriceps. Spleens were harvested from 3-4 animals per group at the indicated time points, and splenocytes were isolated and pooled for immune assays (intracellular cytokine staining or ELISPOT). Serum samples were collected from all animals via tail veins.

Flow cytometry—Mouse splenocytes were isolated and resuspended in R10 medium at 2×10⁷ cells/ml, and 100 μl of cells per well were plated in 96-well U-bottom plates (Corning). Cells were incubated with 100 μl of CMV peptide pools at 3 μg/ml or DMSO mock control in the presence of Brefeldin A (Sigma #B-7651) at 10 μg/ml. The cultures were incubated at 37° C. overnight, and cells were washed once with 2% FBS/PBS. The cells were stained with a cocktail of FITC-conjugated rat anti-mouse CD3 antibody, clone 17A2 (BD Bioscience) and PE-Cy5 conjugated rat anti-mouse CD8α, clone 53.6.7 (BD Bioscience), at room temperature for 20 min in dark. After wash once with 2% FBS/PBS, the cells were permeabilized with Cytofix/Cytoperrn Plus buffer (BD PharMingen) at 4° C. in dark for 20 min. The cells were then stained with 0.1 μg of APC-conjugated rat anti-mouse IFN-γ antibody, clone XMG1.2 (BD Biosience), at 4° C. for 30 min. After wash, the cells were analyzed by fluorescence flow-cytometry on FACS Calibur (Becton Dickinson). Data were analyzed using CellQuest software (Becton Dickinson). Lymphocyte populations were gated based on their forward/side scatter profiles. CD3⁺CD8⁺ cells among lymphocytes were then gated, and the percentage of IFN-γ⁺ cells in this gated population was reported.

ELISPOT assay—Mouse splenocytes were resuspended in R10 medium at 1×10⁷ cells/ml, and seeded in 50 μl (5×10⁵ cells/well) per well onto 96-well MultiScreen-IP white filtration plates (Millipore) coated with 100 μl/well of rat anti-mouse IFN-γ antibody, clone AN18 (MABTECH) at 10 μg/ml in PBS. CMV peptide pools were diluted in R10 to 6 μg/ml per peptide and 50 μl was added to the wells. Negative control wells were added with equal volume R10 containing peptide-free DMSO diluent matching the DMSO concentration in the peptide solution. Plates were incubated at 37° C., 5% CO₂, for 20-24 hrs, and then washed 6 times with 200 μl/well of wash buffer (PBS/0.05% Tween 20). Biotinylated rat anti-mouse IFN-γ antibody, clone R4-6A2 (MABTECH) was added at 100 μl/well at 0.25 μg/ml in PBS/1% FBS. Plates were incubated at 4° C. overnight, and then washed 4 times. Streptavidin-AP (BD PharMingen) was added at 100 μl/well at a 1:3000 dilution and the plate was incubated at room temperature for 60 min before being developed as outlined above.

ELISA assay—Mouse serum samples were collected at week 3 post vaccination. NUNC Maxisorb™ 96-well plates were coated with 50 μl per well of antigen (cell lysate of MRC-5 cells infected with HCMV) at 1:300 dilution in PBS at 4° C. over night. Plates were washed with PBS and blocked with 3% milk in PBS containing 0.05% Tween-20 (milk-PBST). Testing samples were serial diluted in PBST, and the plates were incubated at room temperature for 2 hr. Fifty microliters of diluted HRP-conjugated secondary antibodies in milk-PBST was added per well, and the plates were incubated at room temperature for 1 hr. One hundred microliters of one component TMB substrate (Virolabs, Chantilly, Va.) was added per well. After 5 to 10 min incubation at room temperature in the dark, the reaction was stopped by adding 100 μl of 1N H₂SO₄ per well. The antibody titer is defined as the reciprocal of the highest dilution that yields an OD 450 nm value above 2 times of mean of negative control wells.

Results—Immunogenicities of the HCMV pp65-, IE1- and IE2-related Ad6 constructs were evaluated in C57Bl/6×Balb/c F1 mice. Vaccination dose titration was conducted to demonstrate comparability in immunogenicity of the wild-type antigens versus the modified forms.

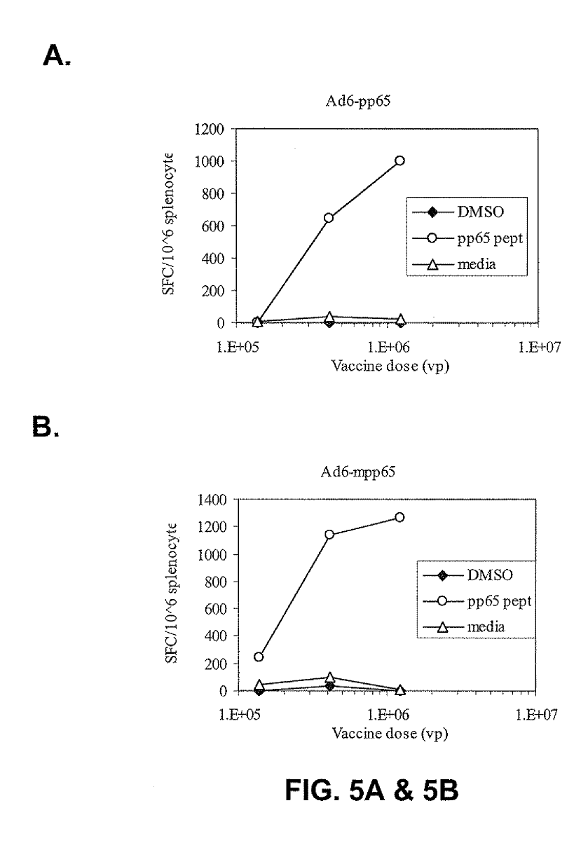

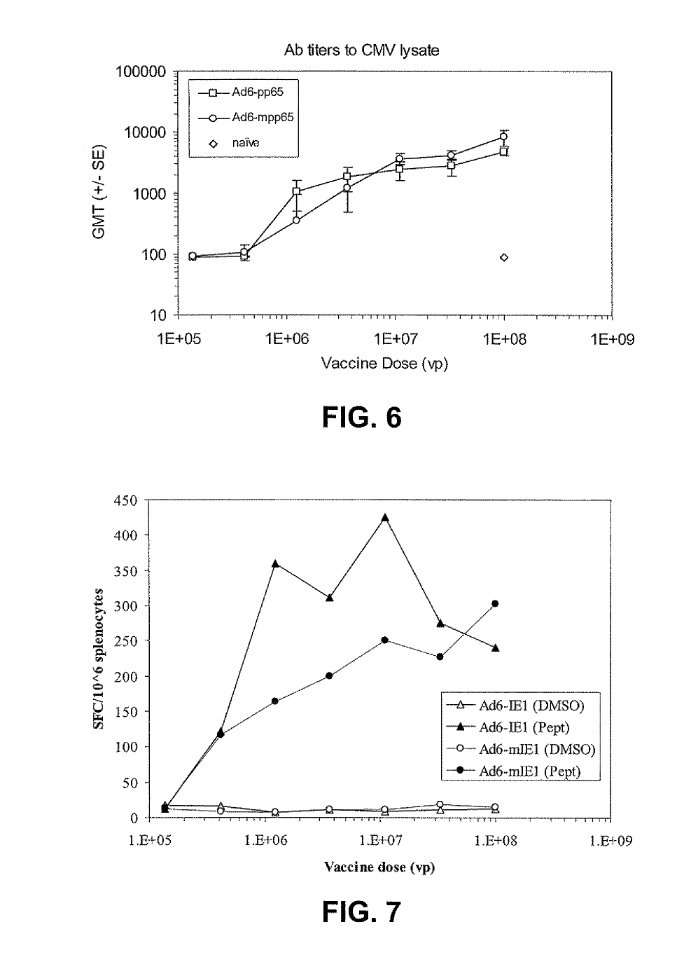

Mice were immunized intramuscularly with Ad6 vectors expressing either wild-type pp65 or modified pp65 (“mpp65”) at viral particle (vp) doses of between 10⁵ to 10⁸. Spleens from three mice were harvested four (4) weeks post vaccination and pooled. The splenocytes were stimulated with either DMSO control or a pp65 peptide pool of 15-mers overlapping by 11 amino acids. IFN-γ producing T cells were measured by flow cytometry, as described (see FIG. 4). ELISPOT assays on selected groups shown in FIG. 4 were performed (FIGS. 5A and 5B), as well as ELISA analysis of sera collected at three (3) weeks post immunization against CMV-infected MRC-5 cell lysate, which contained large amount of pp65 antigen (FIG. 6). The results showed that modification of pp65 antigen (mpp65 construct) did not compromise its immunogenicity in mice, as both Ad6 constructs elicited comparable levels of cellular immune responses and antibody titers to pp65 antigen.

Similarly, mice were immunized intramuscularly with Ad6 vectors expressing IE1 or mIE1 at viral particle (vp) doses of between 10⁵ to 10⁸. Four weeks post immunization, spleens from 4 mice were pooled and evaluated in ELISPOT assays with either DMSO control or an IE1 peptide pool of 15-mers overlapping by 11 amino acids (see FIG. 7). Dose titration responses demonstrated that both Ad6 constructs were immunogenic in mice and elicited comparable levels of ELISPOT responses when stimulated with the IE1 peptide pool. Thus, modifications of IE1 outlined in Table 5 did not compromise its immunogenicity in mice.

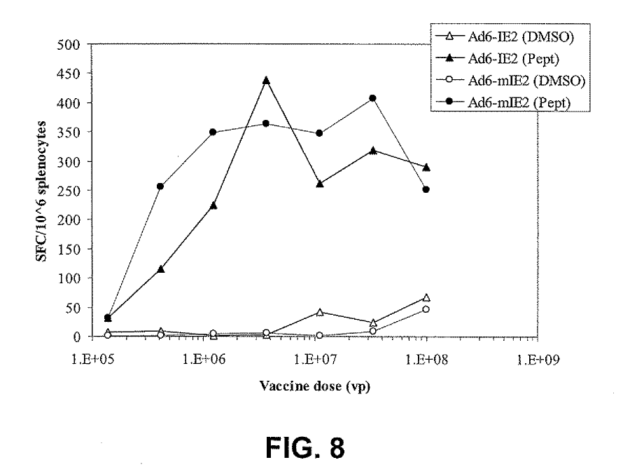

Ad6 vectors expressing full length IE2 with two His-to-Ala substitutions or modified IE2 with exons 2 and 3 deletion and NLS deletion (Table 5) were evaluated in mice in a dose ranging experiment (viral particle (vp) doses of between 10⁵ to 10⁸). Four weeks post immunization, spleens from 4 mice were pooled and evaluated in ELISPOT assays with either DMSO control or an IE2 peptide pool of 15-mers overlapping by 11 amino acids (see FIG. 8). The results confirmed that both Ad6 vectors were immunogenic in mice and can elicit IE2-specific ELISPOT responses. The dose titration curves shown in FIG. 9 indicated that modifications of IE2 (Table 5) had minimal effect on its immunogenicity in mice.

### Example 5

### Subcellular Localization of CMV Antigens

Immunofluorescence protocol—MRC-5 cells were plated in 4-well Lab-Tek II Chamber Slide (Nalgen Nunc International, Naperville, Ill.) at 1×10⁴ cells/well in DMEM medium containing 10% FBS and incubated at 37° C., 5% CO₂, for 48 hr. Cells were infected with Ad6-pp65, Ad6-mpp65, Ad6-IE1, Ad6-mIE1, Ad6-IE2 or Ad6-mIE2 at particle-to-cell ratios of 1000 overnight. Control wells were infected with empty Ad6 vector. Cells were washed once with PBS and fixed with 2% paraformaldehyde in PBS at room temperature for 30 min. Slides were washed twice with PBS buffer containing glycine at 1 mg/ml and once with PBS, and the cells were permeabilized by incubating with 0.2% Triton X-100/0.2% BSA at room temperature for 10 min. Antibodies used for staining were as follows: mouse anti-human CMV IE1 mAb, clone L-14 (ATCC) at 1 μg/ml; rabbit anti-human CMV IE2 immune serum (Merck) at 1:500 dilution; mouse anti-CMV pp65 Tegument Protein (UL83) antibody (US Biological) at 1:50 dilution; rabbit anti-human Sp100 (ND10) polyclonal antibody (Chemicon) at 1:100 dilution; Alexa Fluor 594 chicken anti-rabbit IgG (Invitrogen) at 1:1000 dilution; and Alexa Fluor 488 chicken anti-mouse IgG (Invitrogen) at 1:1000 dilution. All antibodies were diluted in 0.1% Triton X-100/0.2% BSA/PBS solution. Cells were stained with primary antibodies at room temperature for 60 min, washed three times for 5 min each in 0.1% Triton X-100/0.2% BSA/PBS solution, and then incubated with secondary antibodies at room temperature for 60 min. Cells were washed three times with 0.1% Triton X-100/0.2% BSA/PBS solution and once with PBS. Chambers were removed and slides dried briefly in room air. One drop of Vectashield Mounting Medium with DAPI (for nuclear staining) was applied onto each slide, which was then covered with coverslip and sealed with Nail Polish. Images of the cells were taken with a confocal microscope (Nikon Eclipse TE2000-U with the PerkinElmer Ultraview ERS Rapid Confocal Imager system). The scanning procedure itself illuminates the specimen through a Nipkow spinning disc with specific laser emissions at the following wavelengths: 405 nm, 488 nm, 568 nm, and 640 nm.

Results—To examine the effect of the modifications described in Example 2 on HCMV antigens pp65, IE1 and IE2 on their subcellular localization, immunofluorescent staining of MRC-5 cells transfected with various Ad6 constructs was conducted. The fluorescently-stained slides were examined using confocal microscopy. The ND-10 protein, Sp-100, was also imaged to evaluate effects of IE1 on dispersing the ND-10 structure (Maul et al., 2002, J. Struct. Biol. 129:278-287; Castillo and Kowalik, 2002, Gene 290:19-34).

In these studies, wild-type pp65 was predominantly localized to the nucleus; while mpp65 was more evenly distributed between the cytoplasm and the nucleus. This confirms that the modifications in mpp65 by eliminating the bipartite NLS sequence changed the cellular distribution of pp65 from exclusively nuclear to both nuclear and cytoplasmic. It is implicated that additional NLSs exist in pp65 (Schmolke et al, 1995, supra). As expected, the modifications in mpp65 did not affect the localization of ND-10 protein, Sp100, appearing as punctuate staining within the nucleus in both Ad6-pp65- and Ad-mpp65-transfected cells.

Wild-type IE1 was also predominantly localized to the nucleus of the transfected MRC-5 cells. In comparison, there was no nuclear or cytoplasmic staining of mIE1, indicating that the modifications in mIE1 altered or deleted the epitope recognized by the anti-IE1 antibody used for immunofluorescent studies. However, the punctuate, nuclear Sp100 staining was visibly different between cells transfected with Ad6-IE1 and those transfected with Ad6-mIE1. Sp100 staining in cells transfected with Ad6-IE1 was diffuse within the nucleus, confirming the ability of IE1 to disperse the ND-10 structure. However, Sp100 staining in Ad6-mIE1-transfected cells was punctuate, indicating that the modifications in mIE1 alter the protein such that it can no longer disperse ND-10.

Wild-type IE2 is also predominantly localized to the cell nucleus. This nuclear staining is abolished in cells expressing mIE2.

In summary, expression of the Ad6-CMV antigen constructs was confirmed by immunofluorescense staining for all the CMV antigens, except mIE1. Removal of the pp65 nuclear localization signals shifted the protein's subcellular location from exclusively nuclear to both nuclear and cytoplasmic, as reported in literature (Schmolke et al, 1995, supra). Removal of the IE1 NLSs abrogated the protein's ability to disperse ND-10. Removal of the IE2 NLSs changed its location to the cytoplasm. The results of confocal microscopic studies are summarized in Table 6.

TABLE 6
Summary of confocal microscopy studies

| | | | ND-10 |
|---|---|---|---|
| Ad-6 construct | Expression detected | Cellular localization | disruption |
| IE1 | Yes | Nuclear | Yes |
| mIE1 | No | | No |
| IE2(H2A) | Yes | Nuclear | ND |
| mIE2 | Yes | Cytoplasmic | ND |
| pp65 | Yes | Nuclear | No |
| mpp65 | Yes | Both nuclear and | No |
| | | cytoplasmic | |

ND: not determined

### Example 6

### Construction of CMV Fusion Antigens

Fusion constructs of three of the modified CMV antigens described in Example 2 were generated for insertion into an expression vector, e.g., V1Jns DNA plasmid, suitable for DNA vaccination in a mammal. Each transcript is approximately 4.5 Kb in size. Four fusion constructs were generated, designated as “P12,” “P21,” “2P1” and “21P” to represent different antigen fusion orders (see Table 7). Each nucleic acid sequence encoding the modified antigens is codon optimized and was synthetically generated. To reduce the probability of generating undesired and potentially auto-immunogenic T-cell epitopes due to the direct fusion of two open reading frames (ORFs), a fusion linker of five inert amino acids (gly-gly-ser-gly-gly; SEQ ID NO:29) was designed to link together the three ORFs within the fusion constructs. It is known that T-cell epitopes, peptides of 8-11 amino acids in length, prefer bulky or charged amino acids as anchors, commonly at peptide position 2 and at the COOH-terminus, to fit into MHC grooves. It is also know that the amino acid residues interacting with T-cell receptors, located between the two anchors, are usually charged amino acids. Thus, by introducing a stretch of five inert amino acids as a linker between two ORFs, the likelihood of a novel T-cell epitope with proper anchors and charged residues to interact with T-cell receptors is greatly reduced.

TABLE 7
Schematic representation of the HCMV antigen fusion constructs

| Fusion construct | Fusion scheme^{a} |
|---|---|
| P12 | M-mpp65-Linker-mIE1-Linker-mIE2 |
| P21 | M-mpp65-Linker-mIE2-Linker-mIE1 |
| 2P1 | M-mIE2-Linker-mpp65-Linker-mIE1 |
| 21P | M-mIE2-Linker-mIE1-Linker-mpp65 |

^{a}“Linker” signifies the amino acid sequence GGSGG (SEQ ID NO: 29).
“M” signifies a Methionine amino acid.

The amino acid sequence of a fusion protein encoded by the P12 fusion construct, designated herein as “mpp65-mIE1-mIE2,” is set forth as SEQ ID NO:20:

(SEQ ID NO: 20)

| | |
|---|---|
| 1 | MESRGRRCPE MISVLGPISG HVLKAVFSRG DTPVLPHETR |
| | LLQTGIHVRV |
| 51 | SQPSLILVSQ YTPDSTPCHR GDNQLQVQHT YFTGSEVENV |
| | SVNVHNPTGR |
| 101 | SICPSQEPMS IYVYALPLKM LNIPSINVHH YPSAAERKHR |
| | HLPVADAVIH |
| 151 | ASGKQMWQAR LTVSGLAWTR QQNQWKEPDV YYTSAFVFPT |
| | KDVALRHVVC |
| 201 | AHELVCSMEN TRATKMQVIG DQYVKVYLES FCEDVPSGKL |
| | FMHVTLGSDV |
| 251 | EEDLTMTRNP QPFMRPHERN GFTVLCPKNM IIKPGKISHI |
| | MLDVAFTSHE |
| 301 | HFGLLCPKSI PGLSISGNLL MNGQQIFLEV QAIRETVELR |
| | QYDPVAALFF |
| 351 | FDIDLLLQRG PQYSEHPTFT SQYRIQGKLE YRHTWDRHDE |
| | GAAQGDDDVW |
| 401 | TSGSDSDEEL VTTEGGTPGV TGGGAMAGAS TSAGRGRKSA |
| | SSATACTSGV |
| 451 | MTRGRLKAES TVAPEEDTDE DSDNEIHNPA VFTWPPWQAG |
| | ILARNLVPMV |
| 501 | ATVQGQNLKY QEFFWDANDI YRIFAELEGV WQPAAGGSGG |
| | PEKDVLAELV |
| 551 | KQIKVRVDMV RHRIKEHMLK KYTQTEEKFT GAFNMMGGCL |
| | QNALDILDKV |
| 601 | HEPFEEMKCI GLTMQSMYEN YIVPEDKREM WMACIKELHD |
| | VSKGAANKLG |
| 651 | GALQAKARAK KDELRRKMMY MCYRNIEFFT KNSAFPKTTN |
| | GCSQAMAALQ |
| 701 | NLPQCSPDEI MAYAQKIFKI LDEERDKVLT HIDHIFMDIL |
| | TTCVETMCNE |
| 751 | YKVTSDACMM TMYGGISLLS EFCRVLCCYV LEETSVMLAK |
| | RPLITKPEVI |
| 801 | SVMGGGIEEI SMKVFAQYIL GADPLRVCSP SVDDLRAIAE |
| | ESDEEEAIVA |
| 851 | YTLATAGVSS SDSLVSPPES PVPATIPLSS VIVAENSDQE |
| | ESEQSDEEEE |
| 901 | EGAQEEREDT VSVKSEPVSE IEEVAPEEEE DGAEEPTASG |
| | GKSTHPMVTR |
| 951 | SKADQGGSGG GDILAQAVNH AGIDSSSTGP TLTTHSCSVS |
| | SAPLNKPTPT |
| 1001 | SVAVTNTPLP GASATPELSP SSGPRKTTRP FKVIIKPPVP |
| | PAPIMLPLIK |
| 1051 | QEDIKPEPDF TIQYRNKIID TAGCIVISDS EEEQGEEVET |
| | RGATASSPST |
| 1101 | GSGTPRVTSP THPLSQMNHP PLPDPLGRPD EDSSSSSSSS |
| | CSSASDSESE |
| 1151 | SEEMKCSSGG GASVTSSHHG RGGFGGAASS SLLSCGHQSS |
| | GGASTGPRSS |
| 1201 | GSKRISELDN EKVRNIMKDK NTPFCTPNVQ TRRGRVKIDE |
| | VSRMFRNTNR |
| 1251 | SLEYKNLPFT IPSMHQVLDE AIKACKTMQV NNKGIQIIYT |
| | RNHEVKSEVD |
| 1301 | AVRCRLGTMC NLALSTPFLM EHTMPVTHPP EVAQRTADAC |
| | NEGVKAAWSL |
| 1351 | KELHTHQLCP RSSDYRNMII HAATPVDLLG ALNLCLPLMQ |
| | KFPKQVMVRI |
| 1401 | FSTNQGGFML PIYETAAKAY AVGQFEQPTE TPPEDLDTLS |
| | LAIEAAIQDL |
| 1451 | RNKSQ* |

The mpp65-mIE1-mIE2 protein is encoded by the nucleotide sequence as set forth in SEQ ID NO:21:

(SEQ ID NO: 21)
ATGGAGTCTCGTGGTCGTCGGTGCCCTGAGATGATCTCTGTGCTGGG
ACCCATCTCTGGCCATGTGCTGAAGGCTGTCTTCTCTCGGGGAGACA
CCCCTGTGCTGCCTCATGAGACCCGGCTGCTTCAGACAGGCATCCAT
GTGCGGGTCTCCCAGCCATCCCTGATCCTGGTCTCCCAGTACACCCC
TGACTCTACCCCATGCCATCGGGGTGACAACCAGCTTCAGGTGCAGC
ACACCTACTTCACAGGCTCTGAGGTGGAGAATGTCTCTGTGAATGTT
CACAACCCTACAGGCCGGTCCATCTGCCCATCCCAGGAGCCCATGTC
CATCTATGTCTATGCCCTGCCTCTGAAGATGCTGAACATCCCATCCA
TCAATGTGCATCACTACCCATCTGCTGCTGAGCGGAAGCATCGGCAT
CTGCCTGTGGCTGATGCTGTGATCCATGCCTCTGGCAAGCAGATGTG
GCAGGCTCGGCTGACAGTCTCTGGCCTGGCCTGGACTCGGCAGCAGA
ACCAGTGGAAGGAGCCTGATGTCTACTACACCTCTGCCTTTGTCTTC
CCCACCAAGGATGTGGCTCTGCGGCATGTGGTCTGTGCTCATGAGCT
GGTCTGCTCTATGGAGAACACTCGGGCCACCAAGATGCAGGTGATTG
GTGACCAGTATGTGAAGGTCTACCTGGAGTCCTTCTGTGAGGATGTG
CCATCTGGCAAGCTGTTCATGCATGTGACCCTGGGCTCTGATGTGGA
GGAGGACCTGACCATGACTCGGAACCCTCAGCCATTCATGCGGCCTC
ATGAGCGGAATGGCTTCACAGTGCTGTGCCCTAAGAACATGATCATC
AAGCCTGGCAAGATCAGCCACATCATGCTGGATGTGGCCTTCACCTC
CCATGAGCACTTTGGCCTGCTGTGCCCCAAGTCCATCCCTGGCCTGT
CCATCTCTGGCAACCTGCTGATGAATGGCCAGCAGATATTCCTGGAG
GTGCAGGCCATCCGGGAGACAGTGGAGCTGCGGCAGTATGACCCTGT
GGCTGCTCTGTTCTTCTTTGACATTGACCTGCTACTGCAGCGGGGCC
CTCAGTACTCTGAGCATCCCACCTTCACCTCCCAGTACCGTATCCAG
GGCAAGCTGGAGTACCGGCACACCTGGGACCGGCATGATGAGGGTGC
TGCCCAGGGTGATGATGATGTCTGGACCTCTGGCTCTGACTCTGATG
AGGAGCTGGTGACCACAGAGGGTGGCACCCCTGGTGTGACAGGTGGA
GGTGCTATGGCTGGTGCCTCCACCTCTGCTGGTCGGGGTCGGAAGTC
TGCCTCCTCTGCCACAGCTTGCACCTCTGGTGTGATGACTCGTGGTC
GGCTGAAGGCTGAGTCCACAGTGGCTCCTGAGGAGGACACAGATGAG
GACTCTGACAATGAGATCCACAACCCTGCTGTCTTCACCTGGCCTCC
ATGGCAGGCTGGCATCCTGGCTCGGAACCTGGTGCCTATGGTGGCCA
CAGTGCAGGGTCAGAACCTGAAGTACCAGGAGTTCTTCTGGGATGCC
AATGACATCTACCGGATCTTTGCTGAGCTGGAGGGTGTCTGGCAGCC
TGCTGCCGGTGGATCCGGTGGACCTGAGAAGGATGTGCTGGCTGAGC
TGGTGAAGCAGATCAAGGTGCGGGTGGACATGGTGCGGCATCGGATC
AAGGAGCACATGCTGAAGAAGTACACCCAGACAGAGGAGAAGTTCAC
AGGCGCCTTCAACATGATGGGTGGCTGCCTGCAGAATGCCCTGGACA
TCCTGGACAAGGTGCATGAGCCATTTGAGGAGATGAAGTGCATTGGC
CTGACCATGCAGTCCATGTATGAGAACTACATTGTGCCTGAGGACAA
GCGGGAGATGTGGATGGCCTGCATCAAGGAGCTGCATGATGTCTCCA
AGGGCGCTGCCAACAAGCTGGGCGGTGCCCTGCAGGCCAAGGCCCGG
GCCAAGAAGGATGAGCTGCGGCGGAAGATGATGTACATGTGCTACCG
GAACATTGAGTTCTTCACCAAGAACTCTGCCTTCCCCAAGACCACCA
ATGGCTGCTCCCAGGCCATGGCTGCCCTGCAGAACCTGCCCCAGTGC
TCCCCTGATGAGATCATGGCCTATGCCCAGAAGATATTCAAGATCCT
GGATGAGGAGCGGGACAAGGTGCTGACCCACATTGACCACATCTTCA
TGGACATCCTGACCACCTGTGTGGAGACCATGTGCAATGAGTACAAG
GTGACCTCTGATGCCTGCATGATGACCATGTATGGCGGCATCTCCCT
GCTGTCTGAGTTCTGCCGGGTGCTGTGCTGCTATGTGCTGGAGGAGA
CCTCTGTGATGCTGGCCAAGCGGCCCCTGATCACCAAGCCTGAGGTG
ATCTCTGTGATGGGTGGCGGTATTGAGGAGATCAGCATGAAGGTCTT
TGCCCAGTACATCCTGGGCGCTGACCCTCTGCGGGTCTGCTCCCCAT
CTGTGGATGACCTGCGGGCCATTGCTGAGGAGTCTGATGAGGAGGAG
GCCATTGTGGCCTACACCCTGGCCACAGCTGGCGTCTCCTCCTCTGA
CTCCCTGGTCTCCCCCCCTGAGTCCCCTGTGCCTGCCACCATCCCCC
TGTCCTCTGTGATTGTGGCTGAGAACTCTGACCAGGAGGAGTCTGAG
CAGTCTGATGAGGAGGAGGAGGAGGGTGCCCAGGAGGAGCGGGAGGA
CACAGTCTCTGTGAAGTCTGAGCCTGTCTCTGAGATTGAGGAGGTGG
CCCCTGAGGAGGAGGAGGATGGCGCTGAGGAGCCCACAGCCTCTGGC
GGCAAGTCCACCCATCCCATGGTGACCCGGTCCAAGGCTGACCAGGG
TGGTAGTGGAGGAGGCGACATCCTGGCCCAGGCTGTGAACCATGCTG
GCATTGACTCCTCCTCCACAGGCCCCACCCTGACCACCCACTCCTGC
TCTGTCTCCTCTGCCCCCCTGAACAAGCCCACCCCCACCTCTGTGGC
TGTGACCAACACCCCCCTGCCTGGCGCCTCTGCCACCCCTGAGCTGT
CCCCCTCTTCTGGTCCCCGGAAGACCACCCGGCCATTCAAGGTGATC
ATCAAGCCCCCTGTGCCCCCTGCCCCCATCATGCTGCCCCTGATCAA
GCAGGAGGACATCAAGCCTGAGCCTGACTTCACCATCCAGTACCGGA
ACAAGATCATTGACACAGCTGGCTGCATTGTGATCTCTGACTCTGAG
GAGGAGCAGGGCGAGGAGGTGGAGACCCGGGGCGCCACAGCCTCCTC
CCCATCCACAGGCTCTGGCACCCCCCGGGTGACCTCCCCCACCCATC
CCCTGTCCCAGATGAACCATCCCCCCCTGCCTGACCCCCTGGGCCGG
CCTGATGAGGACTCCTCCTCCTCCTCCTCCTCCTCCTGCTCCTCTGC
CTCTGACTCTGAGTCTGAGTCTGAGGAGATGAAGTGCTCCTCTGGCG
GCGGCGCCTCTGTGACCTCCTCCCATCATGGCCGGGGCGGCTTTGGC
GGCGCTGCCTCCTCCTCCCTGCTGTCCTGTGGCCATCAGTCCTCTGG
CGGCGCCTCCACAGGCCCCCGGTCTTCTGGTTCCAAGCGGATCTCTG
AGCTGGACAATGAGAAGGTGCGGAACATCATGAAGGACAAGAACACC
CCATTCTGCACCCCCAATGTGCAGACCCGGCGGGGCCGGGTGAAGAT
TGATGAGGTCTCCCGGATGTTCCGGAACACCAACCGGTCCCTGGAGT
ACAAGAACCTGCCATTCACCATCCCATCCATGCATCAGGTGCTGGAT
GAGGCCATCAAGGCCTGCAAGACCATGCAGGTGAACAACAAGGGCAT
CCAGATCATCTACACCCGGAACCATGAGGTGAAGTCTGAGGTGGATG
CTGTGCGGTGCCGGCTGGGCACCATGTGCAACCTGGCCCTGTCCACC
CCATTCCTGATGGAGCACACCATGCCTGTGACCCATCCCCCTGAGGT
GGCCCAGCGGACAGCTGATGCCTGCAATGAGGGCGTGAAGGCTGCCT
GGTCCCTGAAGGAGCTGCACACCCATCAGCTGTGCCCCCGGTCCTCT
GACTACCGGAACATGATCATCCATGCTGCCACCCCTGTGGACCTGCT
GGGCGCCCTGAACCTGTGCCTGCCCCTGATGCAGAAGTTCCCCAAGC
AGGTGATGGTGCGGATCTTCTCCACCAACCAGGGCGGCTTCATGCTG
CCCATCTATGAGACAGCTGCCAAGGCCTATGCTGTGGGCCAGTTTGA
GCAGCCCACAGAGACCCCCCCTGAGGACCTGGACACCCTGTCCCTGG
CCATTGAGGCTGCCATCCAGGACCTGCGGAACAAGTCCCAGTAA.

The amino acid sequence of a fusion protein encoded by the P21 fusion construct, designated herein as “mpp65-mIE2-mIE1,” is set forth as SEQ ID NO:22:

(SEQ ID NO: 22)

| | |
|---|---|
| 1 | MESRGRRCPE MISVLGPISG HVLKAVFSRG DTPVLPHETR |
| | LLQTGIHVRV |
| 51 | SQPSLILVSQ YTPDSTPCHR GDNQLQVQHT YFTGSEVENV |
| | SVNVHNPTGR |
| 101 | SICPSQEPMS IYVYALPLKM LNIPSINVHH YPSAAERKHR |
| | HLPVADAVIH |
| 151 | ASGKQMWQAR LTVSGLAWTR QQNQWKEPDV YYTSAFVFPT |
| | KDVALRHVVC |
| 201 | AHELVCSMEN TRATKMQVIG DQYVKVYLES FCEDVPSGKL |
| | FMHVTLGSDV |
| 251 | EEDLTMTRNP QPFMRPHERN GFTVLCPKNM IIKPGKISHI |
| | MLDVAFTSHE |
| 301 | HFGLLCPKSI PGLSISGNLL MNGQQIFLEV QAIRETVELR |
| | QYDPVAALFF |
| 351 | FDIDLLLQRG PQYSEHPTFT SQYRIQGKLE YRHTWDRHDE |
| | GAAQGDDDVW |
| 401 | TSGSDSDEEL VTTEGGTPGV TGGGAMAGAS TSAGRGRKSA |
| | SSATACTSGV |
| 451 | MTRGRLKAES TVAPEEDTDE DSDNEIHNPA VFTWPPWQAG |
| | ILARNLVPMV |
| 501 | ATVQGQNLKY QEFFWDANDI YRIFAELEGV WQPAAGGSGG |
| | GDILAQAVNH |
| 551 | AGIDSSSTGP TLTTHSCSVS SAPLNKPTPT SVAVTNTPLP |
| | GASATPELSP |
| 601 | SSGPRKTTRP FKVIIKPPVP PAPIMLPLIK QEDIKPEPDF |
| | TIQYRNKIID |
| 651 | TAGCIVISDS EEEQGEEVET RGATASSPST GSGTPRVTSP |
| | THPLSQMNHP |
| 701 | PLPDPLGRPD EDSSSSSSSS CSSASDSESE SEEMKCSSGG |
| | GASVTSSHHG |
| 751 | RGGFGGAASS SLLSCGHQSS GGASTGPRSS GSKRISELDN |
| | EKVRNIMKDK |
| 801 | NTPFCTPNVQ TRRGRVKIDE VSRMFRNTNR SLEYKNLPFT |
| | IPSMHQVLDE |
| 851 | AIKACKTMQV NNKGIQIIYT RNHEVKSEVD AVRCRLGTMC |
| | NLALSTPFLM |
| 901 | EHTMPVTHPP EVAQRTADAC NEGVKAAWSL KELHTHQLCP |
| | RSSDYRNMII |
| 951 | HAATPVDLLG ALNLCLPLMQ KFPKQVMVRI FSTNQGGFML |
| | PIYETAAKAY |
| 1001 | AVGQFEQPTE TPPEDLDTLS LAIEAAIQDL RNKSQGGSGG |
| | PEKDVLAELV |
| 1051 | KQIKVRVDMV RHRIKEHMLK KYTQTEEKFT GAFNMMGGCL |
| | QNALDILDKV |
| 1101 | HEPFEEMKCI GLTMQSMYEN YIVPEDKREM WMACIKELHD |
| | VSKGAANKLG |
| 1151 | GALQAKARAK KDELRRKMMY MCYRNIEFFT KNSAFPKTTN |
| | GCSQAMAALQ |
| 1201 | NLPQCSPDEI MAYAQKIFKI LDEERDKVLT HIDHIFMDIL |
| | TTCVETMCNE |
| 1251 | YKVTSDACMM TMYGGISLLS EFCRVLCCYV LEETSVMLAK |
| | RPLITKPEVI |
| 1301 | SVMGGGIEEI SMKVFAQYIL GADPLRVCSP SVDDLRAIAE |
| | ESDEEEAIVA |
| 1351 | YTLATAGVSS SDSLVSPPES PVPATIPLSS VIVAENSDQE |
| | ESEQSDEEEE |
| 1401 | EGAQEEREDT VSVKSEPVSE IEEVAPEEEE DGAEEPTASG |
| | GKSTHPMVTR |
| 1451 | SKADQ* |

The mpp65-mIE2-mIE1 protein is encoded by the nucleotide sequence as set forth in SEQ ID NO:23:

(SEQ ID NO: 23)
ATGGAGTCTCGTGGTCGTCGGTGCCCTGAGATGATCTCTGTGCTGGG
ACCCATCTCTGGCCATGTGCTGAAGGCTGTCTTCTCTCGGGGAGACA
CCCCTGTGCTGCCTCATGAGACCCGGCTGCTTCAGACAGGCATCCAT
GTGCGGGTCTCCCAGCCATCCCTGATCCTGGTCTCCCAGTACACCCC
TGACTCTACCCCATGCCATCGGGGTGACAACCAGCTTCAGGTGCAGC
ACACCTACTTCACAGGCTCTGAGGTGGAGAATGTCTCTGTGAATGTT
CACAACCCTACAGGCCGGTCCATCTGCCCATCCCAGGAGCCCATGTC
CATCTATGTCTATGCCCTGCCTCTGAAGATGCTGAACATCCCATCCA
TCAATGTGCATCACTACCCATCTGCTGCTGAGCGGAAGCATCGGCAT
CTGCCTGTGGCTGATGCTGTGATCCATGCCTCTGGCAAGCAGATGTG
GCAGGCTCGGCTGACAGTCTCTGGCCTGGCCTGGACTCGGCAGCAGA
ACCAGTGGAAGGAGCCTGATGTCTACTACACCTCTGCCTTTGTCTTC
CCCACCAAGGATGTGGCTCTGCGGCATGTGGTCTGTGCTCATGAGCT
GGTCTGCTCTATGGAGAACACTCGGGCCACCAAGATGCAGGTGATTG
GTGACCAGTATGTGAAGGTCTACCTGGAGTCCTTCTGTGAGGATGTG
CCATCTGGCAAGCTGTTCATGCATGTGACCCTGGGCTCTGATGTGGA
GGAGGACCTGACCATGACTCGGAACCCTCAGCCATTCATGCGGCCTC
ATGAGCGGAATGGCTTCACAGTGCTGTGCCCTAAGAACATGATCATC
AAGCCTGGCAAGATCAGCCACATCATGCTGGATGTGGCCTTCACCTC
CCATGAGCACTTTGGCCTGCTGTGCCCCAAGTCCATCCCTGGCCTGT
CCATCTCTGGCAACCTGCTGATGAATGGCCAGCAGATATTCCTGGAG
GTGCAGGCCATCCGGGAGACAGTGGAGCTGCGGCAGTATGACCCTGT
GGCTGCTCTGTTCTTCTTTGACATTGACCTGCTACTGCAGCGGGGCC
CTCAGTACTCTGAGCATCCCACCTTCACCTCCCAGTACCGTATCCAG
GGCAAGCTGGAGTACCGGCACACCTGGGACCGGCATGATGAGGGTGC
TGCCCAGGGTGATGATGATGTCTGGACCTCTGGCTCTGACTCTGATG
AGGAGCTGGTGACCACAGAGGGTGGCACCCCTGGTGTGACAGGTGGA
GGTGCTATGGCTGGTGCCTCCACCTCTGCTGGTCGGGGTCGGAAGTC
TGCCTCCTCTGCCACAGCTTGCACCTCTGGTGTGATGACTCGTGGTC
GGCTGAAGGCTGAGTCCACAGTGGCTCCTGAGGAGGACACAGATGAG
GACTCTGACAATGAGATCCACAACCCTGCTGTCTTCACCTGGCCTCC
ATGGCAGGCTGGCATCCTGGCTCGGAACCTGGTGCCTATGGTGGCCA
CAGTGCAGGGTCAGAACCTGAAGTACCAGGAGTTCTTCTGGGATGCC
AATGACATCTACCGGATCTTTGCTGAGCTGGAGGGTGTCTGGCAGCC
TGCTGCCGGTGGATCCGGTGGAGGCGACATCCTGGCCCAGGCTGTGA
ACCATGCTGGCATTGACTCCTCCTCCACAGGCCCCACCCTGACCACC
CACTCCTGCTCTGTCTCCTCTGCCCCCCTGAACAAGCCCACCCCCAC
CTCTGTGGCTGTGACCAACACCCCCCTGCCTGGCGCCTCTGCCACCC
CTGAGCTGTCCCCCTCTTCTGGTCCCCGGAAGACCACCCGGCCATTC
AAGGTGATCATCAAGCCCCCTGTGCCCCCTGCCCCCATCATGCTGCC
CCTGATCAAGCAGGAGGACATCAAGCCTGAGCCTGACTTCACCATCC
AGTACCGGAACAAGATCATTGACACAGCTGGCTGCATTGTGATCTCT
GACTCTGAGGAGGAGCAGGGCGAGGAGGTGGAGACCCGGGGCGCCAC
AGCCTCCTCCCCATCCACAGGCTCTGGCACCCCCCGGGTGACCTCCC
CCACCCATCCCCTGTCCCAGATGAACCATCCCCCCCTGCCTGACCCC
CTGGGCCGGCCTGATGAGGACTCCTCCTCCTCCTCCTCCTCCTCCTG
CTCCTCTGCCTCTGACTCTGAGTCTGAGTCTGAGGAGATGAAGTGCT
CCTCTGGCGGCGGCGCCTCTGTGACCTCCTCCCATCATGGCCGGGGC
GGCTTTGGCGGCGCTGCCTCCTCCTCCCTGCTGTCCTGTGGCCATCA
GTCCTCTGGCGGCGCCTCCACAGGCCCCCGGTCTTCTGGTTCCAAGC
GGATCTCTGAGCTGGACAATGAGAAGGTGCGGAACATCATGAAGGAC
AAGAACACCCCATTCTGCACCCCCAATGTGCAGACCCGGCGGGGCCG
GGTGAAGATTGATGAGGTCTCCCGGATGTTCCGGAACACCAACCGGT
CCCTGGAGTACAAGAACCTGCCATTCACCATCCCATCCATGCATCAG
GTGCTGGATGAGGCCATCAAGGCCTGCAAGACCATGCAGGTGAACAA
CAAGGGCATCCAGATCATCTACACCCGGAACCATGAGGTGAAGTCTG
AGGTGGATGCTGTGCGGTGCCGGCTGGGCACCATGTGCAACCTGGCC
CTGTCCACCCCATTCCTGATGGAGCACACCATGCCTGTGACCCATCC
CCCTGAGGTGGCCCAGCGGACAGCTGATGCCTGCAATGAGGGCGTGA
AGGCTGCCTGGTCCCTGAAGGAGCTGCACACCCATCAGCTGTGCCCC
CGGTCCTCTGACTACCGGAACATGATCATCCATGCTGCCACCCCTGT
GGACCTGCTGGGCGCCCTGAACCTGTGCCTGCCCCTGATGCAGAAGT
TCCCCAAGCAGGTGATGGTGCGGATCTTCTCCACCAACCAGGGCGGC
TTCATGCTGCCCATCTATGAGACAGCTGCCAAGGCCTATGCTGTGGG
CCAGTTTGAGCAGCCCACAGAGACCCCCCCTGAGGACCTGGACACCC
TGTCCCTGGCCATTGAGGCTGCCATCCAGGACCTGCGGAACAAGTCC
CAGGGTGGTAGTGGAGGACCTGAGAAGGATGTGCTGGCTGAGCTGGT
GAAGCAGATCAAGGTGCGGGTGGACATGGTGCGGCATCGGATCAAGG
AGCACATGCTGAAGAAGTACACCCAGACAGAGGAGAAGTTCACAGGC
GCCTTCAACATGATGGGTGGCTGCCTGCAGAATGCCCTGGACATCCT
GGACAAGGTGCATGAGCCATTTGAGGAGATGAAGTGCATTGGCCTGA
CCATGCAGTCCATGTATGAGAACTACATTGTGCCTGAGGACAAGCGG
GAGATGTGGATGGCCTGCATCAAGGAGCTGCATGATGTCTCCAAGGG
CGCTGCCAACAAGCTGGGCGGTGCCCTGCAGGCCAAGGCCCGGGCCA
AGAAGGATGAGCTGCGGCGGAAGATGATGTACATGTGCTACCGGAAC
ATTGAGTTCTTCACCAAGAACTCTGCCTTCCCCAAGACCACCAATGG
CTGCTCCCAGGCCATGGCTGCCCTGCAGAACCTGCCCCAGTGCTCCC
CTGATGAGATCATGGCCTATGCCCAGAAGATATTCAAGATCCTGGAT
GAGGAGCGGGACAAGGTGCTGACCCACATTGACCACATCTTCATGGA
CATCCTGACCACCTGTGTGGAGACCATGTGCAATGAGTACAAGGTGA
CCTCTGATGCCTGCATGATGACCATGTATGGCGGCATCTCCCTGCTG
TCTGAGTTCTGCCGGGTGCTGTGCTGCTATGTGCTGGAGGAGACCTC
TGTGATGCTGGCCAAGCGGCCCCTGATCACCAAGCCTGAGGTGATCT
CTGTGATGGGTGGCGGTATTGAGGAGATCAGCATGAAGGTCTTTGCC
CAGTACATCCTGGGCGCTGACCCTCTGCGGGTCTGCTCCCCATCTGT
GGATGACCTGCGGGCCATTGCTGAGGAGTCTGATGAGGAGGAGGCCA
TTGTGGCCTACACCCTGGCCACAGCTGGCGTCTCCTCCTCTGACTCC
CTGGTCTCCCCCCCTGAGTCCCCTGTGCCTGCCACCATCCCCCTGTC
CTCTGTGATTGTGGCTGAGAACTCTGACCAGGAGGAGTCTGAGCAGT
CTGATGAGGAGGAGGAGGAGGGTGCCCAGGAGGAGCGGGAGGACACA
GTCTCTGTGAAGTCTGAGCCTGTCTCTGAGATTGAGGAGGTGGCCCC
TGAGGAGGAGGAGGATGGCGCTGAGGAGCCCACAGCCTCTGGCGGCA
AGTCCACCCATCCCATGGTGACCCGGTCCAAGGCTGACCAGTAA.

The amino acid sequence of a fusion protein encoded by the 2P1 fusion construct, designated herein as “mIE2-mpp65-mIE1,” is set forth as SEQ ID NO:24:

(SEQ ID NO: 24)

| | |
|---|---|
| 1 | MGDILAQAVN HAGIDSSSTG PTLTTHSCSV SSAPLNKPTP |
| | TSVAVTNTPL |
| 51 | PGASATPELS PSSGPRKTTR PFKVIIKPPV PPAPIMLPLI |
| | KQEDIKPEPD |
| 101 | FTIQYRNKII DTAGCIVISD SEEEQGEEVE TRGATASSPS |
| | TGSGTPRVTS |
| 151 | PTHPLSQMNH PPLPDPLGRP DEDSSSSSSS SCSSASDSES |
| | ESEEMKCSSG |
| 201 | GGASVTSSHH GRGGFGGAAS SSLLSCGHQS SGGASTGPRS |
| | SGSKRISELD |
| 251 | NEKVRNIMKD KNTPFCTPNV QTRRGRVKID EVSRMFRNTN |
| | RSLEYKNLPF |
| 301 | TIPSMHQVLD EAIKACKTMQ VNNKGIQIIY TRNHEVKSEV |
| | DAVRCRLGTM |
| 351 | CNLALSTPFL MEHTMPVTHP PEVAQRTADA CNEGVKAAWS |
| | LKELHTHQLC |
| 401 | PRSSDYRNMI IHAATPVDLL GALNLCLPLM QKFPKQVMVR |
| | IFSTNQGGFM |
| 451 | LPIYETAAKA YAVGQFEQPT ETPPEDLDTL SLAIEAAIQD |
| | LRNKSQGGSG |
| 501 | GESRGRRCPE MISVLGPISG HVLKAVFSRG DTPVLPHETR |
| | LLQTGIHVRV |
| 551 | SQPSLILVSQ YTPDSTPCHR GDNQLQVQHT YFTGSEVENV |
| | SVNVHNPTGR |
| 601 | SICPSQEPMS IYVYALPLKM LNIPSINVHH YPSAAERKHR |
| | HLPVADAVIH |
| 651 | ASGKQMWQAR LTVSGLAWTR QQNQWKEPDV YYTSAFVFPT |
| | KDVALRHVVC |
| 701 | AHELVCSMEN TRATKMQVIG DQYVKVYLES FCEDVPSGKL |
| | FMHVTLGSDV |
| 751 | EEDLTMTRNP QPFMRPHERN GFTVLCPKNM IIKPGKISHI |
| | MLDVAFTSHE |
| 801 | HFGLLCPKSI PGLSISGNLL MNGQQIFLEV QAIRETVELR |
| | QYDPVAALFF |
| 851 | FDIDLLLQRG PQYSEHPTFT SQYRIQGKLE YRHTWDRHDE |
| | GAAQGDDDVW |
| 901 | TSGSDSDEEL VTTEGGTPGV TGGGAMAGAS TSAGRGRKSA |
| | SSATACTSGV |
| 951 | MTRGRLKAES TVAPEEDTDE DSDNEIHNPA VFTWPPWQAG |
| | ILARNLVPMV |
| 1001 | ATVQGQNLKY QEFFWDANDI YRIFAELEGV WQPAAGGSGG |
| | PEKDVLAELV |
| 1051 | KQIKVRVDMV RHRIKEHMLK KYTQTEEKFT GAFNMMGGCL |
| | QNALDILDKV |
| 1101 | HEPFEEMKCI GLTMQSMYEN YIVPEDKREM WMACIKELHD |
| | VSKGAANKLG |
| 1151 | GALQAKARAK KDELRRKMMY MCYRNIEFFT KNSAFPKTTN |
| | GCSQAMAALQ |
| 1201 | NLPQCSPDEI MAYAQKIFKI LDEERDKVLT HIDHIFMDIL |
| | TTCVETMCNE |
| 1251 | YKVTSDACMM TMYGGISLLS EFCRVLCCYV LEETSVMLAK |
| | RPLITKPEVI |
| 1301 | SVMGGGIEEI SMKVFAQYIL GADPLRVCSP SVDDLRAIAE |
| | ESDEEEAIVA |
| 1351 | YTLATAGVSS SDSLVSPPES PVPATIPLSS VIVAENSDQE |
| | ESEQSDEEEE |
| 1401 | EGAQEEREDT VSVKSEPVSE IEEVAPEEEE DGAEEPTASG |
| | GKSTHPMVTR |
| 1451 | SKADQ* |

The mIE2-mpp65-mIE1 protein is encoded by the nucleotide sequence as set forth in SEQ ID NO:25:

(SEQ ID NO: 25)
ATGGGCGACATCCTGGCCCAGGCTGTGAACCATGCTGGCATTGACTC
CTCCTCCACAGGCCCCACCCTGACCACCCACTCCTGCTCTGTCTCCT
CTGCCCCCCTGAACAAGCCCACCCCCACCTCTGTGGCTGTGACCAAC
ACCCCCCTGCCTGGCGCCTCTGCCACCCCTGAGCTGTCCCCCTCTTC
TGGTCCCCGGAAGACCACCCGGCCATTCAAGGTGATCATCAAGCCCC
CTGTGCCCCCTGCCCCCATCATGCTGCCCCTGATCAAGCAGGAGGAC
ATCAAGCCTGAGCCTGACTTCACCATCCAGTACCGGAACAAGATCAT
TGACACAGCTGGCTGCATTGTGATCTCTGACTCTGAGGAGGAGCAGG
GCGAGGAGGTGGAGACCCGGGGCGCCACAGCCTCCTCCCCATCCACA
GGCTCTGGCACCCCCCGGGTGACCTCCCCCACCCATCCCCTGTCCCA
GATGAACCATCCCCCCCTGCCTGACCCCCTGGGCCGGCCTGATGAGG
ACTCCTCCTCCTCCTCCTCCTCCTCCTGCTCCTCTGCCTCTGACTCT
GAGTCTGAGTCTGAGGAGATGAAGTGCTCCTCTGGCGGCGGCGCCTC
TGTGACCTCCTCCCATCATGGCCGGGGCGGCTTTGGCGGCGCTGCCT
CCTCCTCCCTGCTGTCCTGTGGCCATCAGTCCTCTGGCGGCGCCTCC
ACAGGCCCCCGGTCTTCTGGTTCCAAGCGGATCTCTGAGCTGGACAA
TGAGAAGGTGCGGAACATCATGAAGGACAAGAACACCCCATTCTGCA
CCCCCAATGTGCAGACCCGGCGGGGCCGGGTGAAGATTGATGAGGTC
TCCCGGATGTTCCGGAACACCAACCGGTCCCTGGAGTACAAGAACCT
GCCATTCACCATCCCATCCATGCATCAGGTGCTGGATGAGGCCATCA
AGGCCTGCAAGACCATGCAGGTGAACAACAAGGGCATCCAGATCATC
TACACCCGGAACCATGAGGTGAAGTCTGAGGTGGATGCTGTGCGGTG
CCGGCTGGGCACCATGTGCAACCTGGCCCTGTCCACCCCATTCCTGA
TGGAGCACACCATGCCTGTGACCCATCCCCCTGAGGTGGCCCAGCGG
ACAGCTGATGCCTGCAATGAGGGCGTGAAGGCTGCCTGGTCCCTGAA
GGAGCTGCACACCCATCAGCTGTGCCCCCGGTCCTCTGACTACCGGA
ACATGATCATCCATGCTGCCACCCCTGTGGACCTGCTGGGCGCCCTG
AACCTGTGCCTGCCCCTGATGCAGAAGTTCCCCAAGCAGGTGATGGT
GCGGATCTTCTCCACCAACCAGGGCGGCTTCATGCTGCCCATCTATG
AGACAGCTGCCAAGGCCTATGCTGTGGGCCAGTTTGAGCAGCCCACA
GAGACCCCCCCTGAGGACCTGGACACCCTGTCCCTGGCCATTGAGGC
TGCCATCCAGGACCTGCGGAACAAGTCCCAGGGTGGATCCGGTGGAG
AGTCTCGTGGTCGTCGGTGCCCTGAGATGATCTCTGTGCTGGGACCC
ATCTCTGGCCATGTGCTGAAGGCTGTCTTCTCTCGGGGAGACACCCC
TGTGCTGCCTCATGAGACCCGGCTGCTTCAGACAGGCATCCATGTGC
GGGTCTCCCAGCCATCCCTGATCCTGGTCTCCCAGTACACCCCTGAC
TCTACCCCATGCCATCGGGGTGACAACCAGCTTCAGGTGCAGCACAC
CTACTTCACAGGCTCTGAGGTGGAGAATGTCTCTGTGAATGTTCACA
ACCCTACAGGCCGGTCCATCTGCCCATCCCAGGAGCCCATGTCCATC
TATGTCTATGCCCTGCCTCTGAAGATGCTGAACATCCCATCCATCAA
TGTGCATCACTACCCATCTGCTGCTGAGCGGAAGCATCGGCATCTGC
CTGTGGCTGATGCTGTGATCCATGCCTCTGGCAAGCAGATGTGGCAG
GCTCGGCTGACAGTCTCTGGCCTGGCCTGGACTCGGCAGCAGAACCA
GTGGAAGGAGCCTGATGTCTACTACACCTCTGCCTTTGTCTTCCCCA
CCAAGGATGTGGCTCTGCGGCATGTGGTCTGTGCTCATGAGCTGGTC
TGCTCTATGGAGAACACTCGGGCCACCAAGATGCAGGTGATTGGTGA
CCAGTATGTGAAGGTCTACCTGGAGTCCTTCTGTGAGGATGTGCCAT
CTGGCAAGCTGTTCATGCATGTGACCCTGGGCTCTGATGTGGAGGAG
GACCTGACCATGACTCGGAACCCTCAGCCATTCATGCGGCCTCATGA
GCGGAATGGCTTCACAGTGCTGTGCCCTAAGAACATGATCATCAAGC
CTGGCAAGATCAGCCACATCATGCTGGATGTGGCCTTCACCTCCCAT
GAGCACTTTGGCCTGCTGTGCCCCAAGTCCATCCCTGGCCTGTCCAT
CTCTGGCAACCTGCTGATGAATGGCCAGCAGATATTCCTGGAGGTGC
AGGCCATCCGGGAGACAGTGGAGCTGCGGCAGTATGACCCTGTGGCT
GCTCTGTTCTTCTTTGACATTGACCTGCTACTGCAGCGGGGCCCTCA
GTACTCTGAGCATCCCACCTTCACCTCCCAGTACCGTATCCAGGGCA
AGCTGGAGTACCGGCACACCTGGGACCGGCATGATGAGGGTGCTGCC
CAGGGTGATGATGATGTCTGGACCTCTGGCTCTGACTCTGATGAGGA
GCTGGTGACCACAGAGGGTGGCACCCCTGGTGTGACAGGTGGAGGTG
CTATGGCTGGTGCCTCCACCTCTGCTGGTCGGGGTCGGAAGTCTGCC
TCCTCTGCCACAGCTTGCACCTCTGGTGTGATGACTCGTGGTCGGCT
GAAGGCTGAGTCCACAGTGGCTCCTGAGGAGGACACAGATGAGGACT
CTGACAATGAGATCCACAACCCTGCTGTCTTCACCTGGCCTCCATGG
CAGGCTGGCATCCTGGCTCGGAACCTGGTGCCTATGGTGGCCACAGT
GCAGGGTCAGAACCTGAAGTACCAGGAGTTCTTCTGGGATGCCAATG
ACATCTACCGGATCTTTGCTGAGCTGGAGGGTGTCTGGCAGCCTGCT
GCCGGTGGTAGTGGAGGACCTGAGAAGGATGTGCTGGCTGAGCTGGT
GAAGCAGATCAAGGTGCGGGTGGACATGGTGCGGCATCGGATCAAGG
AGCACATGCTGAAGAAGTACACCCAGACAGAGGAGAAGTTCACAGGC
GCCTTCAACATGATGGGTGGCTGCCTGCAGAATGCCCTGGACATCCT
GGACAAGGTGCATGAGCCATTTGAGGAGATGAAGTGCATTGGCCTGA
CCATGCAGTCCATGTATGAGAACTACATTGTGCCTGAGGACAAGCGG
GAGATGTGGATGGCCTGCATCAAGGAGCTGCATGATGTCTCCAAGGG
CGCTGCCAACAAGCTGGGCGGTGCCCTGCAGGCCAAGGCCCGGGCCA
AGAAGGATGAGCTGCGGCGGAAGATGATGTACATGTGCTACCGGAAC
ATTGAGTTCTTCACCAAGAACTCTGCCTTCCCCAAGACCACCAATGG
CTGCTCCCAGGCCATGGCTGCCCTGCAGAACCTGCCCCAGTGCTCCC
CTGATGAGATCATGGCCTATGCCCAGAAGATATTCAAGATCCTGGAT
GAGGAGCGGGACAAGGTGCTGACCCACATTGACCACATCTTCATGGA
CATCCTGACCACCTGTGTGGAGACCATGTGCAATGAGTACAAGGTGA
CCTCTGATGCCTGCATGATGACCATGTATGGCGGCATCTCCCTGCTG
TCTGAGTTCTGCCGGGTGCTGTGCTGCTATGTGCTGGAGGAGACCTC
TGTGATGCTGGCCAAGCGGCCCCTGATCACCAAGCCTGAGGTGATCT
CTGTGATGGGTGGCGGTATTGAGGAGATCAGCATGAAGGTCTTTGCC
CAGTACATCCTGGGCGCTGACCCTCTGCGGGTCTGCTCCCCATCTGT
GGATGACCTGCGGGCCATTGCTGAGGAGTCTGATGAGGAGGAGGCCA
TTGTGGCCTACACCCTGGCCACAGCTGGCGTCTCCTCCTCTGACTCC
CTGGTCTCCCCCCCTGAGTCCCCTGTGCCTGCCACCATCCCCCTGTC
CTCTGTGATTGTGGCTGAGAACTCTGACCAGGAGGAGTCTGAGCAGT
CTGATGAGGAGGAGGAGGAGGGTGCCCAGGAGGAGCGGGAGGACACA
GTCTCTGTGAAGTCTGAGCCTGTCTCTGAGATTGAGGAGGTGGCCCC
TGAGGAGGAGGAGGATGGCGCTGAGGAGCCCACAGCCTCTGGCGGCA
AGTCCACCCATCCCATGGTGACCCGGTCCAAGGCTGACCAGTAA.

The amino acid sequence of a fusion protein encoded by the 21P fusion construct, designated herein as “mIE2-mIE1-mpp65,” is set forth as SEQ ID NO:26:

(SEQ ID NO: 26)

| | |
|---|---|
| 1 | MGDILAQAVN HAGIDSSSTG PTLTTHSCSV SSAPLNKPTP |
| | TSVAVTNTPL |
| 51 | PGASATPELS PSSGPRKTTR PFKVIIKPPV PPAPIMLPLI |
| | KQEDIKPEPD |
| 101 | FTIQYRNKII DTAGCIVISD SEEEQGEEVE TRGATASSPS |
| | TGSGTPRVTS |
| 151 | PTHPLSQMNH PPLPDPLGRP DEDSSSSSSS SCSSASDSES |
| | ESEEMKCSSG |
| 201 | GGASVTSSHH GRGGFGGAAS SSLLSCGHQS SGGASTGPRS |
| | SGSKRISELD |
| 251 | NEKVRNIMKD KNTPFCTPNV QTRRGRVKID EVSRMFRNTN |
| | RSLEYKNLPF |
| 301 | TIPSMHQVLD EAIKACKTMQ VNNKGIQIIY TRNHEVKSEV |
| | DAVRCRLGTM |
| 351 | CNLALSTPFL MEHTMPVTHP PEVAQRTADA CNEGVKAAWS |
| | LKELHTHQLC |
| 401 | PRSSDYRNMI IHAATPVDLL GALNLCLPLM QKFPKQVMVR |
| | IFSTNQGGFM |
| 451 | LPIYETAAKA YAVGQFEQPT ETPPEDLDTL SLAIEAAIQD |
| | LRNKSQGGSG |
| 501 | GPEKDVLAEL VKQIKVRVDM VRHRIKEHML KKYTQTEEKF |
| | TGAFNMMGGC |
| 551 | LQNALDILDK VHEPFEEMKC IGLTMQSMYE NYIVPEDKRE |
| | MWMACIKELH |
| 601 | DVSKGAANKL GGALQAKARA KKDELRRKMM YMCYRNIEFF |
| | TKNSAFPKTT |
| 651 | NGCSQAMAAL QNLPQCSPDE IMAYAQKIFK ILDEERDKVL |
| | THIDHIFMDI |
| 701 | LTTCVETMCN EYKVTSDACM MTMYGGISLL SEFCRVLCCY |
| | VLEETSVMLA |
| 751 | KRPLITKPEV ISVMGGGIEE ISMKVFAQYI LGADPLRVCS |
| | PSVDDLRAIA |
| 801 | EESDEEEAIV AYTLATAGVS SSDSLVSPPE SPVPATIPLS |
| | SVIVAENSDQ |
| 851 | EESEQSDEEE EEGAQEERED TVSVKSEPVS EIEEVAPEEE |
| | EDGAEEPTAS |
| 901 | GGKSTHPMVT RSKADQGGSG GESRGRRCPE MISVLGPISG |
| | HVLKAVFSRG |
| 951 | DTPVLPHETR LLQTGIHVRV SQPSLILVSQ YTPDSTPCHR |
| | GDNQLQVQHT |
| 1001 | YFTGSEVENV SVNVHNPTGR SICPSQEPMS IYVYALPLKM |
| | LNIPSINVHH |
| 1051 | YPSAAERKHR HLPVADAVIH ASGKQMWQAR LTVSGLAWTR |
| | QQNQWKEPDV |
| 1101 | YYTSAFVFPT KDVALRHVVC AHELVCSMEN TRATKMQVIG |
| | DQYVKVYLES |
| 1151 | FCEDVPSGKL FMHVTLGSDV EEDLTMTRNP QPFMRPHERN |
| | GFTVLCPKNM |
| 1201 | IIKPGKISHI MLDVAFTSHE HFGLLCPKSI PGLSISGNLL |
| | MNGQQIFLEV |
| 1251 | QAIRETVELR QYDPVAALFF FDIDLLLQRG PQYSEHPTFT |
| | SQYRIQGKLE |
| 1301 | YRHTWDRHDE GAAQGDDDVW TSGSDSDEEL VTTEGGTPGV |
| | TGGGAMAGAS |
| 1351 | TSAGRGRKSA SSATACTSGV MTRGRLKAES TVAPEEDTDE |
| | DSDNEIHNPA |
| 1401 | VFTWPPWQAG ILARNLVPMV ATVQGQNLKY QEFFWDANDI |
| | YRIFAELEGV |
| 1451 | WQPAA* |

The mIE2-mIE1-mpp65 protein is encoded by the nucleotide sequence as set forth in SEQ ID NO:27:

(SEQ ID NO: 27)
ATGGGCGACATCCTGGCCCAGGCTGTGAACCATGCTGGCATTGACTC
CTCCTCCACAGGCCCCACCCTGACCACCCACTCCTGCTCTGTCTCCT
CTGCCCCCCTGAACAAGCCCACCCCCACCTCTGTGGCTGTGACCAAC
ACCCCCCTGCCTGGCGCCTCTGCCACCCCTGAGCTGTCCCCCTCTTC
TGGTCCCCGGAAGACCACCCGGCCATTCAAGGTGATCATCAAGCCCC
CTGTGCCCCCTGCCCCCATCATGCTGCCCCTGATCAAGCAGGAGGAC
ATCAAGCCTGAGCCTGACTTCACCATCCAGTACCGGAACAAGATCAT
TGACACAGCTGGCTGCATTGTGATCTCTGACTCTGAGGAGGAGCAGG
GCGAGGAGGTGGAGACCCGGGGCGCCACAGCCTCCTCCCCATCCACA
GGCTCTGGCACCCCCCGGGTGACCTCCCCCACCCATCCCCTGTCCCA
GATGAACCATCCCCCCCTGCCTGACCCCCTGGGCCGGCCTGATGAGG
ACTCCTCCTCCTCCTCCTCCTCCTCCTGCTCCTCTGCCTCTGACTCT
GAGTCTGAGTCTGAGGAGATGAAGTGCTCCTCTGGCGGCGGCGCCTC
TGTGACCTCCTCCCATCATGGCCGGGGCGGCTTTGGCGGCGCTGCCT
CCTCCTCCCTGCTGTCCTGTGGCCATCAGTCCTCTGGCGGCGCCTCC
ACAGGCCCCCGGTCTTCTGGTTCCAAGCGGATCTCTGAGCTGGACAA
TGAGAAGGTGCGGAACATCATGAAGGACAAGAACACCCCATTCTGCA
CCCCCAATGTGCAGACCCGGCGGGGCCGGGTGAAGATTGATGAGGTC
TCCCGGATGTTCCGGAACACCAACCGGTCCCTGGAGTACAAGAACCT
GCCATTCACCATCCCATCCATGCATCAGGTGCTGGATGAGGCCATCA
AGGCCTGCAAGACCATGCAGGTGAACAACAAGGGCATCCAGATCATC
TACACCCGGAACCATGAGGTGAAGTCTGAGGTGGATGCTGTGCGGTG
CCGGCTGGGCACCATGTGCAACCTGGCCCTGTCCACCCCATTCCTGA
TGGAGCACACCATGCCTGTGACCCATCCCCCTGAGGTGGCCCAGCGG
ACAGCTGATGCCTGCAATGAGGGCGTGAAGGCTGCCTGGTCCCTGAA
GGAGCTGCACACCCATCAGCTGTGCCCCCGGTCCTCTGACTACCGGA
ACATGATCATCCATGCTGCCACCCCTGTGGACCTGCTGGGCGCCCTG
AACCTGTGCCTGCCCCTGATGCAGAAGTTCCCCAAGCAGGTGATGGT
GCGGATCTTCTCCACCAACCAGGGCGGCTTCATGCTGCCCATCTATG
AGACAGCTGCCAAGGCCTATGCTGTGGGCCAGTTTGAGCAGCCCACA
GAGACCCCCCCTGAGGACCTGGACACCCTGTCCCTGGCCATTGAGGC
TGCCATCCAGGACCTGCGGAACAAGTCCCAGGGTGGATCCGGTGGAC
CTGAGAAGGATGTGCTGGCTGAGCTGGTGAAGCAGATCAAGGTGCGG
GTGGACATGGTGCGGCATCGGATCAAGGAGCACATGCTGAAGAAGTA
CACCCAGACAGAGGAGAAGTTCACAGGCGCCTTCAACATGATGGGTG
GCTGCCTGCAGAATGCCCTGGACATCCTGGACAAGGTGCATGAGCCA
TTTGAGGAGATGAAGTGCATTGGCCTGACCATGCAGTCCATGTATGA
GAACTACATTGTGCCTGAGGACAAGCGGGAGATGTGGATGGCCTGCA
TCAAGGAGCTGCATGATGTCTCCAAGGGCGCTGCCAACAAGCTGGGC
GGTGCCCTGCAGGCCAAGGCCCGGGCCAAGAAGGATGAGCTGCGGCG
GAAGATGATGTACATGTGCTACCGGAACATTGAGTTCTTCACCAAGA
ACTCTGCCTTCCCCAAGACCACCAATGGCTGCTCCCAGGCCATGGCT
GCCCTGCAGAACCTGCCCCAGTGCTCCCCTGATGAGATCATGGCCTA
TGCCCAGAAGATATTCAAGATCCTGGATGAGGAGCGGGACAAGGTGC
TGACCCACATTGACCACATCTTCATGGACATCCTGACCACCTGTGTG
GAGACCATGTGCAATGAGTACAAGGTGACCTCTGATGCCTGCATGAT
GACCATGTATGGCGGCATCTCCCTGCTGTCTGAGTTCTGCCGGGTGC
TGTGCTGCTATGTGCTGGAGGAGACCTCTGTGATGCTGGCCAAGCGG
CCCCTGATCACCAAGCCTGAGGTGATCTCTGTGATGGGTGGCGGTAT
TGAGGAGATCAGCATGAAGGTCTTTGCCCAGTACATCCTGGGCGCTG
ACCCTCTGCGGGTCTGCTCCCCATCTGTGGATGACCTGCGGGCCATT
GCTGAGGAGTCTGATGAGGAGGAGGCCATTGTGGCCTACACCCTGGC
CACAGCTGGCGTCTCCTCCTCTGACTCCCTGGTCTCCCCCCCTGAGT
CCCCTGTGCCTGCCACCATCCCCCTGTCCTCTGTGATTGTGGCTGAG
AACTCTGACCAGGAGGAGTCTGAGCAGTCTGATGAGGAGGAGGAGGA
GGGTGCCCAGGAGGAGCGGGAGGACACAGTCTCTGTGAAGTCTGAGC
CTGTCTCTGAGATTGAGGAGGTGGCCCCTGAGGAGGAGGAGGATGGC
GCTGAGGAGCCCACAGCCTCTGGCGGCAAGTCCACCCATCCCATGGT
GACCCGGTCCAAGGCTGACCAGGGTGGTAGTGGAGGAGAGTCTCGTG
GTCGTCGGTGCCCTGAGATGATCTCTGTGCTGGGACCCATCTCTGGC
CATGTGCTGAAGGCTGTCTTCTCTCGGGGAGACACCCCTGTGCTGCC
TCATGAGACCCGGCTGCTTCAGACAGGCATCCATGTGCGGGTCTCCC
AGCCATCCCTGATCCTGGTCTCCCAGTACACCCCTGACTCTACCCCA
TGCCATCGGGGTGACAACCAGCTTCAGGTGCAGCACACCTACTTCAC
AGGCTCTGAGGTGGAGAATGTCTCTGTGAATGTTCACAACCCTACAG
GCCGGTCCATCTGCCCATCCCAGGAGCCCATGTCCATCTATGTCTAT
GCCCTGCCTCTGAAGATGCTGAACATCCCATCCATCAATGTGCATCA
CTACCCATCTGCTGCTGAGCGGAAGCATCGGCATCTGCCTGTGGCTG
ATGCTGTGATCCATGCCTCTGGCAAGCAGATGTGGCAGGCTCGGCTG
ACAGTCTCTGGCCTGGCCTGGACTCGGCAGCAGAACCAGTGGAAGGA
GCCTGATGTCTACTACACCTCTGCCTTTGTCTTCCCCACCAAGGATG
TGGCTCTGCGGCATGTGGTCTGTGCTCATGAGCTGGTCTGCTCTATG
GAGAACACTCGGGCCACCAAGATGCAGGTGATTGGTGACCAGTATGT
GAAGGTCTACCTGGAGTCCTTCTGTGAGGATGTGCCATCTGGCAAGC
TGTTCATGCATGTGACCCTGGGCTCTGATGTGGAGGAGGACCTGACC
ATGACTCGGAACCCTCAGCCATTCATGCGGCCTCATGAGCGGAATGG
CTTCACAGTGCTGTGCCCTAAGAACATGATCATCAAGCCTGGCAAGA
TCAGCCACATCATGCTGGATGTGGCCTTCACCTCCCATGAGCACTTT
GGCCTGCTGTGCCCCAAGTCCATCCCTGGCCTGTCCATCTCTGGCAA
CCTGCTGATGAATGGCCAGCAGATATTCCTGGAGGTGCAGGCCATCC
GGGAGACAGTGGAGCTGCGGCAGTATGACCCTGTGGCTGCTCTGTTC
TTCTTTGACATTGACCTGCTACTGCAGCGGGGCCCTCAGTACTCTGA
GCATCCCACCTTCACCTCCCAGTACCGTATCCAGGGCAAGCTGGAGT
ACCGGCACACCTGGGACCGGCATGATGAGGGTGCTGCCCAGGGTGAT
GATGATGTCTGGACCTCTGGCTCTGACTCTGATGAGGAGCTGGTGAC
CACAGAGGGTGGCACCCCTGGTGTGACAGGTGGAGGTGCTATGGCTG
GTGCCTCCACCTCTGCTGGTCGGGGTCGGAAGTCTGCCTCCTCTGCC
ACAGCTTGCACCTCTGGTGTGATGACTCGTGGTCGGCTGAAGGCTGA
GTCCACAGTGGCTCCTGAGGAGGACACAGATGAGGACTCTGACAATG
AGATCCACAACCCTGCTGTCTTCACCTGGCCTCCATGGCAGGCTGGC
ATCCTGGCTCGGAACCTGGTGCCTATGGTGGCCACAGTGCAGGGTCA
GAACCTGAAGTACCAGGAGTTCTTCTGGGATGCCAATGACATCTACC
GGATCTTTGCTGAGCTGGAGGGTGTCTGGCAGCCTGCTGCCTAA.

Having described different embodiments of the invention, it is to be understood that the invention is not limited to those precise embodiments, and that various changes and modifications may be effected therein by one skilled in the art without departing from the scope or spirit of the invention as defined in the appended claims.

1. A nucleic acid molecule comprising a sequence of nucleotides that encodes a variant human cytomegalovirus (HCMV) protein selected from the group consisting of:
(a) a variant pp65 protein, wherein said variant comprises mutations relative to a wild-type pp65 amino acid sequence that eliminate or reduce bipartite nuclear localization signal (NLS) activity of the encoded pp65 variant, and wherein the variant pp65 is capable of producing an immune response in a mammal;
(b) a variant IE1 protein, wherein said variant comprises mutations relative to a wild-type IE1 amino acid sequence that eliminate or reduce bipartite NLS activity, and wherein the variant IE1 protein is capable of producing an immune response in a mammal; and
(c) a variant IE2 protein, wherein said variant comprises mutations relative to a wild-type IE2 amino acid sequence that eliminate or reduce bipartite NLS activity, and wherein the variant IE2 protein is capable of producing an immune response in a mammal

2. The nucleic acid molecule of claim 1, wherein said sequence of nucleotides encodes an amino acid sequence selected from the group consisting of: SEQ ID NOs: 3, 9, 16, 20, 22, 24, 26, 5, 10, 17, 21, 23, 25, and 27.

3. (canceled)

4. The nucleic acid molecule of claim 1, wherein the sequence of nucleotides encodes a variant pp65 protein and the mutations that eliminate or reduce NLS activity comprise one or more amino acid substitutions or deletions within approximately amino acids 415-438 of wild-type pp65 and one or more amino acid substitutions or deletions within approximately amino acids 536-561 of wild-type pp65.

5. The nucleic acid molecule of claim 4, wherein the mutations that eliminate or reduce NLS activity comprise substitutions R415G, K416G, and R419G, and a deletion of amino acids 536-561 of wild-type pp65.

6. The nucleic acid molecule of claim 5, wherein the variant pp65 further comprises a mutation at amino acid 436 of wild-type pp65 that eliminates or reduces the protein's putative kinase activity.

7. The nucleic acid molecule of claim 6, wherein the mutation that eliminates or reduces the protein's putative kinase activity comprises substitution K436G.

8. The nucleic acid molecule of claim 4, wherein the variant pp65 protein comprises an amino acid sequence that is at least 95% identical to the amino acid sequence as set forth in SEQ ID NO:3.

9. The nucleic acid molecule of claim 1, wherein the sequence of nucleotides encodes variant IE1 protein and further comprises a mutation that eliminates or reduces exon 3 activity of the protein.

10. The nucleic acid molecule of claim 9, wherein the mutations comprise one or more amino acid substitutions or deletions within approximately amino acids 2-25 of wild-type IE1 and one or more amino acid substitutions or deletions within approximately amino acids 326-342 of wild-type E1.

11. (canceled)

12. The nucleic acid molecule of claim 9, wherein the variant IE1 protein comprises an amino acid sequence that is at least 95% identical to the amino acid sequence as set forth in SEQ ID NO:9.

13. The nucleic acid molecule of claim 1, wherein the sequence nucleotides encodes a variant IE2 protein and the mutations that eliminate or reduce NLS activity comprise one or more amino acid substitutions or deletions within approximately amino acids 145-155 of wild-type IE2 and one or more amino acid substitutions or deletions within approximately amino acids 322-329 of wild-type IE2.

14.-16. (canceled)

17. The nucleic acid molecule of claim 13, wherein the variant IE2 protein comprises an amino acid sequence that is at least 95% identical to the amino acid sequence as set forth in SEQ ID NO:16.

18. The nucleic acid molecule of claim 1, wherein said sequence of nucleotides encodes a fusion protein comprising at least two of said (a), said (b), or said (c) variant HCMV protein fused together.

19. (canceled)

20. The nucleic acid molecule of claim 18, wherein
(i) the variant pp65 protein mutations comprise substitutions R415G, K416G, R419G, and K436G, and a deletion of amino acids 536-561;
(ii) the variant IE1 protein mutations comprise substitutions K340G, R341G, and R342G, and a deletion of amino acids 2-76; and,
(iii) the variant IE2 protein mutations comprise substitutions R146S, K147S, K148G, K324S, K325S, and K326G, and a deletion of amino acids 2-85.

21. (canceled)

22. The nucleic acid molecule of claim 20, wherein the fusion protein comprises an amino acid sequence that is at least 95% identical to an amino acid sequence selected from the group consisting of: SEQ ID NO:20, SEQ ID NO:22, SEQ ID NO:24, and SEQ ID NO:26.

23. (canceled)

24. A purified protein encoded by any of the nucleic acid molecules of claim 1.

25. A vector comprising any of the nucleic acid molecules of claim 1.

26.-27. (canceled)

28. A process for expressing a variant HCMV pp65, IE1, or IE2 protein, or a fusion protein thereof, in a recombinant host cell, comprising:
(a) introducing a vector of claim 25 into a suitable host cell; and,
(b) culturing the host cell under conditions which allow expression of the encoded, variant HCMV protein or fusion protein.

29. A pharmaceutical composition comprising the vector of claim 25 and a pharmaceutically acceptable carrier.

30. A method of treating a patient comprising the step of administering to said patient an effective amount of the pharmaceutical composition of claim 29.


SEQUENCE LISTING (29 sequences)

SEQ ID NO: 1 - protein, 561 aa, cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 2 - DNA, 1686 bp, cytomegalovirus
atggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcttggtatcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaggagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcagatcttcctggaggtacaagccatacgcgagaccgtggaactgcgtcagtacgatcccgtggctgcgctcttctttttcgatatcgacttgctgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgcaagacgccccgcgtcaccggcggcggcgccatggcgggcgcctccacttccgcgggccgcaaacgcaaatcagcatcctcggcgacggcgtgcacgtcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggaattcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttga

SEQ ID NO: 3 - protein, 535 aa, Artificial Sequence
  mpp65
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTEGGTPGVTGGGAMAGASTSAGRGRKSASSATACTSGVMTRGRLKAESTVAPEEDTDEDSDNEIHNPAVFTWPPWQAGILARNLVPMVATVQGQNLKYQEFFWDANDIYRIFAELEGVWQPAA

SEQ ID NO: 4 - DNA, 1605 bp, Artificial Sequence
  mpp65 (nuc)
atggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcttggtatcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaggagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcagatcttcctggaggtacaagccatacgcgagaccgtggaactgcgtcagtacgatcccgtggctgcgctcttctttttcgatatcgacttgctgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagggcgggacgcccggcgtcaccggcggcggcgccatggcgggcgcctccacttccgcgggccgcggacgcaaatcagcatcctcggcgacggcgtgcacgtcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggaattcttctgggacgccaacgacatctaccgcatcttcgccgaattggaaggcgtatggcagcccgctgcg

SEQ ID NO: 5 - DNA, 1605 bp, Artificial Sequence
  mpp65.syn (nuc)
atggagtctcgtggtcgtcggtgccctgagatgatctctgtgctgggacccatctctggccatgtgctgaaggctgtcttctctcggggagacacccctgtgctgcctcatgagacccggctgcttcagacaggcatccatgtgcgggtctcccagccatccctgatcctggtctcccagtacacccctgactctaccccatgccatcggggtgacaaccagcttcaggtgcagcacacctacttcacaggctctgaggtggagaatgtctctgtgaatgttcacaaccctacaggccggtccatctgcccatcccaggagcccatgtccatctatgtctatgccctgcctctgaagatgctgaacatcccatccatcaatgtgcatcactacccatctgctgctgagcggaagcatcggcatctgcctgtggctgatgctgtgatccatgcctctggcaagcagatgtggcaggctcggctgacagtctctggcctggcctggactcggcagcagaaccagtggaaggagcctgatgtctactacacctctgcctttgtcttccccaccaaggatgtggctctgcggcatgtggtctgtgctcatgagctggtctgctctatggagaacactcgggccaccaagatgcaggtgattggtgaccagtatgtgaaggtctacctggagtccttctgtgaggatgtgccatctggcaagctgttcatgcatgtgaccctgggctctgatgtggaggaggacctgaccatgactcggaaccctcagccattcatgcggcctcatgagcggaatggcttcacagtgctgtgccctaagaacatgatcatcaagcctggcaagatcagccacatcatgctggatgtggccttcacctcccatgagcactttggcctgctgtgccccaagtccatccctggcctgtccatctctggcaacctgctgatgaatggccagcagatattcctggaggtgcaggccatccgggagacagtggagctgcggcagtatgaccctgtggctgctctgttcttctttgacattgacctgctactgcagcggggccctcagtactctgagcatcccaccttcacctcccagtaccgtatccagggcaagctggagtaccggcacacctgggaccggcatgatgagggtgctgcccagggtgatgatgatgtctggacctctggctctgactctgatgaggagctggtgaccacagagggtggcacccctggtgtgacaggtggaggtgctatggctggtgcctccacctctgctggtcggggtcggaagtctgcctcctctgccacagcttgcacctctggtgtgatgactcgtggtcggctgaaggctgagtccacagtggctcctgaggaggacacagatgaggactctgacaatgagatccacaaccctgctgtcttcacctggcctccatgtcaggctggcatcctggctcggaacctggtgcctatggtggccacagtgcagggtcagaacctgaagtaccaggagttcttctgggatgccaatgacatctaccggatctttgctgagctggagggtgtctgtcagcctgctgcc

SEQ ID NO: 6 - protein, 491 aa, cytomegalovirus
MESSAKRKMDPDNPDEGPSSKVPRPETPVTKATTFLQTMLRKEVNSQLSLGDPLFPELAEESLKTFEQVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 7 - DNA, 1476 bp, cytomegalovirus
atggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaa

SEQ ID NO: 8 - DNA, 1473 bp, Artificial Sequence
  IE1.syn
atggagtcctctgccaagcggaagatggaccctgacaaccctgatgagggcccatcctccaaggtgcctcggcctgagacccctgtgaccaaggccaccaccttcctgcagaccatgctgcggaaggaggtgaactcccagctgtccctgggcgaccctctgttccctgagctggctgaggagtccctgaagacctttgagcaggtgacagaggactgcaatgagaaccctgagaaggatgtgctggctgagctggtgaagcagatcaaggtgcgggtggacatggtgcggcatcggatcaaggagcacatgctgaagaagtacacccagacagaggagaagttcacaggcgccttcaacatgatgggtggctgcctgcagaatgccctggacatcctggacaaggtgcatgagccatttgaggagatgaagtgcattggcctgaccatgcagtccatgtatgagaactacattgtgcctgaggacaagcgggagatgtggatggcctgcatcaaggagctgcatgatgtctccaagggcgctgccaacaagctgggcggtgccctgcaggccaaggcccgggccaagaaggatgagctgcggcggaagatgatgtacatgtgctaccggaacattgagttcttcaccaagaactctgccttccccaagaccaccaatggctgctcccaggccatggctgccctgcagaacctgccccagtgctcccctgatgagatcatggcctatgcccagaagatattcaagatcctggatgaggagcgggacaaggtgctgacccacattgaccacatcttcatggacatcctgaccacctgtgtggagaccatgtgcaatgagtacaaggtgacctctgatgcctgcatgatgaccatgtatggcggcatctccctgctgtctgagttctgccgggtgctgtgctgctatgtgctggaggagacctctgtgatgctggccaagcggcccctgatcaccaagcctgaggtgatctctgtgatgaagcggcggattgaggagatcagcatgaaggtctttgcccagtacatcctgggcgctgaccctctgcgggtctgctccccatctgtggatgacctgcgggccattgctgaggagtctgatgaggaggaggccattgtggcctacaccctggccacagctggcgtctcctcctctgactccctggtctccccccctgagtcccctgtgcctgccaccatccccctgtcctctgtgattgtggctgagaactctgaccaggaggagtctgagcagtctgatgaggaggaggaggagggtgcccaggaggagcgggaggacacagtctctgtgaagtctgagcctgtctctgagattgaggaggtggcccctgaggaggaggaggatggcgctgaggagcccacagcctctggcggcaagtccacccatcccatggtgacccggtccaaggctgaccag

SEQ ID NO: 9 - protein, 416 aa, Artificial Sequence
  mIE1
MPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMGGGIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 10 - DNA, 1248 bp, Artificial Sequence
  mIE1 (nuc)
atgcctgagaaggatgtgctggctgagctggtgaagcagatcaaggtgcgggtggacatggtgcggcatcggatcaaggagcacatgctgaagaagtacacccagacagaggagaagttcacaggcgccttcaacatgatgggtggctgcctgcagaatgccctggacatcctggacaaggtgcatgagccatttgaggagatgaagtgcattggcctgaccatgcagtccatgtatgagaactacattgtgcctgaggacaagcgggagatgtggatggcctgcatcaaggagctgcatgatgtctccaagggcgctgccaacaagctgggcggtgccctgcaggccaaggcccgggccaagaaggatgagctgcggcggaagatgatgtacatgtgctaccggaacattgagttcttcaccaagaactctgccttccccaagaccaccaatggctgctcccaggccatggctgccctgcagaacctgccccagtgctcccctgatgagatcatggcctatgcccagaagatattcaagatcctggatgaggagcgggacaaggtgctgacccacattgaccacatcttcatggacatcctgaccacctgtgtggagaccatgtgcaatgagtacaaggtgacctctgatgcctgcatgatgaccatgtatggcggcatctccctgctgtctgagttctgccgggtgctgtgctgctatgtgctggaggagacctctgtgatgctggccaagcggcccctgatcaccaagcctgaggtgatctctgtgatgggtggcggtattgaggagatcagcatgaaggtctttgcccagtacatcctgggcgctgaccctctgcgggtctgctccccatctgtggatgacctgcgggccattgctgaggagtctgatgaggaggaggccattgtggcctacaccctggccacagctggcgtctcctcctctgactccctggtctccccccctgagtcccctgtgcctgccaccatccccctgtcctctgtgattgtggctgagaactctgaccaggaggagtctgagcagtctgatgaggaggaggaggagggtgcccaggaggagcgggaggacacagtctctgtgaagtctgagcctgtctctgagattgaggaggtggcccctgaggaggaggaggatggcgctgaggagcccacagcctctggcggcaagtccacccatcccatggtgacccggtccaaggctgaccag

SEQ ID NO: 11 - protein, 580 aa, cytomegalovirus
MESSAKRKMDPDNPDEGPSSKVPRPETPVTKATTFLQTMLRKEVNSQLSLGDPLFPELAEESLKTFEQVTEDCNENPEKDVLAELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 12 - DNA, 1743 bp, cytomegalovirus
atggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaa

SEQ ID NO: 13 - DNA, 1740 bp, Artificial Sequence
  IE2.syn
atggagtcctctgccaagcggaagatggaccctgacaaccctgatgagggcccatcctccaaggtgccccggcctgagacccctgtgaccaaggccaccaccttcctgcagaccatgctgcggaaggaggtgaactcccagctgtccctgggcgaccccctgttccctgagctggctgaggagtccctgaagacctttgagcaggtgacagaggactgcaatgagaaccctgagaaggatgtgctggctgagctgggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccccggaagaagccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggaagaagaagtccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggagcacaccatgcctgtgacccatccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccag

SEQ ID NO: 14 - protein, 580 aa, Artificial Sequence
  IE2(H2A)
MESSAKRKMDPDNPDEGPSSKVPRPETPVTKATTFLQTMLRKEVNSQLSLGDPLFPELAEESLKTFEQVTEDCNENPEKDVLAELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 15 - DNA, 1740 bp, Artificial Sequence
  IE2(H2A)(nuc)
atggagtcctctgccaagcggaagatggaccctgacaaccctgatgagggcccatcctccaaggtgccccggcctgagacccctgtgaccaaggccaccaccttcctgcagaccatgctgcggaaggaggtgaactcccagctgtccctgggcgaccccctgttccctgagctggctgaggagtccctgaagacctttgagcaggtgacagaggactgcaatgagaaccctgagaaggatgtgctggctgagctgggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccccggaagaagccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggaagaagaagtccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggaggccaccatgcctgtgacagccccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccag

SEQ ID NO: 16 - protein, 496 aa, Artificial Sequence
  mIE2
MGDILAQAVNHAGIDSSSTGPTLTTHSCSVSSAPLNKPTPTSVAVTNTPLPGASATPELSPSSGPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRSSGSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 17 - DNA, 1488 bp, Artificial Sequence
  mIE2 (nuc)
atgggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccctcttctggtccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggtcttctggttccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggagcacaccatgcctgtgacccatccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccag

SEQ ID NO: 18 - protein, 496 aa, Artificial Sequence
  mIE2 (H2A)
MGDILAQAVNHAGIDSSSTGPTLTTHSCSVSSAPLNKPTPTSVAVTNTPLPGASATPELSPSSGPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRSSGSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 19 - DNA, 1488 bp, Artificial Sequence
  mIE2 (H2A) (nuc)
atgggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccctcttctggtccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggtcttctggttccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggaggccaccatgcctgtgacagccccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccag

SEQ ID NO: 20 - protein, 1455 aa, Artificial Sequence
  p12 fusion protein
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTEGGTPGVTGGGAMAGASTSAGRGRKSASSATACTSGVMTRGRLKAESTVAPEEDTDEDSDNEIHNPAVFTWPPCQAGILARNLVPMVATVQGQNLKYQEFFWDANDIYRIFAELEGVCQPAAGGSGGPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMGGGIEEISMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQGGSGGGDILAQAVNHAGIDSSSTGPTLTTHSCSVSSAPLNKPTPTSVAVTNTPLPGASATPELSPSSGPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRSSGSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 21 - DNA, 4368 bp, Artificial Sequence
  p12 nuc
atggagtctcgtggtcgtcggtgccctgagatgatctctgtgctgggacccatctctggccatgtgctgaaggctgtcttctctcggggagacacccctgtgctgcctcatgagacccggctgcttcagacaggcatccatgtgcgggtctcccagccatccctgatcctggtctcccagtacacccctgactctaccccatgccatcggggtgacaaccagcttcaggtgcagcacacctacttcacaggctctgaggtggagaatgtctctgtgaatgttcacaaccctacaggccggtccatctgcccatcccaggagcccatgtccatctatgtctatgccctgcctctgaagatgctgaacatcccatccatcaatgtgcatcactacccatctgctgctgagcggaagcatcggcatctgcctgtggctgatgctgtgatccatgcctctggcaagcagatgtggcaggctcggctgacagtctctggcctggcctggactcggcagcagaaccagtggaaggagcctgatgtctactacacctctgcctttgtcttccccaccaaggatgtggctctgcggcatgtggtctgtgctcatgagctggtctgctctatggagaacactcgggccaccaagatgcaggtgattggtgaccagtatgtgaaggtctacctggagtccttctgtgaggatgtgccatctggcaagctgttcatgcatgtgaccctgggctctgatgtggaggaggacctgaccatgactcggaaccctcagccattcatgcggcctcatgagcggaatggcttcacagtgctgtgccctaagaacatgatcatcaagcctggcaagatcagccacatcatgctggatgtggccttcacctcccatgagcactttggcctgctgtgccccaagtccatccctggcctgtccatctctggcaacctgctgatgaatggccagcagatattcctggaggtgcaggccatccgggagacagtggagctgcggcagtatgaccctgtggctgctctgttcttctttgacattgacctgctactgcagcggggccctcagtactctgagcatcccaccttcacctcccagtaccgtatccagggcaagctggagtaccggcacacctgggaccggcatgatgagggtgctgcccagggtgatgatgatgtctggacctctggctctgactctgatgaggagctggtgaccacagagggtggcacccctggtgtgacaggtggaggtgctatggctggtgcctccacctctgctggtcggggtcggaagtctgcctcctctgccacagcttgcacctctggtgtgatgactcgtggtcggctgaaggctgagtccacagtggctcctgaggaggacacagatgaggactctgacaatgagatccacaaccctgctgtcttcacctggcctccatgtcaggctggcatcctggctcggaacctggtgcctatggtggccacagtgcagggtcagaacctgaagtaccaggagttcttctgggatgccaatgacatctaccggatctttgctgagctggagggtgtctgtcagcctgctgccggtggatccggtggacctgagaaggatgtgctggctgagctggtgaagcagatcaaggtgcgggtggacatggtgcggcatcggatcaaggagcacatgctgaagaagtacacccagacagaggagaagttcacaggcgccttcaacatgatgggtggctgcctgcagaatgccctggacatcctggacaaggtgcatgagccatttgaggagatgaagtgcattggcctgaccatgcagtccatgtatgagaactacattgtgcctgaggacaagcgggagatgtggatggcctgcatcaaggagctgcatgatgtctccaagggcgctgccaacaagctgggcggtgccctgcaggccaaggcccgggccaagaaggatgagctgcggcggaagatgatgtacatgtgctaccggaacattgagttcttcaccaagaactctgccttccccaagaccaccaatggctgctcccaggccatggctgccctgcagaacctgccccagtgctcccctgatgagatcatggcctatgcccagaagatattcaagatcctggatgaggagcgggacaaggtgctgacccacattgaccacatcttcatggacatcctgaccacctgtgtggagaccatgtgcaatgagtacaaggtgacctctgatgcctgcatgatgaccatgtatggcggcatctccctgctgtctgagttctgccgggtgctgtgctgctatgtgctggaggagacctctgtgatgctggccaagcggcccctgatcaccaagcctgaggtgatctctgtgatgggtggcggtattgaggagatcagcatgaaggtctttgcccagtacatcctgggcgctgaccctctgcgggtctgctccccatctgtggatgacctgcgggccattgctgaggagtctgatgaggaggaggccattgtggcctacaccctggccacagctggcgtctcctcctctgactccctggtctccccccctgagtcccctgtgcctgccaccatccccctgtcctctgtgattgtggctgagaactctgaccaggaggagtctgagcagtctgatgaggaggaggaggagggtgcccaggaggagcgggaggacacagtctctgtgaagtctgagcctgtctctgagattgaggaggtggcccctgaggaggaggaggatggcgctgaggagcccacagcctctggcggcaagtccacccatcccatggtgacccggtccaaggctgaccagggtggtagtggaggaggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccctcttctggtccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggtcttctggttccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggagcacaccatgcctgtgacccatccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccagtaa

SEQ ID NO: 22 - protein, 1455 aa, Artificial Sequence
  p21 fusion protein
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTEGGTPGVTGGGAMAGASTSAGRGRKSASSATACTSGVMTRGRLKAESTVAPEEDTDEDSDNEIHNPAVFTWPPCQAGILARNLVPMVATVQGQNLKYQEFFWDANDIYRIFAELEGVCQPAAGGSGGGDILAQAVNHAGIDSSSTGPTLTTHSCSVSSAPLNKPTPTSVAVTNTPLPGASATPELSPSSGPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRSSGSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQGGSGGPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMGGGIEEISMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 23 - DNA, 4368 bp, Artificial Sequence
  P21 nuc
atggagtctcgtggtcgtcggtgccctgagatgatctctgtgctgggacccatctctggccatgtgctgaaggctgtcttctctcggggagacacccctgtgctgcctcatgagacccggctgcttcagacaggcatccatgtgcgggtctcccagccatccctgatcctggtctcccagtacacccctgactctaccccatgccatcggggtgacaaccagcttcaggtgcagcacacctacttcacaggctctgaggtggagaatgtctctgtgaatgttcacaaccctacaggccggtccatctgcccatcccaggagcccatgtccatctatgtctatgccctgcctctgaagatgctgaacatcccatccatcaatgtgcatcactacccatctgctgctgagcggaagcatcggcatctgcctgtggctgatgctgtgatccatgcctctggcaagcagatgtggcaggctcggctgacagtctctggcctggcctggactcggcagcagaaccagtggaaggagcctgatgtctactacacctctgcctttgtcttccccaccaaggatgtggctctgcggcatgtggtctgtgctcatgagctggtctgctctatggagaacactcgggccaccaagatgcaggtgattggtgaccagtatgtgaaggtctacctggagtccttctgtgaggatgtgccatctggcaagctgttcatgcatgtgaccctgggctctgatgtggaggaggacctgaccatgactcggaaccctcagccattcatgcggcctcatgagcggaatggcttcacagtgctgtgccctaagaacatgatcatcaagcctggcaagatcagccacatcatgctggatgtggccttcacctcccatgagcactttggcctgctgtgccccaagtccatccctggcctgtccatctctggcaacctgctgatgaatggccagcagatattcctggaggtgcaggccatccgggagacagtggagctgcggcagtatgaccctgtggctgctctgttcttctttgacattgacctgctactgcagcggggccctcagtactctgagcatcccaccttcacctcccagtaccgtatccagggcaagctggagtaccggcacacctgggaccggcatgatgagggtgctgcccagggtgatgatgatgtctggacctctggctctgactctgatgaggagctggtgaccacagagggtggcacccctggtgtgacaggtggaggtgctatggctggtgcctccacctctgctggtcggggtcggaagtctgcctcctctgccacagcttgcacctctggtgtgatgactcgtggtcggctgaaggctgagtccacagtggctcctgaggaggacacagatgaggactctgacaatgagatccacaaccctgctgtcttcacctggcctccatgtcaggctggcatcctggctcggaacctggtgcctatggtggccacagtgcagggtcagaacctgaagtaccaggagttcttctgggatgccaatgacatctaccggatctttgctgagctggagggtgtctgtcagcctgctgccggtggatccggtggaggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccctcttctggtccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggtcttctggttccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggagcacaccatgcctgtgacccatccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccagggtggtagtggaggacctgagaaggatgtgctggctgagctggtgaagcagatcaaggtgcgggtggacatggtgcggcatcggatcaaggagcacatgctgaagaagtacacccagacagaggagaagttcacaggcgccttcaacatgatgggtggctgcctgcagaatgccctggacatcctggacaaggtgcatgagccatttgaggagatgaagtgcattggcctgaccatgcagtccatgtatgagaactacattgtgcctgaggacaagcgggagatgtggatggcctgcatcaaggagctgcatgatgtctccaagggcgctgccaacaagctgggcggtgccctgcaggccaaggcccgggccaagaaggatgagctgcggcggaagatgatgtacatgtgctaccggaacattgagttcttcaccaagaactctgccttccccaagaccaccaatggctgctcccaggccatggctgccctgcagaacctgccccagtgctcccctgatgagatcatggcctatgcccagaagatattcaagatcctggatgaggagcgggacaaggtgctgacccacattgaccacatcttcatggacatcctgaccacctgtgtggagaccatgtgcaatgagtacaaggtgacctctgatgcctgcatgatgaccatgtatggcggcatctccctgctgtctgagttctgccgggtgctgtgctgctatgtgctggaggagacctctgtgatgctggccaagcggcccctgatcaccaagcctgaggtgatctctgtgatgggtggcggtattgaggagatcagcatgaaggtctttgcccagtacatcctgggcgctgaccctctgcgggtctgctccccatctgtggatgacctgcgggccattgctgaggagtctgatgaggaggaggccattgtggcctacaccctggccacagctggcgtctcctcctctgactccctggtctccccccctgagtcccctgtgcctgccaccatccccctgtcctctgtgattgtggctgagaactctgaccaggaggagtctgagcagtctgatgaggaggaggaggagggtgcccaggaggagcgggaggacacagtctctgtgaagtctgagcctgtctctgagattgaggaggtggcccctgaggaggaggaggatggcgctgaggagcccacagcctctggcggcaagtccacccatcccatggtgacccggtccaaggctgaccagtaa

SEQ ID NO: 24 - protein, 1455 aa, Artificial Sequence
  2P1 fusion protein
MGDILAQAVNHAGIDSSSTGPTLTTHSCSVSSAPLNKPTPTSVAVTNTPLPGASATPELSPSSGPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRSSGSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQGGSGGESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTEGGTPGVTGGGAMAGASTSAGRGRKSASSATACTSGVMTRGRLKAESTVAPEEDTDEDSDNEIHNPAVFTWPPCQAGILARNLVPMVATVQGQNLKYQEFFWDANDIYRIFAELEGVCQPAAGGSGGPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMGGGIEEISMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 25 - DNA, 4368 bp, Artificial Sequence
  2P1 nuc
atgggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccctcttctggtccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggtcttctggttccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggagcacaccatgcctgtgacccatccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccagggtggatccggtggagagtctcgtggtcgtcggtgccctgagatgatctctgtgctgggacccatctctggccatgtgctgaaggctgtcttctctcggggagacacccctgtgctgcctcatgagacccggctgcttcagacaggcatccatgtgcgggtctcccagccatccctgatcctggtctcccagtacacccctgactctaccccatgccatcggggtgacaaccagcttcaggtgcagcacacctacttcacaggctctgaggtggagaatgtctctgtgaatgttcacaaccctacaggccggtccatctgcccatcccaggagcccatgtccatctatgtctatgccctgcctctgaagatgctgaacatcccatccatcaatgtgcatcactacccatctgctgctgagcggaagcatcggcatctgcctgtggctgatgctgtgatccatgcctctggcaagcagatgtggcaggctcggctgacagtctctggcctggcctggactcggcagcagaaccagtggaaggagcctgatgtctactacacctctgcctttgtcttccccaccaaggatgtggctctgcggcatgtggtctgtgctcatgagctggtctgctctatggagaacactcgggccaccaagatgcaggtgattggtgaccagtatgtgaaggtctacctggagtccttctgtgaggatgtgccatctggcaagctgttcatgcatgtgaccctgggctctgatgtggaggaggacctgaccatgactcggaaccctcagccattcatgcggcctcatgagcggaatggcttcacagtgctgtgccctaagaacatgatcatcaagcctggcaagatcagccacatcatgctggatgtggccttcacctcccatgagcactttggcctgctgtgccccaagtccatccctggcctgtccatctctggcaacctgctgatgaatggccagcagatattcctggaggtgcaggccatccgggagacagtggagctgcggcagtatgaccctgtggctgctctgttcttctttgacattgacctgctactgcagcggggccctcagtactctgagcatcccaccttcacctcccagtaccgtatccagggcaagctggagtaccggcacacctgggaccggcatgatgagggtgctgcccagggtgatgatgatgtctggacctctggctctgactctgatgaggagctggtgaccacagagggtggcacccctggtgtgacaggtggaggtgctatggctggtgcctccacctctgctggtcggggtcggaagtctgcctcctctgccacagcttgcacctctggtgtgatgactcgtggtcggctgaaggctgagtccacagtggctcctgaggaggacacagatgaggactctgacaatgagatccacaaccctgctgtcttcacctggcctccatgtcaggctggcatcctggctcggaacctggtgcctatggtggccacagtgcagggtcagaacctgaagtaccaggagttcttctgggatgccaatgacatctaccggatctttgctgagctggagggtgtctgtcagcctgctgccggtggtagtggaggacctgagaaggatgtgctggctgagctggtgaagcagatcaaggtgcgggtggacatggtgcggcatcggatcaaggagcacatgctgaagaagtacacccagacagaggagaagttcacaggcgccttcaacatgatgggtggctgcctgcagaatgccctggacatcctggacaaggtgcatgagccatttgaggagatgaagtgcattggcctgaccatgcagtccatgtatgagaactacattgtgcctgaggacaagcgggagatgtggatggcctgcatcaaggagctgcatgatgtctccaagggcgctgccaacaagctgggcggtgccctgcaggccaaggcccgggccaagaaggatgagctgcggcggaagatgatgtacatgtgctaccggaacattgagttcttcaccaagaactctgccttccccaagaccaccaatggctgctcccaggccatggctgccctgcagaacctgccccagtgctcccctgatgagatcatggcctatgcccagaagatattcaagatcctggatgaggagcgggacaaggtgctgacccacattgaccacatcttcatggacatcctgaccacctgtgtggagaccatgtgcaatgagtacaaggtgacctctgatgcctgcatgatgaccatgtatggcggcatctccctgctgtctgagttctgccgggtgctgtgctgctatgtgctggaggagacctctgtgatgctggccaagcggcccctgatcaccaagcctgaggtgatctctgtgatgggtggcggtattgaggagatcagcatgaaggtctttgcccagtacatcctgggcgctgaccctctgcgggtctgctccccatctgtggatgacctgcgggccattgctgaggagtctgatgaggaggaggccattgtggcctacaccctggccacagctggcgtctcctcctctgactccctggtctccccccctgagtcccctgtgcctgccaccatccccctgtcctctgtgattgtggctgagaactctgaccaggaggagtctgagcagtctgatgaggaggaggaggagggtgcccaggaggagcgggaggacacagtctctgtgaagtctgagcctgtctctgagattgaggaggtggcccctgaggaggaggaggatggcgctgaggagcccacagcctctggcggcaagtccacccatcccatggtgacccggtccaaggctgaccagtaa

SEQ ID NO: 26 - protein, 1455 aa, Artificial Sequence
  21P fusion protein
MGDILAQAVNHAGIDSSSTGPTLTTHSCSVSSAPLNKPTPTSVAVTNTPLPGASATPELSPSSGPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRSSGSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQGGSGGPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMGGGIEEISMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQGGSGGESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTEGGTPGVTGGGAMAGASTSAGRGRKSASSATACTSGVMTRGRLKAESTVAPEEDTDEDSDNEIHNPAVFTWPPCQAGILARNLVPMVATVQGQNLKYQEFFWDANDIYRIFAELEGVCQPAA

SEQ ID NO: 27 - DNA, 4368 bp, Artificial Sequence
  21P nuc
atgggcgacatcctggcccaggctgtgaaccatgctggcattgactcctcctccacaggccccaccctgaccacccactcctgctctgtctcctctgcccccctgaacaagcccacccccacctctgtggctgtgaccaacacccccctgcctggcgcctctgccacccctgagctgtccccctcttctggtccccggaagaccacccggccattcaaggtgatcatcaagccccctgtgccccctgcccccatcatgctgcccctgatcaagcaggaggacatcaagcctgagcctgacttcaccatccagtaccggaacaagatcattgacacagctggctgcattgtgatctctgactctgaggaggagcagggcgaggaggtggagacccggggcgccacagcctcctccccatccacaggctctggcaccccccgggtgacctcccccacccatcccctgtcccagatgaaccatccccccctgcctgaccccctgggccggcctgatgaggactcctcctcctcctcctcctcctcctgctcctctgcctctgactctgagtctgagtctgaggagatgaagtgctcctctggcggcggcgcctctgtgacctcctcccatcatggccggggcggctttggcggcgctgcctcctcctccctgctgtcctgtggccatcagtcctctggcggcgcctccacaggcccccggtcttctggttccaagcggatctctgagctggacaatgagaaggtgcggaacatcatgaaggacaagaacaccccattctgcacccccaatgtgcagacccggcggggccgggtgaagattgatgaggtctcccggatgttccggaacaccaaccggtccctggagtacaagaacctgccattcaccatcccatccatgcatcaggtgctggatgaggccatcaaggcctgcaagaccatgcaggtgaacaacaagggcatccagatcatctacacccggaaccatgaggtgaagtctgaggtggatgctgtgcggtgccggctgggcaccatgtgcaacctggccctgtccaccccattcctgatggagcacaccatgcctgtgacccatccccctgaggtggcccagcggacagctgatgcctgcaatgagggcgtgaaggctgcctggtccctgaaggagctgcacacccatcagctgtgcccccggtcctctgactaccggaacatgatcatccatgctgccacccctgtggacctgctgggcgccctgaacctgtgcctgcccctgatgcagaagttccccaagcaggtgatggtgcggatcttctccaccaaccagggcggcttcatgctgcccatctatgagacagctgccaaggcctatgctgtgggccagtttgagcagcccacagagaccccccctgaggacctggacaccctgtccctggccattgaggctgccatccaggacctgcggaacaagtcccagggtggatccggtggacctgagaaggatgtgctggctgagctggtgaagcagatcaaggtgcgggtggacatggtgcggcatcggatcaaggagcacatgctgaagaagtacacccagacagaggagaagttcacaggcgccttcaacatgatgggtggctgcctgcagaatgccctggacatcctggacaaggtgcatgagccatttgaggagatgaagtgcattggcctgaccatgcagtccatgtatgagaactacattgtgcctgaggacaagcgggagatgtggatggcctgcatcaaggagctgcatgatgtctccaagggcgctgccaacaagctgggcggtgccctgcaggccaaggcccgggccaagaaggatgagctgcggcggaagatgatgtacatgtgctaccggaacattgagttcttcaccaagaactctgccttccccaagaccaccaatggctgctcccaggccatggctgccctgcagaacctgccccagtgctcccctgatgagatcatggcctatgcccagaagatattcaagatcctggatgaggagcgggacaaggtgctgacccacattgaccacatcttcatggacatcctgaccacctgtgtggagaccatgtgcaatgagtacaaggtgacctctgatgcctgcatgatgaccatgtatggcggcatctccctgctgtctgagttctgccgggtgctgtgctgctatgtgctggaggagacctctgtgatgctggccaagcggcccctgatcaccaagcctgaggtgatctctgtgatgggtggcggtattgaggagatcagcatgaaggtctttgcccagtacatcctgggcgctgaccctctgcgggtctgctccccatctgtggatgacctgcgggccattgctgaggagtctgatgaggaggaggccattgtggcctacaccctggccacagctggcgtctcctcctctgactccctggtctccccccctgagtcccctgtgcctgccaccatccccctgtcctctgtgattgtggctgagaactctgaccaggaggagtctgagcagtctgatgaggaggaggaggagggtgcccaggaggagcgggaggacacagtctctgtgaagtctgagcctgtctctgagattgaggaggtggcccctgaggaggaggaggatggcgctgaggagcccacagcctctggcggcaagtccacccatcccatggtgacccggtccaaggctgaccagggtggtagtggaggagagtctcgtggtcgtcggtgccctgagatgatctctgtgctgggacccatctctggccatgtgctgaaggctgtcttctctcggggagacacccctgtgctgcctcatgagacccggctgcttcagacaggcatccatgtgcgggtctcccagccatccctgatcctggtctcccagtacacccctgactctaccccatgccatcggggtgacaaccagcttcaggtgcagcacacctacttcacaggctctgaggtggagaatgtctctgtgaatgttcacaaccctacaggccggtccatctgcccatcccaggagcccatgtccatctatgtctatgccctgcctctgaagatgctgaacatcccatccatcaatgtgcatcactacccatctgctgctgagcggaagcatcggcatctgcctgtggctgatgctgtgatccatgcctctggcaagcagatgtggcaggctcggctgacagtctctggcctggcctggactcggcagcagaaccagtggaaggagcctgatgtctactacacctctgcctttgtcttccccaccaaggatgtggctctgcggcatgtggtctgtgctcatgagctggtctgctctatggagaacactcgggccaccaagatgcaggtgattggtgaccagtatgtgaaggtctacctggagtccttctgtgaggatgtgccatctggcaagctgttcatgcatgtgaccctgggctctgatgtggaggaggacctgaccatgactcggaaccctcagccattcatgcggcctcatgagcggaatggcttcacagtgctgtgccctaagaacatgatcatcaagcctggcaagatcagccacatcatgctggatgtggccttcacctcccatgagcactttggcctgctgtgccccaagtccatccctggcctgtccatctctggcaacctgctgatgaatggccagcagatattcctggaggtgcaggccatccgggagacagtggagctgcggcagtatgaccctgtggctgctctgttcttctttgacattgacctgctactgcagcggggccctcagtactctgagcatcccaccttcacctcccagtaccgtatccagggcaagctggagtaccggcacacctgggaccggcatgatgagggtgctgcccagggtgatgatgatgtctggacctctggctctgactctgatgaggagctggtgaccacagagggtggcacccctggtgtgacaggtggaggtgctatggctggtgcctccacctctgctggtcggggtcggaagtctgcctcctctgccacagcttgcacctctggtgtgatgactcgtggtcggctgaaggctgagtccacagtggctcctgaggaggacacagatgaggactctgacaatgagatccacaaccctgctgtcttcacctggcctccatgtcaggctggcatcctggctcggaacctggtgcctatggtggccacagtgcagggtcagaacctgaagtaccaggagttcttctgggatgccaatgacatctaccggatctttgctgagctggagggtgtctgtcagcctgctgcctaa

SEQ ID NO: 28 - DNA, 4867 bp, Artificial Sequence
  V1Jns
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgctgcggccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 29 - protein, 5 aa, Artificial Sequence
  linker
GGSGG